(19) United States
(12) Patent Application Publication
(10) Pub. No.: US 2024/0254500 A1
(43) Pub. Date: Aug. 1, 2024

(54) INCREASING THE ACCUMULATION OF EPA AND DHA IN RECOMBINANT CAMELINA

(71) Applicant: Rothamsted Research Limited, Harpenden (GB)

(72) Inventors: Johnathan Andrew Napier, Preston (GB); Lihua Han, Bedford (GB); Olga Vladimirovna Sayanova, St. Albans (GB); Richard Philip Haslam, Aylesbury (GB); Susana Silvestre, Torres Vedras (PT)

(21) Appl. No.: 18/267,536

(22) PCT Filed: Dec. 17, 2021
(86) PCT No.: PCT/EP2021/086663

§ 371 Date: Jun. 15, 2023

(30) Foreign Application Priority Data
Dec. 18, 2020 (LU) 102308

(51) Int. Cl. C12N 15/82; C12N 9/02; C12N 9/10; C12N 15/52

(52) U.S. Cl. CPC C12N 15/8247; C12N 9/0071; C12N 9/1029; C12N 15/52; C12Y 114/19003; C12Y 114/19006

(57) ABSTRACT

The present invention relates to improved recombinant plants or plant cells for producing omega-3 long chain polyunsaturated fatty acids such as eicospentaenoic acid (EPA) and docosahexaenoic acid (DHA). The invention further relates to the oil produced by said recombinant oilseed plant or cell.

### CROSS REFERENCE TO RELATED APPLICATIONS

This application is a U.S. National Stage Application filed under 35 U.S.C. § 371, based on International Patent Application No. PCT/EP2021/086663, filed on Dec. 17, 2021, which claims priority to LU Patent Application No. 102308, filed on Dec. 18, 2020. The entire contents of each of the above applications are incorporated herein by reference.

### FIELD OF THE INVENTION

The present invention relates to improved recombinant plants or plant seeds with increased levels of omega-3 long chain polyunsaturated fatty acids (LCPUFAs) such as eicospentaenoic acid (EPA) and docosahexaenoic acid (DHA) and total omega-3 LCPUFAs. The invention also relates to such plants or plant seeds further expressing astaxanthin. The invention also relates to the oil produced by said recombinant oilseed plant or cell.

### BACKGROUND OF THE INVENTION

Omega-3 (n-3) long chain polyunsaturated fatty acids (≥C20; LC-PUFAs), eicosapentaenoic acid (EPA; 20:5Δ^{5,8,11,14,17}) and docosahexaenoic acid (DHA; 22:6Δ^{4,7,10,13,16,19}), are widely recognised as being essential components of a healthy, balanced diet, which contribute to a reduced risk of cardiovascular disease, and proper brain and retinal development (West et al., 2019; Napier et al., 2020). Currently these n-3 LC-PUFAs come from wild capture fisheries. Whilst the oceanic fish stocks are at their maximum levels of sustainable production, the global human population continues to grow, so farmed fish fed on alternative more sustainable sources of fish meal and oils are being sought to meet human requirements and demand (Tocher et al., 2019).

One approach, which has successfully gone from theoretical concept to commercial prototyping, is the use of recombinant plants to accumulate these valuable fatty acids in their seed oil (Napier et al., 2018; 2019). In such a scenario, genetic modification (GM) is used to introduce the non-native biosynthetic pathway for omega-3 LC-PUFAs into the nuclear genome of a suitable oilseed host, enabling the plant to convert endogenous C18 fatty acids into the more desirable C20+LC-PUFAs such as eicosapentaenoic acid (EPA; 20:5Δ5,8,11,14,17) and docosahexaenoic acid (DHA; 22:6Δ4,7,10,13,16,19) (Napier et al., 2018). In most cases, this recombinant pathway is encoded by genes originating from marine microalgae (such organisms are the primary producers of omega-3 LC-PUFAs), with their expression in the plant restricted to the seed (Petrie and Singh, 2011). By this method, several groups have demonstrated the feasibility of making significant amounts of EPA and/or DHA in the seed oils of both model plant species such as Arabidopsis (Petrie et al., 2012; Ruiz-Lopez et al., 2013), but also (to varying levels) in oilseed crops such as Linseed, Camelina and Canola (Abbadi et al., 2004; Petrie et al., 2014; Ruiz-Lopez et al., 2014; Walsh et al., 2016).

Abbadi et al. (Plant Cell. 2004 October; 16(10):2734-48. Epub 2004 Sep. 17) described attempts to produce EPA in the seeds of recombinant linseed, using a three-gene construct containing a Δ6-desaturase (D6D) from Phaeodactylum tricornutum (AY082393), A6-elongase (D6E) from Physcomitrella patens (AF428243) and A5-desaturase (D5D) from Phaeodactylum tricornutum (AY082392). Linseed was chosen as a host species for the seed-specific expression of these genes on account of the very high levels of endogenous substrate (ALA) for prospective conversion to EPA. However, despite the presence of almost 50% ALA in the seeds of developing linseed, less than 1% EPA (0.8% of total fatty acids) was generated. In addition, very high levels of the undesired biosynthetic intermediate the omega-6 fatty acid γ-linolenic acid (GLA) were reported (16.8% of total fatty acids). This simultaneous accumulation of high levels of GLA and low synthesis of EPA was ascribed by Abbadi et al. (Plant Cell. 2004 October; 16(10):2734-48. Epub 2004 Sep. 17) to the phospholipid-dependent substrate-requirements of the D6D.

Similar results were also reported by Wu et al. (Nat Biotechnol, 2005, 23:1013-7) who described the seed-specific expression of a 3 gene construct (D6D from Pythium irregulare, CAJ30866; D6E from Physcomitrella patens; D5D from Thraustochytrium, AX467713) in Brassica juncea, yielding 0.8% EPA but 27.7% of the undesirable omega-6 GLA. More complex gene constructs were also reported by Wu et al. in which they attempted to boost the accumulation of EPA in recombinant B. juncea. A four gene construct comprising the same D6D, D6E, D5D activities and additionally the FAD2 Δ12-desaturase from Calendula officinalis (AF343065) resulted in a small increase in EPA to 1.2% but also a concomitant increase in GLA to 29.4%. A five gene construct, comprising D6D, D6E, D5D, FAD2 and a second A6-elongase D6E #2 from Thraustochytrium (AX214454) had equally marginal impact on the fatty acid composition of the seeds of recombinant B. juncea, yielding 1.4% EPA and 28.6% GLA. A six gene construct, comprising the same D6D, D6E, D5D, FAD2, D6E #2 and a w3-desaturase w3D from Phytophthora infestans (CS160901), yielded the best levels of EPA at 8.1%—however, the levels of GLA remained high at 27.1%. In a further iteration, Wu et al. (Nat Biotechnol, 2005, 23:1013-7) also attempted to engineer the accumulation of both EPA and DHA, through the seed-specific expression of nine genes (D6D, D6E, D5D, FAD2, D6E #2, w3D, and additionally a Δ5-elongase (D5E) from fish (Oncorhynchus mykiss; CS020097), a Δ4-desaturase (D4D) from Thraustochytrium (AF489589), and an acyltransferase also from the same organism). This yielded B. juncea seeds containing on average 8.1% EPA and 0.2% DHA. Again, GLA levels remained markedly higher (27.3%). Wu et al. reported a maximal level of EPA observed in recombinant B. juncea as 15% and a maximal DHA level of 1.5% (based on individual plants for their nine gene construct.

Similar experiments were carried out in the model oilseed species Arabidopsis thaliana: Robert et al. (Functional Plant Biol, 2005, 32: 473-479) reported the low level accumulation of EPA (3.2% of total fatty acids) in the seeds of Arabidopsis on the expression of two genes, a bifunctional D6D/D5D from zebrafish (Danio rerio, AF309556) and a D6E from the nematode Caenhorabditis elegans (Z68749). Interestingly, this construct also showed significantly reduced accumulation of GLA, a fact that Robert et al. attributed to the acyl-CoA-dependent substrate requirement of the D6D/D5D. Further transformation of this EPA-accumulating Arabidopsis line with genes for DHA synthesis (D4D and D5E from Pavlova salina, AY926605, AY926606) resulted in a mean level of 0.3% DHA, again with basal levels of the unwanted co-product GLA (0.3%).

Very similar results were reported by Hoffmann et al. (J Biol Chem, 2008, 283:22352-62) who postulated that the use of an “acyl-CoA-dependent” pathway in recombinant plants would decrease the build-up of biosynthetic intermediates such as GLA whilst simultaneously increase the accumulation of EPA. However, the seed-specific expression in Arabidopsis of acyl-CoA-dependent D6D and D5D activities from Mantoniella squamata (AM949597, AM949596) (in conjunction with the previously described D6E from P. patens) yielded barely detectable levels of EPA (<0.1% of total seed fatty acids and <0.05% GLA. Analogous data have been reported by Ruiz-Lopez et al. (Recombinant Res. 2012 (doi:10.1007/s11248-012-9596-0)) who expressed a number of different gene combinations in Arabidopsis. Notably, a six gene construct comprising a D6D from Pythium irregulare, (CAJ30866); D6E from Physcomitrella patens (AF428243); D5D from Thraustochytrium, (AX467713); a bifunctional D12/15 desaturase from Acanthamoeba castellanii, EF017656; w3D from Phytophthora infestans (CS160901) and a second D6E from Thalassiosira pseudonana, (AY591337) yielded 2.5% EPA of total seed fatty acids with the concomitant accumulation of 13.3% GLA. In contrast, a four gene construct that contained an acyl-CoA-dependent D6D from Ostreococcus tauri (AY746357), D6E from Thalassiosira pseudonana (AY591337), D5D from Thraustochytrium, (AX467713) and FAD2 from Phytophtora sojae (CS423998) generated low levels of both EPA (2% of total fatty acids) and GLA (1.0%).

More recently, Cheng et al. (Recombinant Res, 2010, 19:221-9) reported the accumulation of EPA in recombinant Brassica carinata. For example, the seed-specific expression of 3 genes (D6D from Pythium irregulare, CAJ30866; D6E from Thalassiosira pseudonana, AY591337; D5D from Thraustochytrium, AX467713) resulted in a mean level of 2.3% EPA, with high level co-accumulation of GLA (17.6%). A four gene construct (D6D, D6E, D5D and w3D from Claviceps purpurea, EF536898) resulted in 4.2% EPA and 11.8% GLA, whilst a five gene construct (D6D, D6E, D5D, w3D and an additional w3-desaturase from Pythium irregular, (FB753541)) yielded 9.7% EPA and 11.1% GLA. Such levels are very similar to that observed with five and six gene constructs in B. juncea (Wu et al. 2005, Nat Biotechnol, 2005, 23:1013-7). Cheng et al. introduced a different 5 gene construct (D6D from Pythium irregulare, CAJ30866; D6E from Thraustochytrium, HC476134; D5D from Thraustochytrium, AX467713; FAD2 from Calendula officinalis, AF343065 and w3D from Pythium irregulare, FB753541) into two different cultivars of B. carinata, differing in their accumulation of the C22 monounsaturated fatty acid erucic acid. Expression of this construct in conventional high erucic acid B. carinata resulted again in a mean accumulation of 9.3% EPA and 18.2% GLA. Expression in the zero-erucic acid genotype yielded an increase in EPA though this genotype also resulted in the co-accumulation of high levels of GLA (26.9%).

Very recently, two different recombinant canola lines accumulating omega-3 LC-PUFAs have been granted deregulated status in the USA (meaning that they are approved to be grown commercially), also representing the first examples of GM crops with nutritional enhancement traits (reviewed in Napier et al., 2018, 2019). In either case, the engineered canola produced either EPA or DHA separately but not together in the same seed. It is worth noting with regard to the invention described herein that the canola variety of rapeseed naturally has very low levels of euricic acid <2% whereas Camelina oil has a euricic acid content of 3-4% of total fatty acids.

However, given the significant value of generating plants with high levels of omega-3 LCPUFAs in the food industry, animal nutrition and in pharmaceuticals, there exists a need to increase even further levels of these commercially useful fatty acids in plants and in particular to increase the levels of EPA and DHA produced simultaneously in the same seed.

The present invention addresses this need.

Astaxanthin is a high-value keto-carotenoid that is renowned for its commercial application in a number of industries, including aquaculture, food, cosmetic, nutraceutical and pharmaceutical. In aquaculture in particular, astaxanthin is an essential aquacultural food additive necessary to give the pinkish-red colour to the flesh of salmons, trout, ornamental fish, shrimp, lobster and crayfish that is required for consumer acceptance. (Lim K C et al., 20218).

In addition to it's use as a feed additive for aquaculture where it is mainly used in combination with EPA and DHA omega-3 oils, astaxanthin because of its antioxidant properties is also useful in nutraceutical formulations for human consumption. As a dietary supplement, astaxanthin has been identified to have anti-inflammatory, anti-ageing, immune system boosting, anticancer, sun-proofing and antidiabetic activities resulting from its potent antioxidant properties. Current natural sources of astaxanthin are simple microorganisms, including algae, fungi, yeast and bacteria. Commercial astaxanthin is derived from chemical synthesis or natural resources, such as red yeast and freshwater microalgae (Lim K C et al., 2018). It is not surprising therefore that the global market in astaxanthin is estimated to be $647.1 million.

Given the high demand for astaxanthin, and in particular natural sources of astaxanthin, a land-based source has become of increasing interest. However, astaxanthin is rarely found in land plants.

There therefore exists a need to develop an alternative source of astaxanthin, and in particular develop and increase levels of astaxanthin that can be produced from natural sources. The present invention additionally addresses these needs.

Furthermore, in some cases, it is also useful to add antioxidants to enhance the stability of plant oils having levels of unsaturated fatty acids like the omega-3 oilseeds disclosed herein. It would therefore be desirable for cost and product stability reasons to simultaneously produce omega-3 oils and astaxanthin in the same oilseed. The present invention also addresses this need. Due to the chemical nature of these two seed products being miscible it would be expected that the astaxanthin will co-extract with the oil using either using traditional pressing or commercial scale solvent extraction. This oil would have natural enhanced oxidative stability due to the presence of the astaxanthin.

### SUMMARY OF THE INVENTION

The invention relates generally to recombinant plants that have increased production of LC-PUFAs, in particular, omega-3 LC-PUFAs such as DHA and total omega-3 LCPUFAs.

In one aspect of the invention, there is provided a recombinant plant, part thereof or plant cell, wherein the plant, part thereof or cell expresses a nucleic acid construct comprising at least one nucleic acid sequence encoding at least a Δ6-elongase, a Δ5-desaturase and a Δ6-desaturase, and wherein the plant has a decreased very long chain fatty acid (VLCFA) content compared to a wild-type or control plant. By “decreased VLCFA content” is meant that the plant comprises reduced expression or activity of a gene encoding an enzyme involved in the synthesis of VLCFAs.

Preferably, the construct further comprises a nucleic acid sequence encoding a Δ15-desaturase and/or a w3-desaturase. Preferably, the Δ15-desaturase is an FAD3 Δ15-desaturase. More preferably, the Δ15-desaturase is derived from Perilla frutescens and wherein the w3-desaturase is derived from Hyaloperonospora parasitica or Phytophora infestans. In a further embodiment, the construct further comprises at least one of a Δ12-desaturase, a Δ5-elongase and a Δ4desaturase. In one embodiment, the Δ12-desaturase is derived from Phytophora sojae, the Δ5-elongase is derived from Ostreococcus tauri and the Δ4desaturase is derived from Ostreococcus RCC809.

In a further embodiment, the Δ6-elongase is derived from Physcomitrella patens, the Δ5-desaturase is derived from Thraustochytrium, the Δ6-desaturase is derived from Ostreococcus tauri or Mantoniella squamata.

More preferably, the Δ6-elongase comprises a sequence as defined in SEQ ID NO: 3 or a functional variant thereof, Δ5-desaturase comprises a sequence as defined in SEQ ID NO: 5 or a functional variant thereof, Δ6-desaturase comprises a sequence as defined in SEQ ID NO: 1 or 21 or 23 or a functional variant thereof, w3-desaturase comprises a sequence as defined in SEQ ID NO: 11 or 13 or a functional variant thereof, Δ15-desaturase comprises a sequence as defined in SEQ ID NO: 19 or a functional variant thereof, Δ4-desaturase comprises a sequence as defined in SEQ ID NO:18 or a functional variant thereof, Δ12-desaturase comprises a sequence as defined in SEQ ID NO: 9 or a functional variant thereof, and Δ5-elongase comprises a sequence as defined in SEQ ID NO:7 or a functional variant thereof.

In a further embodiment, the plant, part thereof or cell comprises at least one mutation in a gene encoding an enzyme involved in the synthesis of VLCFAs. As such, the mutation leads to a reduction in the activity of the gene. More preferably, the plant comprises at least one mutation in a gene encoding fatty acid elongase 1 (FAE1). Mutations of the FAE1 gene are known to reduce the levels of euricic acid in Camelina oil.

More preferably the mutation is a homozygous loss-of-function mutation. In one embodiment, the mutation is introduced using CRISPR/Cas9 to target at least one gene encoding FAE1, preferably all genes encoding FAE1, FAE1-A, FAE1-B and FAE1-C. In an alternative embodiment, the plant, part thereof or plant cell expresses a RNA interference construct, wherein the construct reduces or abolished the expression of at least one gene encoding an enzyme involved in the synthesis of VLCFAs. In a preferred embodiment, the CRISPR or RNAi construct is stably incorporated into the plant genome.

In one embodiment, the nucleic acid sequences are operably linked to one or more regulatory sequences. More preferably, the nucleic acid sequences are each operably linked to a regulatory sequence, where the regulatory sequence is selected from the unknown seed protein seed-specific promoter, the Napin seed specific promoter, the 25 seed storage protein (Conlinin) promoter, the 11S seed storage protein (Glycinin) promoter, the sucrose-binding protein promoter and the Arcelin-5 seed storage protein promoter. In a further embodiment, the construct further comprises a nucleic acid sequence encoding resistance to at least one herbicide. Preferably, the nucleic acid construct is stably incorporated into the plant genome.

Preferably, the plant has increased production of omega-3 LC-PUFAs. In one embodiment, the plant has increased production of DHA, wherein preferably the DHA content is at least 10%, more preferably 15% or more and even more preferably between 10 and 20% (mole %) of the total fatty acid content of the plant. In another an alternative or additional embodiment, the plant has increased production of EPA, wherein preferably the EPA content is at least 9%, more preferably 10% (mole %) or more of the total fatty acid content of the plant. In a further additional or alternative embodiment, the plant has decreased levels of Gondoic acid (GA), wherein the GA content is 5% (mole %) or less of the total fatty acid content of the plant.

In one embodiment, the plant has an increased production of EPA and DHA, wherein the combined total of EPA and DHA is more than 20%, more preferably more than 25% and even more preferably between 20 and 35% (mole %) of the total fatty acid content of the plant.

In another embodiment, the amount of total omega-3 fatty acids is increased, wherein more preferably the total omega-3 fatty acids is at least 40%, more preferably at least 50% and even more preferably between 40 and 60% (mole %) of the total fatty acid content of the plant.

In another embodiment, the amount of total omega-6 fatty acids is decreased, wherein more preferably the total omega-6 fatty acids is less than 25% (mole %) of the total fatty acid content of the plant. In another embodiment, the ratio of total C20+n-3/C20+n-6 fatty acids and/or the ratio of omega-3/omega-6 LC-PUFAs is increased.

In another aspect of the invention, there is provided the use of the recombinant plant of the present invention to produce or increase production of omega-3 LC-PUFAs and/or to increase the ratio of omega-3 to omega-6 fatty PUFAs.

In a further aspect of the invention there is provided a method of producing the recombinant plant of the present invention, the method comprising introducing and expressing a nucleic acid construct comprising at least one nucleic acid sequence encoding at least a Δ6-elongase, a Δ5-desaturase and a Δ6-desaturase before, after or concurrently with reducing or abolishing the expression at least one FAE1 gene.

In another aspect of the invention, there is provided a method of producing the recombinant plant of the present invention, the method comprising introducing and expressing a nucleic acid construct comprising at least one nucleic acid sequence encoding at least a Δ6-elongase, a Δ5-desaturase and a Δ6-desaturase in a first plant, reducing or abolishing the expression of at least one FAE1 gene in a second plant and crossing the first and second plant, wherein the progeny express the nucleic acid construct and have reduced or abolished expression of FAE1.

In another aspect of the invention, there is provided a method of producing a recombinant plant, part thereof or plant cell with increased omega-3 LC-PUFAs content, the method comprising cultivating the recombinant plant, part thereof or cell of the present invention under conditions which allow the production of one or more omega-3 LC-PUFAs, and obtaining said omega-3 LC-PUFAs from the plant, part thereof or cell.

In a further aspect of the invention, there is provided a method for producing one or more omega-3 LC-PUFA, the method comprising growing a plant of the present invention under conditions wherein said desaturase and elongase enzymes are expressed.

In another aspect of the invention, there is provided a method for increasing the amount of triacylglycerol (TAG) species of 56 carbons and above, the method comprising growing a plant according to the invention under conditions wherein said desaturase and elongase enzymes are expressed. In one embodiment, the method comprises increasing the amount of 62:16, 64:14, 64:17 and 66:17 TAG species. In another embodiment, the method comprises increasing the amount of one or more of EPA, DHA and DPA in C58 or more TAGs.

In another aspect of the invention, there is provided a method for producing plant seed oil, comprising growing a plant, part thereof or cell of the present invention under conditions wherein said desaturase and elongase enzymes are expressed and a plant seed oil is produced in said plant, part thereof or cell.

In a further aspect there is provided a plant seed oil produced by the method of the present invention. In one embodiment, the plant seed oil wherein the plant seed oil comprises DHA and DHA constitutes at least 10%, more preferably 15% or more and even more preferably between 10 and 20% (mole %) of the total fatty acid content present in said oil. In a further embodiment, the plant seed oil comprises EPA and EPA constitutes at least 9%, more preferably 10% (mole %) or more of the total fatty acid content present in said oil. In another embodiment, the plant seed oil comprises GA and GA constitutes 5% (mole %) or less of the total fatty acid content present in said oil.

In a further embodiment, the combined total of EPA and DHA in the plant seed oil constitutes more than 20%, more preferably more than 25% and even more preferably between 20 and 35% (mole %) of the total fatty acid content present in said oil.

Preferably the plant part thereof is a seed. Accordingly, in a further aspect of the invention, there is provided a seed obtained or obtainable by the plant of the present invention. There is also provided progeny obtained or obtainable from the plant seed of the invention as well as plant seed obtained or obtainable from the progeny.

In another aspect of the invention there is provided a feedstuff, food, cosmetic, nutraceutical or pharmaceutical comprising the oil of the present invention.

Preferably, the plant is selected from the family Brassicaceae. More preferably, the plant is Camelina.

### DESCRIPTION OF THE FIGURES

The invention is further described in the following non-limiting figures.

FIG. 1 shows A. omega-3 long chain polyunsaturated fatty acids metabolic pathway and fatty acids composition in Camelina sativa. B shows a schematic of the DHA2015.1 construct. C. shows the levels of fatty acids in DHAfae1 seeds.

FIG. 2 shows a schematic representation of the flux of substrate fatty acids through different pathways and routes (omega-3 vs omega-6). The enzymatic conversion of fatty acids and the various routes for substrate flux are indicated. The different transgene-encoded enzyme activities are represented by the coloured arrows and indicated in the figure. The point of disruption in the fae1 mutants is indicated.

FIG. 3 shows the improved fatty acid composition of DHA1 construct in the fae1 mutant background. A) Table of total seed fatty acids from single seeds of DHA1xfae1 line, compared with WT and DHA1 seeds. Seeds displaying a high accumulation of EPA and DHA are shown in bold. B) Further analysis of the fatty acid composition of individual seeds from DHA1xfae1 line, providing details of the total levels of EPA+DHA, omega-3s and omega-3/omega-6 ratio, compared with seeds from WT and DHA1. C) Example fatty acid profile for Seed #18 (EPA+DHA=27%), note the significantly reduced levels of 20:1n-9 (marked with a star), confirming the inactivation of the FAE1 pathway. D) Example fatty acid profile for Seed #34 (EPA+DHA=30%), note the significantly reduced levels of 20:1 n-9 (marked with a star) confirming the inactivation of the FAE1 pathway. E) Example fatty acid profile for Seed #44 (EPA+DHA=27%) note the significantly reduced levels of 20:1 n-9 (marked with a star) confirming the inactivation of the FAE1 pathway. F) Example fatty acid profile for=Seed #63 (EPA+DHA=27%) note the significantly reduced levels of 20:1n-9 (marked with a star) confirming the inactivation of the FAE1 pathway.

FIG. 4 shows a schematic representation of the nucleic acid constructs of the invention.

FIG. 5 shows the seed fatty acid composition in WT and different DHA lines in a 2018 field trial.

FIG. 6 shows the seed fatty acid composition in WT and different EPA lines in a 2018 field trial.

FIG. 7 shows the omega-3 LC PUFA content in DHA and EPA lines of different generations.

FIG. 8 shows the oil quality parameters of different genetically modified lines.

FIG. 9 shows the T3 generation of grown seeds fames data for the DHA lines.

FIG. 10 shows the T3 generation of grown seeds fames data for the EPA lines.

FIG. 11 shows the results from a 2018 field trial for different DHA constructs DHA2, DHA3, DHA4 and DHA5 with DHA1 FAMEs.

FIG. 12 shows the results from a 2018 field trial comparing the EPA constructs EPA4, EPA8 and EPA2016.1 with EPA_B4.1FAMEs.

FIG. 13 shows triacylglycerol profile from mature Camelina sativa seeds. TAG molecular species were characterised using a ESI-MS/MS neutral loss survey scan with each of the TAG species represented by the total number of fatty acid atoms:desaturations. (A) fae1 vs. DHA2015.1 vs. faelxDHA2015. (B) WTs vs.DHA2015.1 vs. faelxDHA2015.1. (C) DHA2015.1 vs. faelxDHA2015. Data points presented as nmol %, Variable N.

FIG. 14 shows the presence of EPA (A) DHA (B) and DPA (C) in each TAG species.

FIG. 15 shows fatty acids composition in astaxanthin (ASX) constructs.

FIG. 16 A: carotenoid content of cold-pressed and solvent extracted oil from CASX and DHA1 camelina plants. B: depicts the ASX-AS2 construct.

FIG. 17 shows A: the results of a cross between plants with the fae1 mutant background and plants with expressing the EPA8 construct. B: depicts the EPA8 construct.

FIG. 18 shows A: concentration of astaxanthin in μg/g in seeds from a fae1×CASX-1 cross (which is generated by a previous cross between DHA2015.1 and an ASX line). B: is a GC-FID analysis of seeds from the F2 cross between fae1 and the CASX line.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of microbiology, tissue culture, molecular biology, chemistry, biochemistry and recombinant DNA technology, which are within the skill of the art. Such techniques are explained fully in the literature.

Polyunsaturated fatty acids can be classified into two major families (depending on the position (n) of the first double bond nearest the methyl end of the fatty acid carbon chain. Thus, the omega-6 fatty acids have the first unsaturated double bond six carbon atoms from the omega (methyl) end of the molecule and additionally may have a total of two or more double bonds, with each subsequent unsaturation occurring 3 additional carbon atoms toward the carboxyl end of the molecule. In contrast, the omega-3 fatty acids have the first unsaturated double bond three carbon atoms away from the omega end of the molecule and additionally may have a total of three or more double bonds, with each subsequent unsaturation occurring 3 additional carbon atoms toward the carboxyl end of the molecule. Table 1 summarizes the common names of various fatty acids, including omega-3 fatty acids, and the abbreviations that will be used throughout the specification.

TABLE 1

| Common Name | Abbreviation | Shorthand notation |
|---|---|---|
| oleic acid | OA | 18:1^{Δ9} |
| Linoleic acid | LA | 18:2^{Δ9,12} |
| γ-Linolenic | GLA | 18:3^{Δ6,9,12} |
| di-homo γ-linolenic acid | DGLA | 20:3^{Δ8,11,14} |
| Arachidonic acid | ARA | 20:4^{Δ5,8,11,14} |
| α-linolenic acid | ALA | 18:3^{Δ9,12,15} |
| stearidonic acid | SDA | 18:4^{Δ6,9,12,15} |
| eicosatetraenoic acid | ETA | 20:4^{Δ8.11.14.17} |
| eicosapentaenoic acid | EPA | 20:5^{Δ5,8,11,14,17} |
| docosapentaenoic acid | DPA | 22:5^{Δ7,10,13,16,19} |
| docosahexaenoic acid | DHA | 22:6^{Δ4,7,10,13,16,19} |

The fatty acids produced by the processes of the present invention can be isolated from the plant in the form of an oil, a lipid or a free fatty acid. One embodiment of the invention is therefore oils, lipids or fatty acids or fractions thereof which have been produced by the methods of the invention, especially preferably oil, lipid or a fatty acid composition comprising EPA, DPA and/or DHA and being derived from a recombinant plant.

The term “oil”, or “lipid” is used herein to mean a fatty acid mixture comprising unsaturated, preferably esterified, fatty acid(s). The oil or lipid is preferably high in omega-3 polyunsaturated or, advantageously, esterified fatty acid(s). In a particularly preferred embodiment, the oil or lipid has a high ALA, ETA, EPA, DPA and/or DHA content, preferably a high EPA, DPA and/or DHA content.

For the analysis, the fatty acid content of the seed can for example, be determined by gas chromatography after converting the fatty acids into the methyl esters by transesterification of lipids such as triacylglycerides and/or phospholipids.

The omega-3 polyunsaturated acids produced in the method of the present invention, for example EPA, DPA and DHA, may be in the form of fatty acid derivatives, for example sphingolipids, phosphoglycerides, lipids, glycolipids, phospholipids, monoacylglycerol, diacylglycerol, triacylglycerol or other fatty acid esters.

The omega-3 and other polyunsaturated fatty acids which are present can be liberated for example via treatment with alkali, for example aqueous KOH or NaOH, or acid hydrolysis, advantageously in the presence of an alcohol such as methanol or ethanol, or via enzymatic cleavage, and isolated via, for example, phase separation and subsequent acidification via, for example, H₂SO₄. The fatty acids can also be liberated directly without the above-described processing step.

If further purification is necessary, standard methods can be employed. Such methods may include extraction, treatment with urea, fractional crystallization, HPLC, fractional distillation, silica gel chromatography, high-speed centrifugation or distillation, or combinations of these techniques. Protection of reactive groups, such as the acid or alkenyl groups, may be done at any step through known techniques (e.g., alkylation, iodination, use of butylated hydroxytoluene (BHT)). Methods used include methylation of the fatty acids to produce methyl esters. Similarly, protecting groups may be removed at any step. Desirably, purification of fractions containing, for example, ALA, STA, ETA, EPA, DPA and DHA may be accomplished by treatment with urea and/or fractional distillation.

The present invention encompasses the use of the oil, lipid, the fatty acids and/or the fatty acid composition in feedstuffs, foodstuffs, cosmetics or pharmaceuticals. The oils, lipids, fatty acids or fatty acid mixtures according to the invention can be used in the manner with which the skilled worker is familiar for mixing with other oils, lipids, fatty acids or fatty acid mixtures of animal origin, such as, for example, fish oils. Thus, the invention also provides feedstuffs, foodstuffs, cosmetics or pharmacologicals which comprise the oils, lipids, fatty acids or fatty acid mixtures of the present invention.

The term “total fatty acids content” herein refers to the sum of all (cellular) fatty acids and esters that can be derivatized to fatty acid methyl esters by the base transesterification method in a given sample (as known in the art, for example as described in Sayanova et al., (1997) Proc Natl Acad Sci USA. 1997 Apr. 15; 94(8):4211-6; Sayanova et al., (2003) FEBS Lett. 2003 May 8; 542(1-3):100-4).

The term “omega-3 fatty acid” herein refers to a fatty acid wherein the first unsaturated double bond, counting from the omega (methyl) end of the molecule, is three carbon atoms away from the omega end of the molecule. The term “amount of total omega-3 fatty acids” may refer to a combined amount of alpha-linoleic acid (18:3n3), stearidonic acid (18:4n3), eicosatrienoic acid (20:3n3), eicosatetraenoic acid (20:4n3), eicosapentaenoic acid (20:5n3), docosapentaenoic acid (22:5n3) and docosahexaenoic acid (22:6n3).

The term “omega-6 fatty acid” herein refers to a fatty acid wherein the first unsaturated double bond, counting from the omega (methyl) end of the molecule, is six carbon atoms away from the omega end of the molecule. The term “amount of total omega-6 fatty acids” may refer to a combined amount of linoleic acid (18:2n6), gamma-linolenic acid (18:3n6), eicosadienoic acid (20:2n6), dihomo-gamma-linolenic acid (20:3n6) and arachidonic acid (20:4n6)

The term “very long chain fatty acid” herein refers to a fatty acid with 20 or more carbon atoms. As used herein, a “decreased very long chain fatty acid content” may refer to a decreased content of fatty acids with 20 or more carbon atoms, preferably a decreased content of saturated and monounsaturated fatty acids with 20 or more carbon atoms, and more preferably a decreased content of saturated and monounsaturated fatty acids with 20 or more carbon atoms wherein the monosaturated fatty acid is an omega-9 and/or omega-11 fatty acid.

The term Astaxanthin refers to a “carotenoid”. The term “carotenoid” herein refers to a class of tetraterpenoid compounds containing a 40-carbon chain core structure and a conjugated carbon double-bond system. Carotenoids may be oxygenated at various positions on the core structure (e.g. by carbonyl groups or hydroxyl groups). Carotenoid content may be quantified by separating a given sample using liquid chromatography (e.g. UHPLC), detecting carotenoid peaks using a photodiode array detector (e.g. scanning at wavelengths between 250 to 600 nm), and obtaining a carotenoid content by analysis of the carotenoid peaks (e.g. by analysis of the area under the carotenoid peaks). Such methods are known to the person skilled in the art (e.g. using methods from Nogueira et al., 2013, The Plant Cell, 25 (11), 4560-4579).

The term “ketocarotenoid” herein refers to a class of carotenoid compounds containing one or more carbonyl groups. Ketocarotenoid content may be quantified by separating a given sample using liquid chromatography (e.g. UHPLC), detecting ketocarotenoid peaks using a photodiode array detector (e.g. scanning at wavelengths between 250 to 600 nm), and obtaining a ketocarotenoid content by analysis of the ketocarotenoid peaks (e.g. by analysis of the area under the ketocarotenoid peaks). Such methods are known to the person skilled in the art (e.g. using methods from Nogueira et al., 2013, The Plant Cell, 25(11), 4560-4579).

The term “desaturase” refers to a polypeptide component of a multi-enzyme complex that can desaturate, i.e., introduce a double bond in one or more fatty acids to produce a mono- or polyunsaturated fatty acid or precursor of interest. Some desaturases have activity on two or more substrates. It may be desirable to empirically determine the specificity of a fatty acid desaturase by transforming a suitable host with the gene for the fatty acid desaturase and determining its effect on the fatty acid profile of the host. Desaturases include omega-3-desaturase, Δ6-desaturase, Δ5-desaturase, Δ12-desaturase, Δ15-desaturase and Δ4-desaturase.

The term “elongase” as used herein refers to a polypeptide that can elongate a fatty acid carbon chain to produce an acid two carbons longer than the fatty acid substrate that the elongase acts upon. Nucleic acids that encode for elongases isolated from various organisms can be used according to the various aspects of the invention and examples are described herein, including Ostreococcus sp. Examples of reactions catalyzed by elongase systems are the conversion of GLA to DGLA, SDA to ETA, ARA to DTA and EPA to DPA. In general, the substrate selectivity of elongases is somewhat broad but segregated by both chain length and the degree and type of unsaturation. Elongases include Δ6- and Δ5-elongases.

According to all aspects of the invention, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ. In a preferred embodiment, the regulatory sequence is a promoter. Preferably, the nucleic acid sequences are each operably linked to a regulatory sequence, where the regulatory sequence is selected from the unknown seed protein seed-specific promoter, the Napin seed specific promoter, the 25 seed storage protein (Conlinin) promoter, the 11S seed storage protein (Glycinin) promoter, the sucrose-binding protein promoter and the Arcelin-5 seed storage protein promoter.

The term “operably linked” as used herein refers to a functional linkage between the promoter sequence and the gene of interest, such that the promoter sequence is able to initiate transcription of the gene of interest.

The term “variant” or “functional variant” as used herein with reference to any of the sequences described herein refers to a variant gene sequence or part of the gene sequence which retains the biological function of the full non-variant sequence. A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence that result in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms.

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12 hours. Stringent conditions may also be achieved with the addition of destabilizing agents such as formamide. In one example, the stringent conditions may comprise hybridisation in 0.1×SPPE (or 0.1×SSC) and 0.1% SDS solution in a DNA or RNA hybridisation experiment at 65° C. and washing.

As used herein, by increase in levels of omega-3 LC-PUFAs is meant one or more omega-3 LC-PUFAs (such, as for example, one or more omega-3 LC-PUFA or total omega-3 LC-PUFA content). According to the various aspects of the invention, the omega-3 LC-PUFAs may be selected from SDA, ETA, EPA, DPA or DHA. In one embodiment, the omega-3 LC-PUFAs is DHA. In another embodiment, the omega-3 fatty acid is EPA. In another embodiment, omega-3 fatty acid is DHA. In one embodiment, the omega-3 fatty acid is DPA.

According to the various aspects of the invention described herein, the increase in the production of DHA, DPA or EPA is measured as an individual content of different omega-3 LC-PUFAs in total fatty acids (TFA), as described above. In other words, the increase is measured as a percentage of the total fatty acid content. Preferably, the increase is at least 2%, 3%, 4%, 5%, 6%, 7%, 8%, 9%, 10%, 11%, 12%, 13%, 14%, 15% or more compared to a control plant (mol %). In one embodiment, the control plant may express the DHA2015.1 or EPA_B4.1 construct. In another embodiment, the control plant may have one or more mutation in a fatty elongase 1 (FAE1 gene).

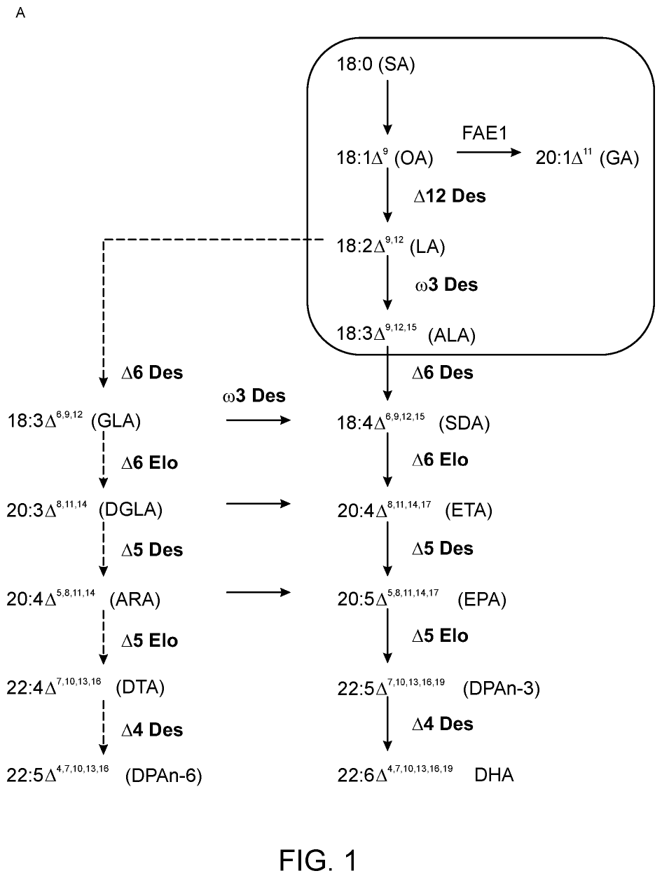

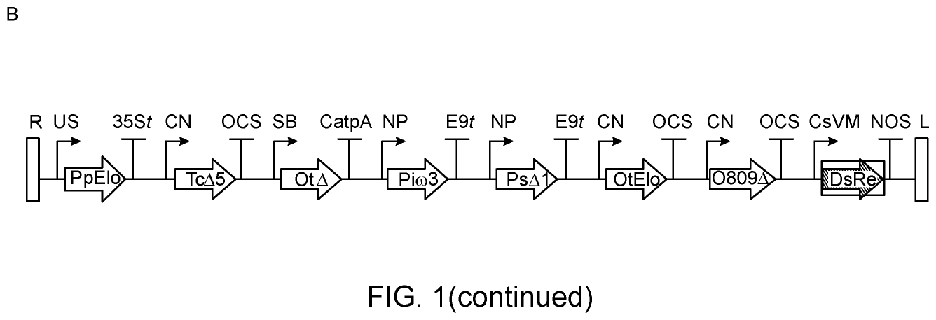

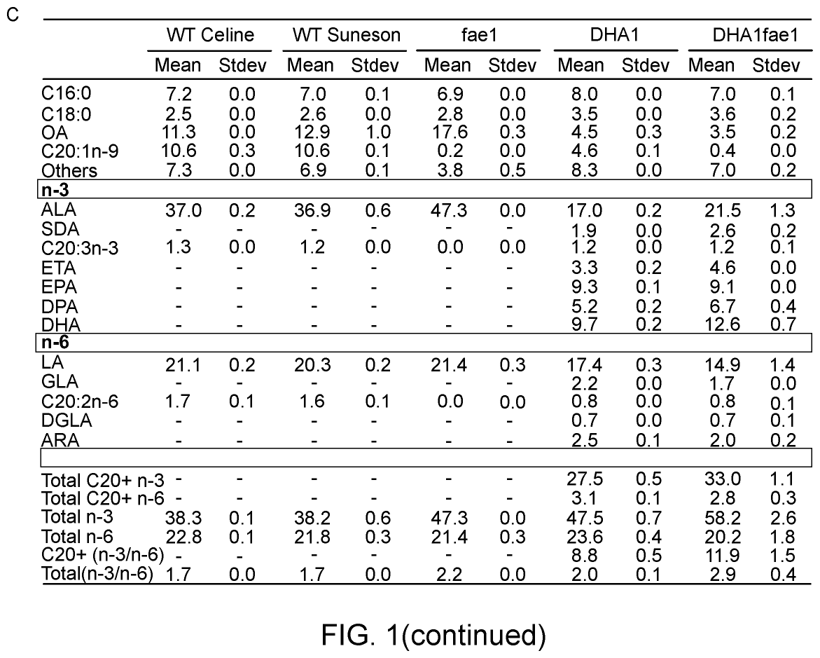

A control plant as used herein according to all of aspects of the invention is a plant, which has not been modified according to the methods of the invention. Accordingly, in one embodiment, the control plant does not express one of the constructs of the invention. In one embodiment, the control plant is a wild type plant. The control plant is typically of the same plant species, preferably having the same genetic background as the modified plant. Camelina sativa is an oilseed crop with naturally high levels of α-linolenic acid (ALA; 18:3Δ^{9,12,15}) with over 35% in seed oil. By a series of successive desaturation and elongation steps, ALA can be converted into EPA and DHA and these two fatty acids are considered to be the key constituents of fish oils (FIG. 1a) (Ruiz-Lopez et al., 2014; Petrie et al., 2014). We have successfully reconstituted the EPA and DHA biosynthetic pathway in Camelina cv. Celine by sourced genes from microalgae, oomycete or lower plant moss, producing levels of EPA/DHA equivalent to those in marine fish oils, specifically in the prototype line DHA2015.1 (DHA1) accumulating over 25% n-3 LC-PUFAs (FIG. 1b) (Han et al., 2020). We also carried out DHA1 field trials in UK, USA and Canada, and demonstrated the n-3 LC-PUFAs trait was stable and robust in distinct geographical locations and agricultural environments (Han et al., 2020). In parallel, salmon feeding trials and human dietary studies using DHA1 seeds oils both showed that these recombinant plant-based oils have equivalent health benefits and can represent an effective replacement for marine sourced fish oils, confirming their potential for commercial development (Betancor et al., 2018; West et al., 2019).

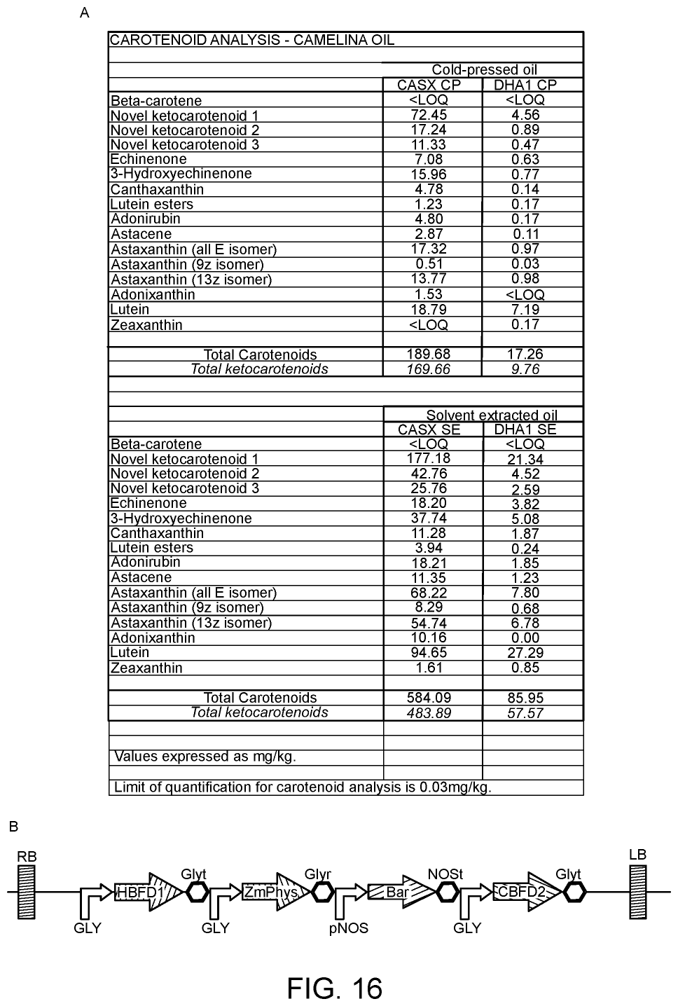

We have further engineered a Camelina line to express astaxanthin, in addition to high levels of EPA and DHA. As shown in FIG. 16, the total amount of astaxanthin in solvent-extracted oil was 131.25 mg/kg. This is higher than the level of astaxanthin produced in land plants previously. This has never been achieved before. Advantageously, the engineered Camelina line is able to reduce the content of beta-carotene, and thereby lead to a higher production of astaxanthin.

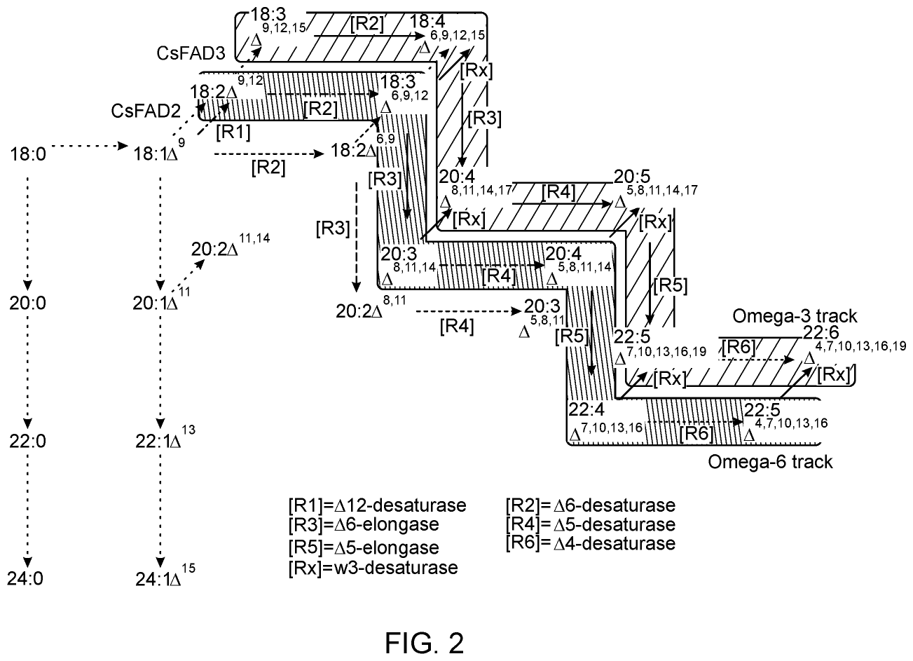

To further improve DHA1 line n-3 LC-PUFAs level, we hypothesize that increasing C18 precursor ALA can further stimulate EPA/DHA accumulation, based on our observation that ALA, as opposed to LA, is the endogenous fatty acid which is metabolised to produce these longer chain fatty acids (Han et al., 2020). Thus, a possible route to increasing the levels of EPA and DHA in our recombinant Camelina is to increase the levels of the substrate pool of ALA. Steps to directly achieve this are already contained within the DHA2015.1 construct, with the presence of D12-desaturase to drive the flux of fatty acids into PUFA biosynthesis (FIG. 1; FIG. 2). However, as a less obvious approach, we decided to test the use of a line of gene-edited FAE1 mutants of Camelina, which have shown altered seed fatty acid compositions, in part because euricic acid is unhealthy in a human diets and this would be seen as a negative for the use of the omega-3 oil described herein in human nutraceutical applications. The substrate and products of FAE1 are not known to be part of the canonical LC-PUFA biosynthetic pathway. In Camelina FAE1 functions to sequentially elongate oleic acid (OA; 18:1Δ⁹) to gondoic acid 20:1Δ¹¹ and then erucic acid 22:1Δ¹³ Thus, this pathway is in competition with the endogenous FAD2 Δ12 desaturase which desaturates OA to produce linoleic acid (LA; 18:2Δ^{9,12}), which then was further desaturated by the w3 desaturase to produce ALA. To completely ablate the activity of FAE1 requires the targeted disruption of all three homelogoues (FAE1-A, FAE1-B, and FAE1-C) present in the hexaploid Camelina cv. Suneson. By using CRISPR/cas9 technology to specifically disrupt all homeologues of FAE1, the ALA level was increased from 36.9% in wildtype (WT) to 47.3% in fae1 mutant (Ozseyhan et al., 2018)—this is in addition to the complete ablation of erucic acid, a fatty acid which is considered undesirable above a modest threshold (5% of total oil) in human foodstuffs.

Accordingly, in one aspect of the invention, there is provided a recombinant plant, part thereof or plant cell, wherein the plant, part thereof or cell expresses a nucleic acid construct comprising at least one nucleic acid sequence encoding at least a Δ6-elongase, a Δ5-desaturase and a Δ6-desaturase, and wherein the plant has a decreased very long chain fatty acid (VLCFA) content compared to a wild-type or control plant. In another aspect of the invention, there is provided a recombinant plant, part thereof or plant cell, wherein the plant, part thereof or cell expresses at least one nucleic acid sequence encoding at least a Δ6-elongase, a Δ5-desaturase and a Δ6-desaturase, and wherein the plant has a decreased very long chain fatty acid (VLCFA) content compared to a wild-type or control plant.

In another aspect of the invention, there is provided a recombinant plant, part thereof or plant cell, wherein the plant, part thereof or plant cell comprises at least one nucleic acid sequence encoding a Δ6-elongase, a Δ5-desaturase, Δ6-desaturase, a hydroxy-beta-ring 4-dehydrogenase (HBFD) and a Keto2. In a further embodiment, the plant, part thereof or plant cell further comprises a nucleic acid sequence encoding phytoene synthase. In an even further embodiment, the plant, part thereof or plant cell has a decreased very long chain fatty acid (VLCFA) content compared to a wild-type or control plant.

In a further embodiment, the plant, part thereof or cell comprises at least one mutation in a gene encoding an enzyme involved in the synthesis of VLCFAs, wherein mutation of the gene encoding the enzyme involved in the synthesis of VLCFAs leads to a decreased VLCFA content. In some embodiments, the plant comprises at least one mutation in a gene encoding an enzyme involved in the channelling of fatty acids between different lipids, such as diacylglycerol O-acyltransferase (DGAT), phospholipid:diacylglycerol acyltransferase (PDAT), carnitine palmitoyltransferase (CPT) and/or phosphatidylcholine diacylglycerol cholinephosphotransferase (PDCT).

More preferably, the plant comprises at least one mutation in a gene encoding fatty acid elongase 1 (FAE1). As described above, FAE1 catalyses the first condensation step in the elongation of VLCFAs and is thus a key gene in the production of GA. In a preferred embodiment, the sequence of FAE1 comprises or consists of SEQ ID NO: 28 and in one embodiment, may encode a FAE1 polypeptide as defined in SEQ ID NO: 31 (FAE1-A; Csa11g007400.1); or SEQ ID NO: 29 and in one embodiment may encode a FAE1 polypeptide as defined in SEQ ID NO: 32 (FAE1-B; Csa10g007610.1); or SEQ ID NO: 30 and in one embodiment may encode a FAE1 polypeptide as defined in SEQ ID NO: 33(FAE1-C; Csa12g009060.1). Also included in the scope of the invention is a homologue or variant thereof of any of these sequences.

The term homolog (or “homologue”), as used herein, also designates FAE1 gene orthologue from other plant species. A homolog may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the nucleic acid sequences as shown by SEQ ID NOs: 28, 29 or 30 In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%. Examples of FAE1 homologues are provided in SEQ ID NO: 28, 29 or 30 Functional variants of these homologues are also included within the scope of the invention. In one embodiment, where the homologue is B. napus, the homologue comprises a sequence as defined in NC_027764.2 (https://www.ncbi.nlm.nih.gov/gene/106361027).

By “at least one mutation” is meant that where the FAE1 gene is present as more than one copy or homoeologue (with the same or slightly different sequence) there is at least one mutation in at least one gene. Preferably, all genes are mutated. For example, Camelina contains three FAE1 genes: FAE1-A, FAE1-B and FAE1-C, which are more than 96% identical.

More preferably the mutation is a homozygous loss-of-function mutation. In one embodiment, the mutation in the nucleic acid sequence encoding FAE1 may be selected from one of the following mutation types:
1. a “missense mutation”, which is a change in the nucleic acid sequence (e.g. a change in one or more nucleotides) that results in the substitution of one amino acid for another amino acid (also known as a nonsynonymous substitution);
2. a “nonsense mutation” or “STOP codon mutation”, which is a change in the nucleic acid sequence that results in the introduction of a premature STOP codon and, thus, the termination of translation (resulting in a truncated protein); in plants, the translation stop codons may be selected from “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation.
3. an “insertion mutation” of one or more nucleotides or one or more amino acids, due to one or more codons having been added in the coding sequence of the nucleic acid;
4. a “deletion mutation” of one or more nucleotides or of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides.
6. a “splice site” mutation, which is a mutation that results in the insertion, deletion or substitution of a nucleotide at the site of splicing.

In a preferred embodiment, the mutation is a deletion or substitution of one or more bases. In a further preferred embodiment, the mutation is introduced using mutagenesis or targeted genome editing.

Targeted genome modification or targeted genome editing is a genome engineering technique that uses targeted DNA double-strand breaks (DSBs) to stimulate genome editing through homologous recombination (HR)-mediated recombination events.

In a preferred embodiment, the targeted genome editing technique is CRISPR. The use of this technology in genome editing is well described in the art, for example in U.S. Pat. No. 8,697,359 and references cited therein. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-Ill) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. Codon optimized versions of Cas9, which is originally from the bacterium Streptococcus pyogenes, can also be used to increase efficiency. Cas9 orthologues may also be used, such as Staphylococcus aureus (SaCas9) or Streptococcus thermophilus (StCas9).

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase Ill promoters, such as U6 and U3. Accordingly, using techniques known in the art, such as https://chopchop.cbu.uib.no/ it is possible to design sgRNA molecules that targets the FAE1 gene as described herein.

Alternatively, Cpf1, which is another Cas protein, can be used as the endonuclease. Cpf1 differs from Cas9 in several ways: Cpf1 requires a T-rich PAM sequence (TTTV) for target recognition, Cpf1 does not require a tracrRNA, and as such only crRNA is required unlike Cas9 and the Cpf1-cleavage site is located distal and downstream to the PAM sequence in the protospacer sequence (Li et al., 2017). Furthermore, after identification of the PAM motif, Cpf1 introduces a sticky-end-like DNA double-stranded break with several nucleotides of overhang. As such, the CRISPR/Cpf1 system consists of a Cpf1 enzyme and a crRNA.

Cas9 and Cpf1 expression plasmids for use in the methods of the invention can be constructed as described in the art. Cas9 or Cpf1 and the one or more sgRNA molecule may be delivered as separate or as a single construct. Where separate constructs are used for the delivery of the CRISPR enzyme (i.e. Cas9 or Cpf1) and the sgRNA molecule (s), the promoters used to drive expression of the CRISPR enzyme/sgRNA molecule may be the same or different. In one embodiment, RNA polymerase (Pol) Il-dependent promoters can be used to drive expression of the CRISPR enzyme. In another embodiment, Pol Ill-dependent promoters, such as U6 or U3, can be used to drive expression of the sgRNA.

In one embodiment, the method uses a sgRNA to introduce a targeted SNP or mutation, in particular one of the substitutions described herein, into FAE1 gene. As explained below, the introduction of a template DNA strand, following a sgRNA-mediated snip in the double-stranded DNA, can be used to produce a specific targeted mutation (i.e. a SNP) in the gene using homology directed repair. In another example, sgRNA (for example, as described herein) can be used with a modified Cas9 protein, such as nickase Cas9 or nCas9 or a “dead” Cas9 (dCas9) fused to a “Base Editor”—such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made (Kim et al., 2017; Gaudelli et al. 2017).

The genome editing constructs may be introduced into a plant cell using any suitable method known to the skilled person. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation, biolistic bombardment or microinjection.

In one example, one or more mutation may be introduced into one, preferably all FAE1 genes using the CRISPR constructs described in Ozseyhan et al. 2018, which is incorporated herein by reference. As described in Ozseyhan et al. 2018, sgRNA sequences were generated with the following sequences: g-RNA-F 5′-ATTGTTGGAGATGGGAATAGAAG-3′ (SEQ ID NO: 26) and g-RNA-R 5′-AAACCTTCTATTCCCATCTCCAA-3′ (SEQ ID NO: 27). These sgRNA sequences were synthesised as two complementary oligonucleotides and inserted into the pHEE401 E transformation vector. The sgRNA-Cas9 cassette was sub-cloned into the pBinGlyRed2 vector to use the DsRed as the selection marker. This construct was transformed into Camelina using the Agrobacterium-mediated vacuum infiltration method to obtain recombinant seeds.

In an alternative embodiment, the expression of one or more FAE1 genes can be accomplished by introducing a silencing construct such as RNAi into the plant. Accordingly, in an alternative embodiment, the plant expresses a nucleic acid construct encoding a silencing construct against FAE1, such as a RNAi. Such constructs can be introduced using the transformation methods described herein. In one embodiment, the siNA may include, short interfering RNA (siRNA), double-stranded RNA (dsRNA), micro-RNA (miRNA), antagomirs and short hairpin RNA (shRNA) capable of mediating RNA interference.

Alternatively, more conventional mutagenesis methods can be used to introduce at least one mutation into at least one FAE1 gene. These methods include both physical and chemical mutagenesis. A skilled person will know further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

In one example, mutagenesis is physical mutagenesis, such as application of ultraviolet radiation, X-rays, gamma rays, fast or thermal neutrons or protons. In another example, by chemical mutagenesis is meant mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. In a specific example the one or more mutation may be introduced using induced local lesions in genomes (TILLING), reviewed in Henikoff et al, 2004. In either embodiment, the targeted population can then be screened to identify a FAE1 mutant.

In a further alternative embodiment, insertional mutagenesis is used, for example using T-DNA mutagenesis (which inserts pieces of the T-DNA from the Agrobacterium tumefaciens T-Plasmid into DNA causing either loss of gene function or gain of gene function mutations), site-directed nucleases (SDNs) or transposons as a mutagen. Insertional mutagenesis is an alternative means of disrupting gene function and is based on the insertion of foreign DNA into the gene of interest (see Krysan et al, The Plant Cell, Vol. 11, 2283-2290, December 1999).

In one embodiment, the recombinant plant, part thereof or plant cell comprises a nucleic acid sequence encoding a Δ6-elongase, a Δ5-desaturase, a Δ6-desaturase, a w3-desaturase, a Δ12-desaturase, a Δ5-elongase and a Δ4-desaturase. In another embodiment, the recombinant plant, part thereof or plant expresses at least one nucleic acid construct that comprises a nucleic acid sequence encoding a Δ6-elongase, a Δ5-desaturase, a Δ6-desaturase, a w3-desaturase, a Δ12-desaturase, a Δ5-elongase and a Δ4-desaturase. Preferably, the plant, part thereof or plant cell or nucleic acid construct does not comprise a nucleic acid sequence encoding any other enzyme involved in the synthesis of a LC-PUFA. In other words, the construct is a 7-gene construct. As used herein, the construct may be referred to as “DHA2015.1” or “DHA1”.

In a preferred embodiment, the Δ6-elongase is derived from Physcomitrella patens. More preferably, the nucleic acid sequence encodes a Δ6-elongase derived from Physcomitrella patens as defined in SEQ ID NO: 4 or a functional variant thereof. In a further embodiment, the nucleic acid encoding a Δ6-elongase derived from Physcomitrella patens comprises SEQ ID NO: 3 or a functional variant thereof.

In a further preferred embodiment, the Δ5-desaturase is derived from Thraustochytrium. More preferably, the nucleic acid sequence encodes a Δ5-desaturase derived from Thraustochytrium as defined in SEQ ID NO: 6 or a functional variant thereof. In a further preferred embodiment, the nucleic acid sequence encoding a Δ5-desaturase derived from Thraustochytrium comprises SEQ ID NO: 5 or a functional variant thereof.

In another embodiment, the Δ6-desaturase is derived from Ostreococcus tauri. More preferably, the nucleic acid sequence encodes a Δ6-desaturase derived from Ostreococcus tauri as defined in SEQ ID NO: 2 or a functional variant thereof. In a further preferred embodiment, the nucleic acid sequence encoding a Δ6-desaturase derived from Ostreococcus tauri comprises SEQ ID NO: 1 or a functional variant thereof.

In another embodiment, the w3-desaturase is derived from Phytophora infestans. In a further embodiment, the nucleic acid sequence encodes a w3-desaturase derived from Phytophora infestans as defined in SEQ ID NO: 12 or a functional variant thereof. More preferably, the w3-desaturase derived from Phytophora infestans comprises a nucleotide sequence as defined in SEQ ID NO: 11 or a functional variant thereof.

In another embodiment, the Δ12-desaturase is derived from Phytophora sojae. Preferably, the nucleic acid encodes a Δ12-desaturase as defined in SEQ ID NO: 10 or a functional variant thereof. In a further embodiment, the nucleic acid encoding a Δ12-desaturase comprises SEQ ID NO: 11 or a functional variant thereof.

In another embodiment, the construct further comprises Δ5-elongase derived from Ostreococcus tauri. More preferably, the nucleic acid sequence encodes a Δ5-elongase as defined in SEQ ID NO: 8 or a functional variant thereof. In a further preferred embodiment, the Δ5-elongase comprises a nucleotide sequence as defined in SEQ ID NO: 7 or a functional variant thereof.

In another embodiment, the construct further comprises a Δ4-desaturase derived from Ostreococcus RCC809. More preferably, the nucleic acid sequence encodes a Δ4-desaturase as defined in SEQ ID NO: 18 or a functional variant thereof. In a further preferred embodiment, the Δ4-desaturase comprises a nucleotide sequence as defined in SEQ ID NO: 17 or 25 or a functional variant thereof.

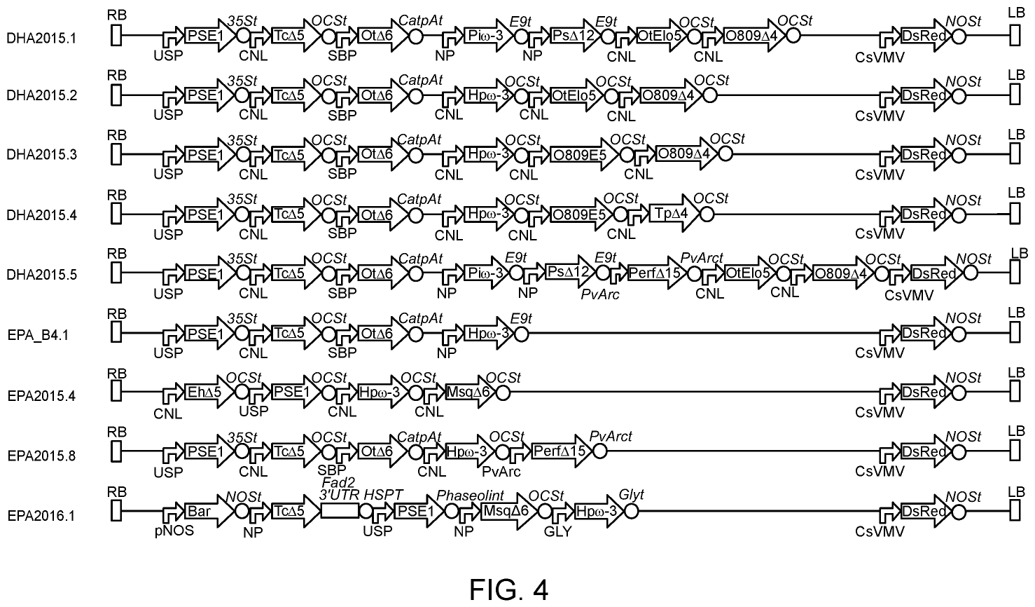

In one embodiment, the nucleic acid sequences of the DHA1 construct are operably linked to one or more regulatory sequences. In a preferred embodiment, the Δ6-elongase is operably linked to USP (unknown seed protein seed-specific promoter); the Δ5-desaturase is operably linked to CNL (25 seed storage protein (Conlinin) promoter); the Δ6-desaturase is operably linked to SBP (sucrose-binding protein promoter (a seed specific promoter); the w3-desaturase is operably linked to NP (Napin seed-specific promoter); Δ12-desaturase is operably linked to NP; Δ5-elongase is operably linked to CNL and the Δ4-desaturase is operably linked to CNL. In a further embodiment, the nucleic acid construct DHA1 preferably comprises one or more termination sequences as described in FIG. 4.

In another embodiment, the recombinant plant, part thereof or plant cell comprises a nucleic acid sequence encoding a Δ6-elongase, a Δ5-desaturase, Δ6-desaturase, a hydroxy-beta-ring 4-dehydrogenase (HBFD1) and a Keto2. In one embodiment, the recombinant plant, part thereof or plant cell may express a first construct that is DHA1 and a second construct that comprises a nucleic acid sequence encoding HBFD1 and a Keto2 operably linked to a regulatory sequence. The second construct may optionally further comprise a nucleic acid sequence encoding for phytoene synthase. In a further embodiment, the regulatory sequence is the Glycinin promoter. The second construct may also comprise a termination sequence, where the termination sequence is a Glycinin termination sequence.

Alternatively, in another embodiment, the recombinant plant, part thereof or plant cell expresses a nucleic acid construct comprising nucleic acid sequences encoding a Δ6-elongase, a Δ5-desaturase, Δ6-desaturase, a hydroxy-beta-ring 4-dehydrogenase (HBFD1) and a Keto2 operably linked to a regulatory sequence. The construct may optionally further comprise a nucleic acid sequence encoding for phytoene synthase.

In one embodiment, HBFD1 is derived from Adonis aestivalis. More preferably the nucleic acid sequence encodes a HBFD1 as defined in SEQ ID NO: 34 or a functional variant thereof. In a further preferred embodiment, the HBFD1comprises a nucleotide sequence as defined in SEQ ID NO: 37 or a functional variant thereof.

In one embodiment, Keto2 is derived from Adonis aestivalis. More preferably the nucleic acid sequence encodes a Keto2 as defined in SEQ ID NO: 35 or a functional variant thereof. In a further preferred embodiment, the HBFD1comprises a nucleotide sequence as defined in SEQ ID NO: 38 or a functional variant thereof.

In one embodiment, phytoene synthase is derived from Zea mays. More preferably the nucleic acid sequence encodes a phytoene synthase as defined in SEQ ID NO: 36 or a functional variant thereof. In a further preferred embodiment, the phytoene synthase comprises a nucleotide sequence as defined in SEQ ID NO: 39 or a functional variant thereof.

As used herein “DHA1fae1” refers to lines expressing both the DHA1 construct and a mutation in fae1.

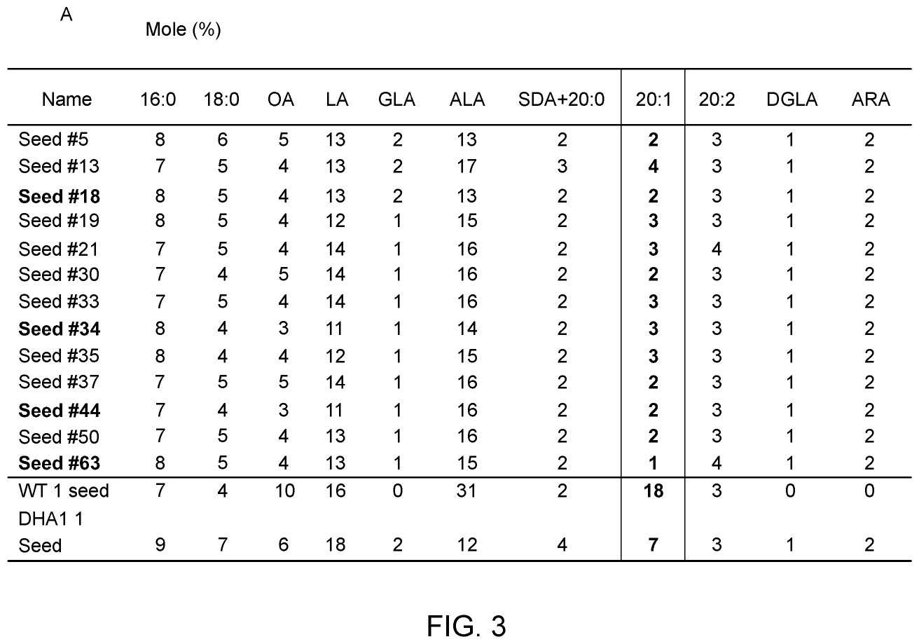

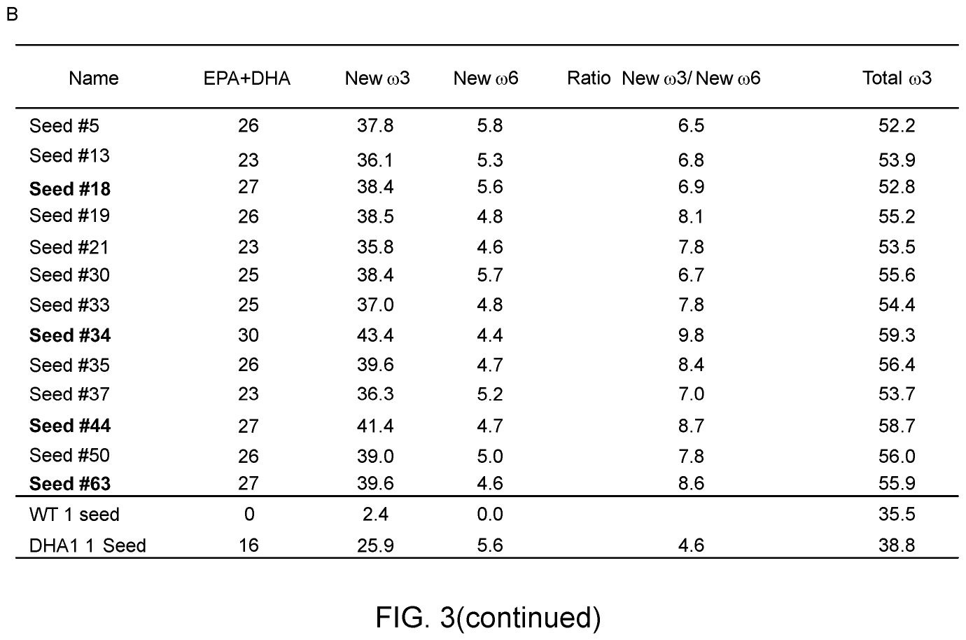

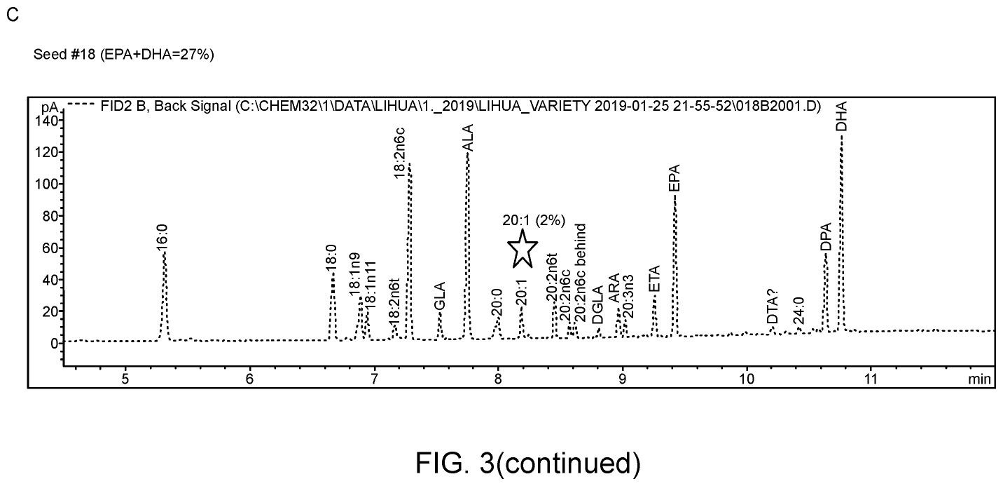

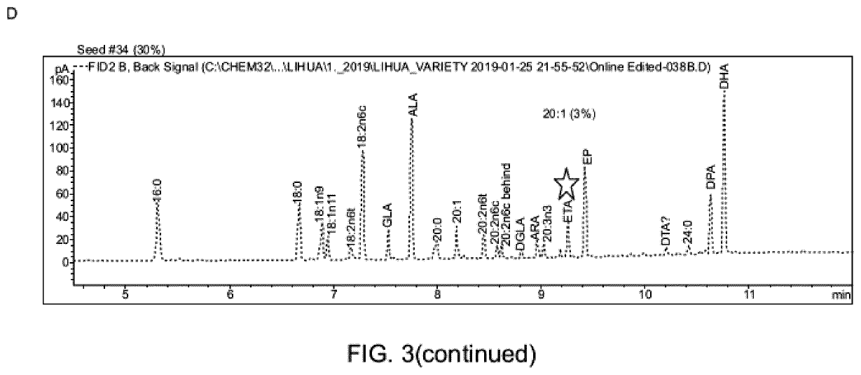

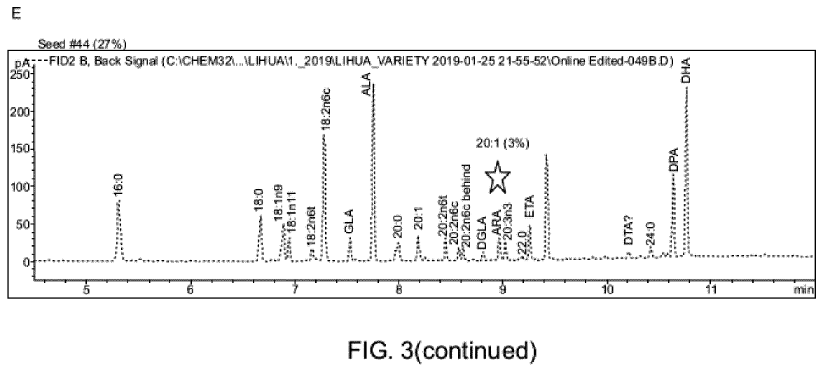

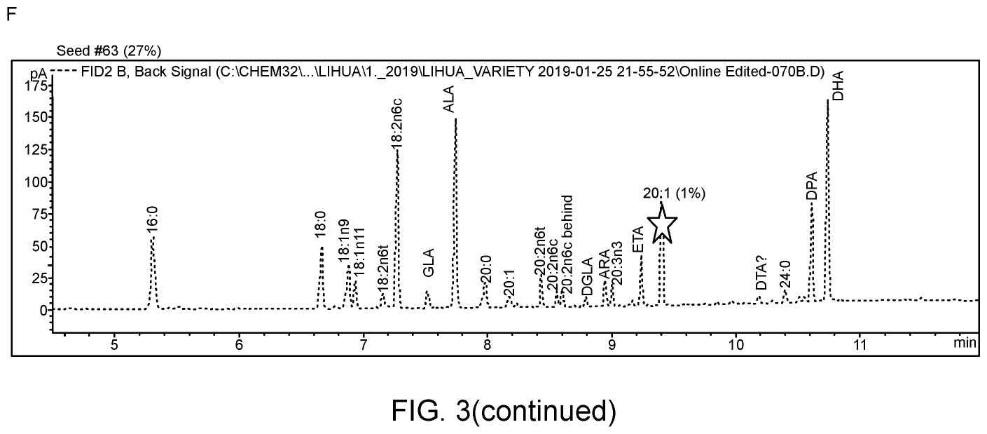

As shown in FIG. 2 the, total C20+n-3 fatty acids contents including ETA, EPA, DPA, DHA were increased by 5.5% from 27.5% in DHA1 to 33.0% in DHA1fae1. Furthermore, the total n-3 content was 47.5% in DHA1 and 58.2% in DHA1fae1, whereas the total n-6 content was reduced from 23.6% to 20.2% respectively. Therefore, the ratio of total (n-3/n-6) was 2.0 in DHA1 and 2.9 in DHA1fae1, which is also a significant increase compared with 2.2 in the fae1 mutant and 1.7 in both WT Celine and WT Suneson lines, indicating that the DHA1fae1 fatty acids have an even better health benefit than those of DHA1, fae1 and WTs. The beneficial effect of combining DHA1 expressing plants and fae1 mutants can be further seen in FIG. 3.

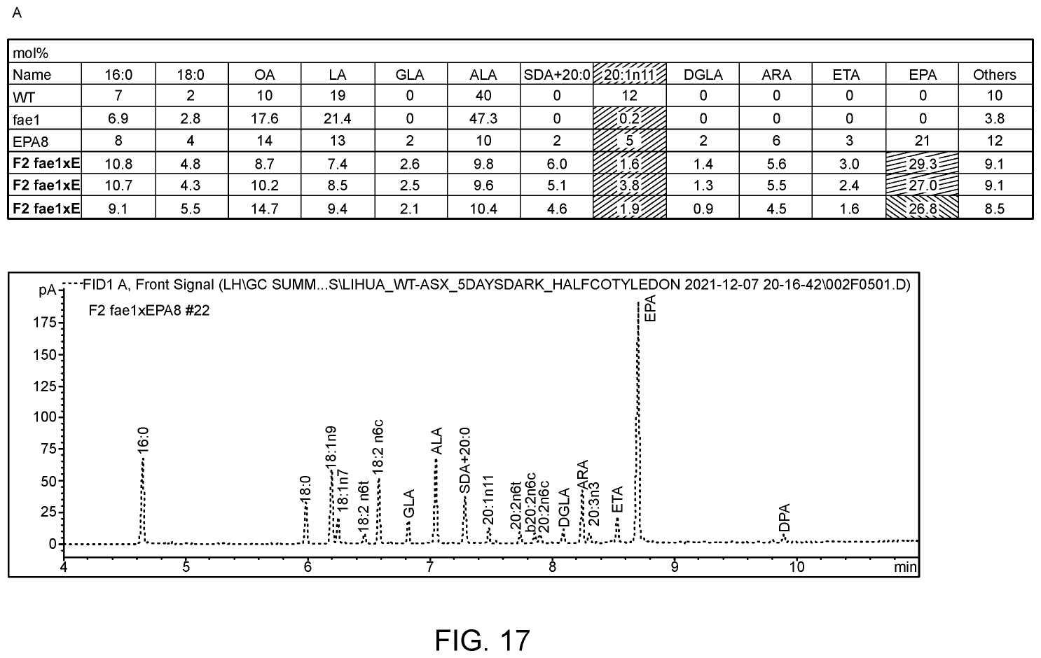

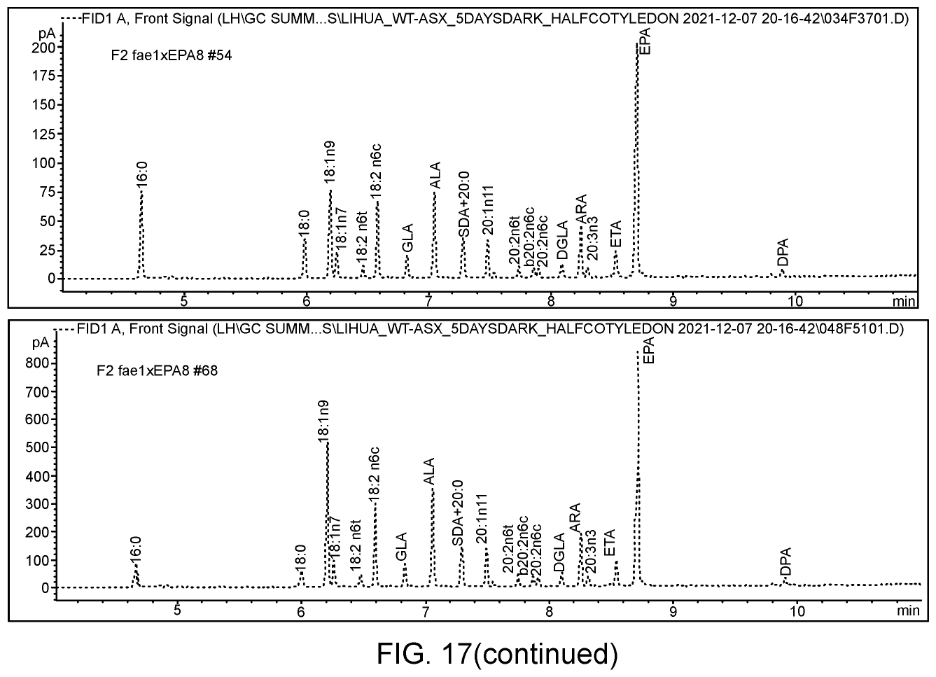

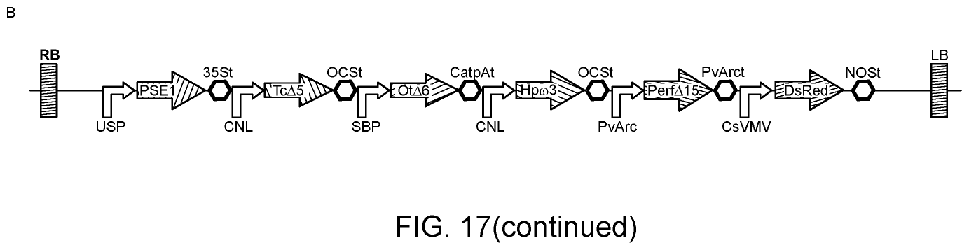

In an alternative embodiment, the recombinant plant, part thereof or plant cell expresses a nucleic acid construct comprising a nucleic acid sequence encoding a Δ6-elongase, a Δ5-desaturase, a Δ6-desaturase, a w3-desaturase and a Δ15-desaturase. Preferably, the nucleic acid construct does not comprise a nucleic acid sequence encoding any other enzyme involved in the synthesis of a LC-PUFA. In other words, the nucleic acid construct comprises a nucleic acid sequence encoding only a Δ6-elongase, a Δ5-desaturase, a Δ6-desaturase, a w3-desaturase and a Δ15-desaturase (it is a 5 gene construct). This construct is referred to herein as “EPA2015.8” or “EPA8”. Expression of this construct may be in addition to the expression of nucleic acid sequences encoding HBFD1, Keto2 and optionally phytoene synthase, and/or a mutation in fae1. An F2 generation that is the result of a cross between plants expressing EPA8 and plants with a mutation in fae1 is shown in FIG. 17. As can be seen, expression of EPA8 in a fae1 mutant background significantly increases the level of EPA from ˜ 21% to 29.3%. This result is significant since this result closely replicates the fatty acid profile of a premium Southern Hemisphere fish oil, which is about ˜18% EPA and 12% DHA.

In one embodiment, the Δ15-desaturase is an FAD3 Δ15-desaturase. In one embodiment, the Δ15-desaturase is derived from Perilla frutescens. In another embodiment, the Δ15-desaturase is another higher plant FAD3 Δ15-desaturase, such as those derived from Camelina, Anemonastrum or Borago officinalis. More preferably, the nucleic acid sequence encodes a Δ15-desaturase as defined in SEQ ID NO: 20 or a functional variant thereof. In a further preferred embodiment, the nucleic acid sequence encoding a Δ15-desaturase comprises a nucleotide sequence as defined in SEQ ID NO: 19 or a functional variant thereof.

In another embodiment, the w3-desaturase is derived from Hyaloperonospora parasitica or Phytophora infestans. More preferably in the EPA2015.8 construct, the w3-desaturase is derived from Hyaloperonospora parasitica. In a further embodiment, the nucleic acid sequence encodes a w3-desaturase derived from Hyaloperonospora parasitica as defined in SEQ ID NO: 14 or a functional variant thereof. More preferably, the w3-desaturase derived from Hyaloperonospora parasitica comprises a nucleotide sequence as defined in SEQ ID NO: 13 or a functional variant thereof.

In a preferred embodiment, the Δ6-elongase is derived from Physcomitrellapatens. More preferably, the nucleic acid sequence encodes a Δ6-elongase derived from Physcomitrella patens as defined in SEQ ID NO: 4 or a functional variant thereof. In a further embodiment, the nucleic acid encoding a Δ6-elongase derived from Physcomitrella patens comprises SEQ ID NO: 3 or a functional variant thereof. In a further preferred embodiment, the Δ5-desaturase is derived from Thraustochytrium. More preferably, the nucleic acid sequence encodes a Δ5-desaturase derived from Thraustochytrium as defined in SEQ ID NO: 6 or a functional variant thereof. In a further preferred embodiment, the nucleic acid sequence encoding a Δ5-desaturase derived from Thraustochytrium comprises SEQ ID NO: 5 or a functional variant thereof. In another embodiment, the Δ6-desaturase is derived from Ostreococcus tauri. More preferably, the nucleic acid sequence encodes a Δ6-desaturase derived from Ostreococcus tauri as defined in SEQ ID NO: 2 or a functional variant thereof. In a further preferred embodiment, the nucleic acid sequence encoding a Δ6-desaturase derived from Ostreococcus tauri comprises SEQ ID NO: 1 or a functional variant thereof.

In one embodiment, the nucleic acid sequences of the EPA2015.8 construct are operably linked to one or more regulatory sequences. In a preferred embodiment, the Δ6-elongase is operably linked to USP (unknown seed protein seed-specific promoter); the Δ5-desaturase is operably linked to CNL (25 seed storage protein (Conlinin) promoter); the Δ6-desaturase is operably linked to SBP (sucrose-binding protein promoter (a seed specific promoter); the w3-desaturase is operably linked to CNL and Δ15-desaturase is operably linked to PvArc (Arcelin-5 storage protein promoter). In a further embodiment, the nucleic acid construct EPA2015.8 preferably comprises one or more termination sequences as described in FIG. 4.

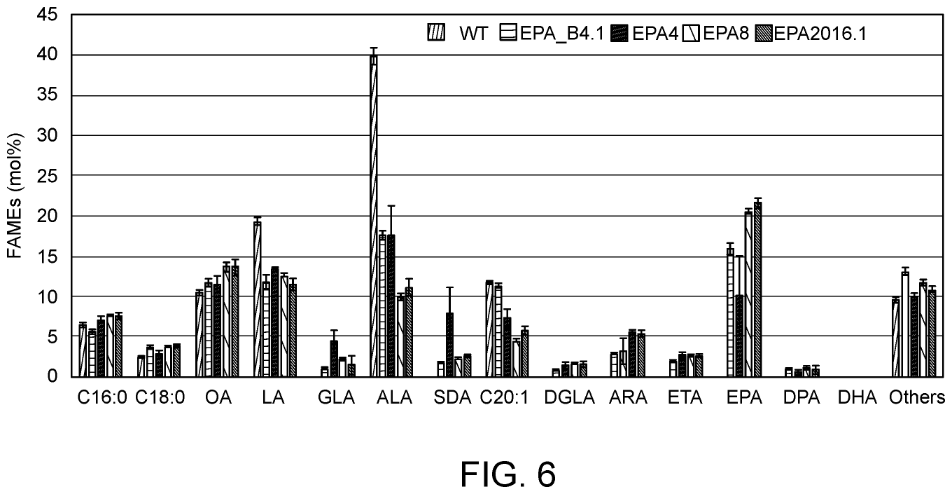

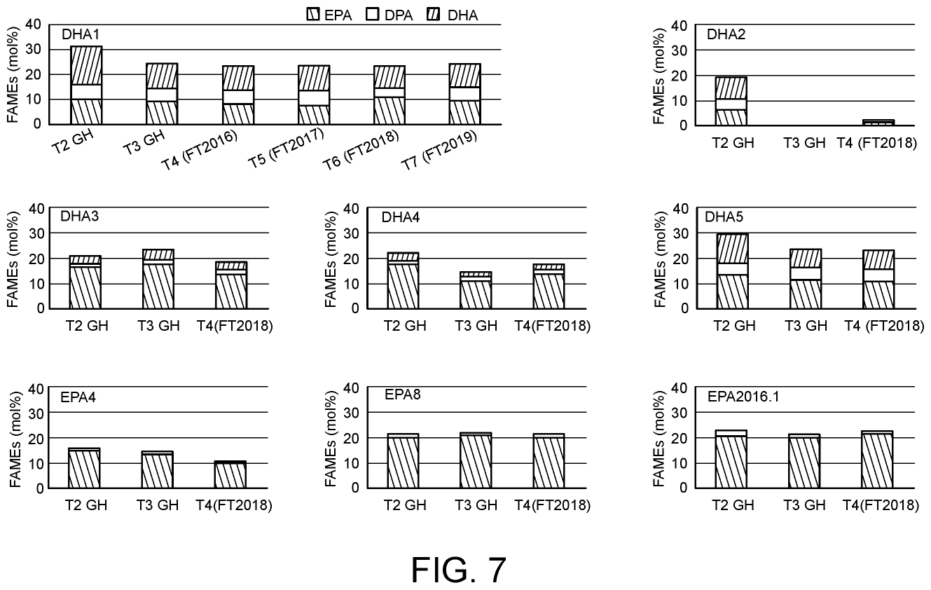

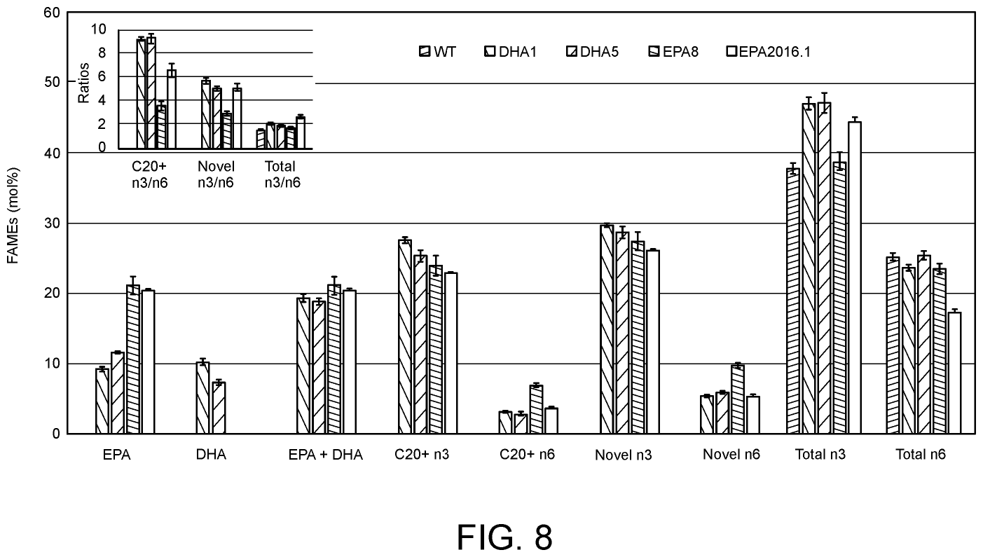

As shown in FIGS. 6 to 12 expression of the EPA8 construct in plants led to decrease in LA content (compared to wild-type) showing an increase in flux through the omega-3 pathway. More importantly, expression of the EPA8 construct led to a significant increase in EPA levels over EPA_B4.1 in both field trials and in greenhouse trials (FIGS. 6, 8, 10 and 12). In each trial, levels of EPA were increased to at least 20% or more (mol %) of the total fatty acid content. As shown in FIG. 7, this increase was maintained over three generations (from T2 to T3 and T4). As further shown in FIG. 8, expression of the EPA8 construct also significantly increased the total amount of EPA and DHA, the amount of C20+ omega-3 LCFAs (i.e. ETA, EPA, DPA and DHA) as well as the total amount of omega-3 LCFAs, while decreasing total omega-6 LCFAs compared to wild-type plants.

In a further alternative embodiment, the nucleic acid construct comprises a nucleic acid sequence encoding a Δ6-elongase, a Δ5-desaturase, a Δ6-desaturase, a w3-desaturase, a Δ12-desaturase, a Δ15-desaturase, a Δ5-elongase and a Δ4-desaturase. Again, preferably, the nucleic acid construct does not comprise a nucleic acid sequence encoding any other enzyme involved in the synthesis of a LC-PUFA. In other words, the construct is a 8-gene construct. As used herein, the construct may be referred to as “DHA2015.5” or “DHA5”.

In a preferred embodiment, the Δ6-elongase is derived from Physcomitrellapatens. More preferably, the nucleic acid sequence encodes a Δ6-elongase derived from Physcomitrella patens as defined in SEQ ID NO: 4 or a functional variant thereof. In a further embodiment, the nucleic acid encoding a Δ6-elongase derived from Physcomitrella patens comprises SEQ ID NO: 3 or a functional variant thereof.

In a further preferred embodiment, the Δ5-desaturase is derived from Thraustochytrium. More preferably, the nucleic acid sequence encodes a Δ5-desaturase derived from Thraustochytrium as defined in SEQ ID NO: 6 or a functional variant thereof. In a further preferred embodiment, the nucleic acid sequence encoding a Δ5-desaturase derived from Thraustochytrium comprises SEQ ID NO: 5 or a functional variant thereof.

In another embodiment, the Δ6-desaturase is derived from Ostreococcus tauri. More preferably, the nucleic acid sequence encodes a Δ6-desaturase derived from Ostreococcus tauri as defined in SEQ ID NO: 2 or a functional variant thereof. In a further preferred embodiment, the nucleic acid sequence encoding a Δ6-desaturase derived from Ostreococcus tauri comprises SEQ ID NO: 1 or a functional variant thereof.

In another embodiment, the w3-desaturase is derived from Hyaloperonospora parasitica or Phytophora infestans. More preferably in the DHA5 construct, the w3-desaturase is derived from Phytophora infestans. In a further embodiment, the nucleic acid sequence encodes a w3-desaturase derived from Phytophora infestans as defined in SEQ ID NO: 12 or a functional variant thereof. More preferably, the w3-desaturase derived from Phytophora infestans comprises a nucleotide sequence as defined in SEQ ID NO: 11 or a functional variant thereof.

In another embodiment, the Δ12-desaturase is derived from Phytophora sojae. Preferably, the nucleic acid encodes a Δ12-desaturase as defined in SEQ ID NO: 10 or a functional variant thereof. In a further embodiment, the nucleic acid encoding a Δ12-desaturase comprises SEQ ID NO: 11 or a functional variant thereof.

In one embodiment, the Δ15-desaturase is derived from Perilla frutescens. In another embodiment, the Δ15-desaturase is another higher plant FAD3 Δ15-desaturase, such as those derived from Camelina, Anemonastrum or Borago officinalis. More preferably, the nucleic acid sequence encodes a Δ15-desaturase as defined in SEQ ID NO: 20 or a functional variant thereof. In a further preferred embodiment, the nucleic acid sequence encoding a Δ15-desaturase comprises a nucleotide sequence as defined in SEQ ID NO: 19 or a functional variant thereof.

In another embodiment, the construct further comprises Δ5-elongase derived from Ostreococcus tauri. More preferably, the nucleic acid sequence encodes a Δ5-elongase as defined in SEQ ID NO: 8 or a functional variant thereof. In a further preferred embodiment, the Δ5-elongase comprises a nucleotide sequence as defined in SEQ ID NO: 7 or a functional variant thereof.

In another embodiment, the construct further comprises a Δ4-desaturase derived from Ostreococcus RCC809. More preferably, the nucleic acid sequence encodes a Δ4-desaturase as defined in SEQ ID NO: 18 or a functional variant thereof. In a further preferred embodiment, the Δ4-desaturase comprises a nucleotide sequence as defined in SEQ ID NO: 17 or 25 or a functional variant thereof.

In one embodiment, the nucleic acid sequences of the DHA5 construct are operably linked to one or more regulatory sequences. In a preferred embodiment, the Δ6-elongase is operably linked to USP (unknown seed protein seed-specific promoter); the Δ5-desaturase is operably linked to CNL (25 seed storage protein (Conlinin) promoter); the Δ6-desaturase is operably linked to SBP (sucrose-binding protein promoter (a seed specific promoter); the w3-desaturase is operably linked to NP (Napin seed-specific promoter); Δ12-desaturase is operably linked to NP; Δ15-desaturase is operably linked to PvArc (Arcelin-5 storage protein promoter), Δ5-elongase is operably linked to CNL and the Δ4-desaturase is operably linked to CNL. In a further embodiment, the nucleic acid construct DHA5 preferably comprises one or more termination sequences as described in FIG. 4.

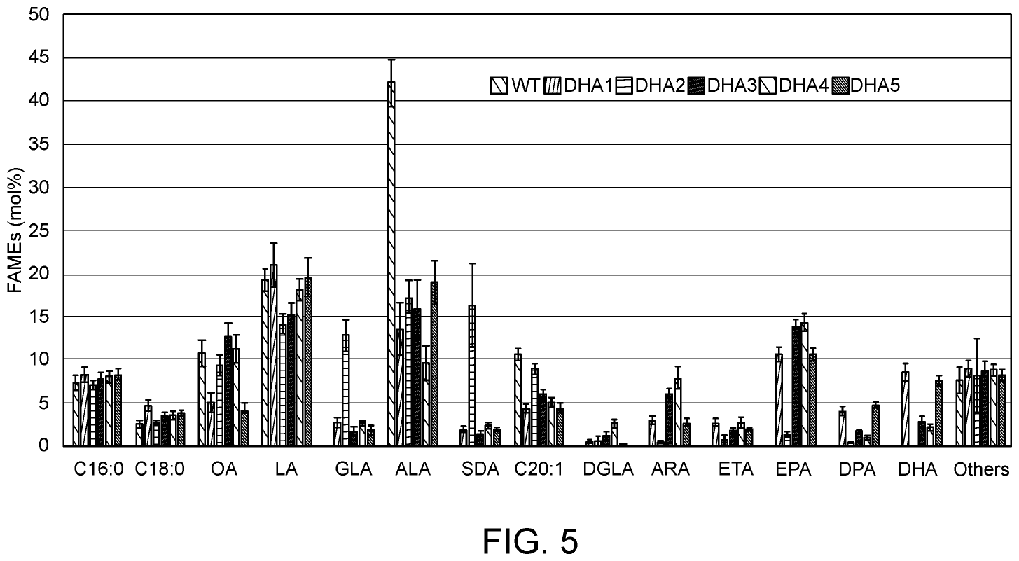

As shown in FIGS. 6 to 12, expression of the DHA5 construct in plants led to decrease in LA content (compared to wild-type) showing an increase in flux through the omega-3 pathway, as well as a decrease in production of the undesirable omega-6 intermediate, GA (FIG. 5). More importantly, expression of the DHA5 construct led to equivalent or increased levels of ALA, EPA, DPA and/or DHA levels over DHA1 (DHA2015.1) in both field trials and in greenhouse trials (FIGS. 5, 8, 9 and 11). In particular, as shown in FIGS. 5, 8 and 9 DHA5 led to a significant increase in ALA and EPA levels compared to DHA1, as well as commercially useful levels of DPA and DHA. In each trial, levels of EPA were consistently increased to at least 10%, while DPA was increased to at least 5% and DHA to at least 15% or more (mol %) of the total fatty acid content. As shown in FIG. 7, this increase was maintained over three generations (from T2 to T3 and T4).

As further shown in FIG. 8, expression of the DHA5 construct also significantly increased the total amount of EPA and DHA, the amount of C20+ omega-3 LCFAs (i.e. ETA, EPA, DPA and DHA) as well as the total amount of omega-3 LCFAs. As Camelina plants are already rich in ALA and endogenously express a Δ15-desaturase, it was unexpected that the addition of PerfD15 could increase levels of omega-3 fatty acids (and in particular ALA and EPA) over the DHA1 construct.

In another alternative embodiment, the nucleic acid construct comprises a nucleic acid sequence encoding a Δ6-elongase, a Δ5-desaturase, a Δ6-desaturase and a ω3-desaturase, wherein the Δ6-desaturase is derived from Mantoniella squamata. Again, preferably, the nucleic acid construct does not comprise a nucleic acid sequence encoding any other enzyme involved in the synthesis of a LC-PUFA. In other words, the construct is a 4-gene construct. As used herein, the construct may be referred to as “EPA2016.1”.

In one embodiment, the nucleic acid sequence encodes a Δ6-desaturase derived from Mantoniella squamata as defined in SEQ ID NO: 22 or a functional variant thereof. More preferably, the nucleic acid sequence encoding a Δ6-desaturase derived from Mantoniella squamata comprises SEQ ID NO: 21 or a functional variant thereof.

In a preferred embodiment, the Δ6-elongase is derived from Physcomitrella patens. More preferably, the nucleic acid sequence encodes a Δ6-elongase derived from Physcomitrella patens as defined in SEQ ID NO: 4 or a functional variant thereof. In a further embodiment, the nucleic acid encoding a Δ6-elongase derived from Physcomitrella patens comprises SEQ ID NO: 3 or a functional variant thereof.

In a further preferred embodiment, the Δ5-desaturase is derived from Thraustochytrium. More preferably, the nucleic acid sequence encodes a Δ5-desaturase derived from Thraustochytrium as defined in SEQ ID NO: 6 or a functional variant thereof. In a further preferred embodiment, the nucleic acid sequence encoding a Δ5-desaturase derived from Thraustochytrium comprises SEQ ID NO: 5 or a functional variant thereof.

In another embodiment, the w3-desaturase is derived from Hyaloperonospora parasitica. In a further embodiment, the nucleic acid sequence encodes a w3-desaturase derived from Hyaloperonospora parasitica as defined in SEQ ID NO: 14 or a functional variant thereof. More preferably, the w3-desaturase derived from Hyaloperonospora parasitica comprises a nucleotide sequence as defined in SEQ ID NO: 13 or a functional variant thereof.

In one embodiment, the nucleic acid sequences of the EPA2016.1 construct are operably linked to one or more regulatory sequences. Preferably, the nucleic acid sequences are each operably linked to a regulatory sequence, where the regulatory sequence is selected from the unknown seed protein seed-specific promoter, the Napin seed specific promoter, the 25 seed storage protein (Conlinin) promoter, the 11S seed storage protein (Glycinin) promoter, the sucrose-binding protein promoter and the Arcelin-5 seed storage protein promoter. In a preferred embodiment, the Δ6-elongase is operably linked to USP (unknown seed protein seed-specific promoter); the Δ5-desaturase is operably linked to NP (Napin seed-specific promoter) and is flanked at the 3′ end by a UTR (Untranslated Region); the w3-desaturase is operably linked to GLY (11S seed storage protein (Glycinin) promoter); and the Δ6-desaturase is operably linked to NP. In a further embodiment, the nucleic acid construct EPA2016.1 preferably comprises one or more termination sequences as described in FIG. 4.

As shown in FIGS. 6 to 12, expression of the EPA2016.1 construct in plants led to decrease in LA content (compared to wild-type) showing an increase in flux through the omega-3 pathway. More importantly, expression of the EPA2016.1 construct led to a significant increase in EPA levels over EPA_B4.1 in both field trials and in greenhouse trials (FIGS. 6, 8, 10 and 12). On possible explanation for the significant increase in EPA levels could be the addition of the UTR sequences, which was added 3′ of the Δ5-desaturase to increase the transcript stability and also ensure correct transcriptional termination. In each trial, levels of EPA were increased to at least 20% or more (mol %) of the total fatty acid content. As shown in FIG. 7, this increase was maintained over three generations (from T2 to T3 and T4). As further shown in FIG. 8 expression of the EPA2016.1 construct also significantly increased the total amount of EPA and DHA, the amount of C20+ omega-3 LCFAs (i.e. ETA, EPA, DPA and DHA) as well as the total amount of omega-3 LCFAs, while decreasing total omega-6 LCFAs compared to wild-type plants.

As used herein a “nucleic acid construct” is interchangeable with “expression construct” and “vector”. It is understood that a nucleic acid construct will contain all the elements required for expression of a heterologous sequence, including but not limited to, regulatory elements, such as promoters, markers and termination sequences.

In a further embodiment, the construct further comprises a nucleic acid sequence encoding resistance to at least one herbicide. In one example, the nucleic acid encodes a bar gene, which encodes a phosphinothricin acetyl transferase, which provides resistance to Class H herbicides, such as Basta^{R}, in which bialaphos is the active ingredient.

In another embodiment, any of the nucleic acid constructs of the invention are stably incorporated into the plant genome. This means that progeny plant is stably transformed with one or more of the nucleic acid constructs described herein and comprises the exogenous polynucleotide, which is heritably maintained in the plant cell. The method may include steps to verify that the construct is stably integrated. The method may also comprise the additional step of collecting seeds from the selected progeny plant. In one example, probes and primers corresponding to and capable of hybridisation to one or more of the nucleic acid sequences within the described constructs could be used to detect incorporation of the construct into the genome.

Preferably, the plant, part thereof or plant cell has increased production of omega-3 LC-PUFAs.

In one embodiment, the plant, part thereof or plant cell has an increased production of EPA and/or DHA, wherein EPA constitutes at least 5% (mole %) of the total fatty acid content of the plant, part thereof or plant cell and wherein DHA constitutes at least 5% (mole %) of the total fatty acid content of the plant, part thereof or plant cell; and
wherein the plant, part thereof or plant cell has a gondoic acid (GA) content of 10% (mole %) or less based on the total fatty acid content of the plant, part thereof or plant cell, and/or wherein the plant, part thereof or plant cell has a ketocarotenoid content of at least 100 mg per kg of the plant, part thereof or plant cell.

In one embodiment, the plant, part thereof or plant cell has increased production of DHA, wherein preferably the DHA content is at least 5% (mole %), more preferably 6% (mole %) or more, even more preferably 7% (mole %) or more, yet even more preferably 10% (mole %) or more, yet even more preferably 12% (mole %), and most preferably 15% (mole %) or more of the total fatty acid content of the plant, part thereof or plant cell. In a preferred embodiment, DHA constitutes between 5% and 30% (mole %), preferably between 5% and 25% (mole %), more preferably between 5% and 20% (mole %), and even more preferably between 10% and 20% (mole %) of the total fatty acid content of the plant, part thereof or plant cell.

In another an alternative or additional embodiment, the plant, part thereof or plant cell has increased production of EPA. As used herein, an “increased production of EPA” may be relative to a wild-type plant and/or a DHA1 construct. Preferably the EPA content is at least 5% (mole %), more preferably 7% (mole %) or more, even more preferably 8% (mole %) or more, yet even more preferably 9% (mole %) or more, most preferably 10% (mole %) or more of the total fatty acid content of the plant, part thereof or plant cell. In a preferred embodiment, EPA constitutes between 8% and 30% (mole %), preferably between 8% and 25% (mole %), and more preferably between 8% and 20% (mole %) of the total fatty acid content of the plant, part thereof or plant cell.

In a further embodiment, the plant, part thereof or plant cell has increased production of DPA. As used herein, an “increased production of DPA” may be relative to a wild-type plant and/or a DHA1 construct. Preferably the DPA content is at least 3% (mole %), more preferably 4% (mole %) or more, even more preferably 5% (mole %) or more, and yet even more preferably 6% (mole %) or more of the total fatty acid content of the plant, part thereof or plant cell. In a preferred embodiment, DPA constitutes between 3% and 30% (mole %), preferably between 3% and 25% (mole %), and more preferably between 3% and 20% (mole %) of the total fatty acid content of the plant, part thereof or plant cell.

In a further additional or alternative embodiment, the plant, part thereof or plant cell has decreased levels of Gondoic acid (GA). As used herein, “decreased levels of GA” may be relative to a wild-type plant and/or a DHA1 construct. Preferably the GA content is 10% (mole %) or less, more preferably 8% (mole %) or less, even more preferably 7% (mole %) or less, yet even more preferably 6% (mole %) or less, yet even more preferably 5% (mole %) or less, most preferably 4% (mole %) or less of the total fatty acid content of the plant, part thereof or plant cell. In a preferred embodiment, GA constitutes between 0.1% and 9% (mole %), preferably between 0.2% and 8% (mole %), more preferably between 0.3% and 7% (mole %) of the total fatty acid content of the plant, part thereof or plant cell.

In one embodiment, the plant, part thereof or plant cell has decreased levels of GLA. As used herein, “decreased levels of GLA” may be relative to a wild-type plant and/or a DHA1 construct. Preferably the GLA content is 5% (mole %) or less, preferably 4% (mole %) or less, more preferably 3% (mole %) or less, even more preferably 2.5% (mole %) or less, yet even more preferably 2.2% (mole %) or less, and most preferably 2.15% (mole %) or less of the total fatty acid content of the plant, part thereof or plant cell. In a preferred embodiment, GLA constitutes between 0.1% and 2.5% (mole %), preferably between 0.5% and 2.2% (mole %), more preferably between 1% and 2.15% (mole %) of the total fatty acid content present of the plant, part thereof or plant cell. In a preferred embodiment, GLA constitutes between 0.1% and 2.5% (mole %), preferably between 0.5% and 2.2% (mole %), more preferably between 1% and 2.15% (mole %) of the total fatty acid content of the plant, part thereof or plant cell. In another embodiment, the plant, part thereof or plant cell has decreased levels of erucic acid. Preferably the erucic acid content is 3.5% (mole %) or less, preferably 3% (mole %) or less, more preferably 2.5% (mole %) or less, even more preferably 2% (mole %) or less, yet even more preferably 1.5% (mole %) or less, and most preferably 1.2% (mole %) or less of the total fatty acid content of the plant, part thereof or plant cell. In a preferred embodiment, erucic acid constitutes between 0.1% and 2.5% (mole %), preferably between 0.5% and 2.0% (mole %), more preferably between 1% and 1.5% (mole %) of the total fatty acid content of the plant, part thereof or plant cell.

In one embodiment, the plant, part thereof or plant cell has an increased production of EPA and DHA. As used herein, an “increased production of EPA and DHA” may be relative to a wild-type plant and/or a DHA1 construct. Preferably the combined total of EPA and DHA is more than 13% (mole %), more preferably more than 15% (mole %), even more preferably more than 17% (mole %), yet even more preferably more than 20% (mole %), and yet even more preferably more than 25% (mole %) of the total fatty acid content of the plant, part thereof or plant cell. In a preferred embodiment, the combined total of EPA and DHA in the plant seed oil constitutes between 13% and 45% (mole %), more preferably between 15% and 40% (mole %), and even more preferably between 20% and 35% (mole %) of the total fatty acid content of the plant, part thereof or plant cell.

In another embodiment, the amount of total omega-3 fatty acids is increased. As used herein, an “increased amount of total omega-3 fatty acids” may be relative to a wild-type plant and/or a DHA1 construct. Preferably the amount of total omega-3 fatty acids is at least 30% (mole %) of the total fatty acid content present in said oil. Preferably, the amount of total omega-3 fatty acids is at least 40% (mole %), and more preferably at least 50% (mole %) of the total fatty acid content of the plant, part thereof or plant cell. In a preferred embodiment, the amount of total omega-3 fatty acids in the plant seed oil is between 30% and 60% (mole %), and more preferably between 40% and 60% (mole %) of the total fatty acid content of the plant, part thereof or plant cell.

In another embodiment, the amount of total omega-6 fatty acids is decreased. As used herein, a “decreased amount of total omega-6 fatty acids” may be relative to a wild-type plant and/or a DHA1 construct. Preferably the amount of total omega-6 fatty acids is less than 30% (mole %) of the total fatty acid content present in said oil, more preferably less than 25% (mole %), and even more preferably less than 22% (mole %) of the total fatty acid content of the plant, part thereof or plant cell. In a preferred embodiment, the amount of total omega-6 fatty acids in the plant seed oil is between 10% and 30% (mole %), and more preferably between 10% and 25% (mole %) of the total fatty acid content of the plant, part thereof or plant cell.

In a further embodiment, the amount of carotenoid content is increased. As used herein, an “increased amount of carotenoid content” may be relative to a wild-type plant and/or a DHA1 construct. Preferably the carotenoid content is at least 100 mg per kg, more preferably at least 150 mg per kg, even more preferably at least 200 mg per kg, yet even more preferably at least 300 mg per kg, yet even more preferably at least 400 mg per kg, and most preferably at least 500 mg per kg, of the plant, part thereof or plant cell. In a preferred embodiment, the carotenoid content is between 100 mg and 1000 mg per kg, preferably between 150 mg and 800 mg per kg, and even more preferably between 150 mg and 600 mg per kg, of the plant, part thereof or plant cell.

In another embodiment, the amount of ketocarotenoid content is increased. As used herein, an “increased amount of ketocarotenoid content” may be relative to a wild-type plant and/or a DHA1 construct. Preferably the ketocarotenoid content is at least 100 mg per kg, more preferably at least 150 mg per kg, even more preferably at least 200 mg per kg, yet even more preferably at least 300 mg per kg, yet even more preferably at least 400 mg per kg, and most preferably at least 500 mg per kg, of the plant, part thereof or plant cell.

In another embodiment, the amount of astaxanthin content is increased. As used herein, an “increased amount of astaxanthin content” may be relative to a wild-type plant and/or a DHA1 construct. Preferably the astaxanthin content is at least 20 mg per kg, more preferably at least 25 mg per kg, even more preferably at least 30 mg per kg, yet even more preferably at least 50 mg per kg, and yet even more preferably at least 100 mg per kg, of the plant, part thereof or plant cell. In a preferred embodiment, the astaxanthin content is between 20 mg per kg to 200 mg per kg, preferably between 25 mg per kg to 180 mg per kg, more preferably between 30 mg per kg to 170 mg per kg, even more preferably between 50 mg per kg to 160 mg per kg, yet even more preferably between 100 mg per kg to 150 mg per kg, of the plant, plant thereof or plant cell.

In another embodiment, the beta-carotene content, and in particular for where the plant, plant thereof or plant cell comprises astaxanthin, is less than 1 mg per kg, preferably less than 0.5 mg per kg, more preferably less than 0.2 mg per kg, even more preferably less than 0.1 mg per kg, yet even more preferably less than 0.05 mg per kg, most preferably less than 0.03 mg per kg, of the plant, part thereof or plant cell.

As used herein, the term “astaxanthin” includes all stereoisomers and tautomers of astaxanthin, including all geometric isomers, enantiomers and diastereomers of astaxanthin.

In another embodiment, the ratio of total C20+n-3/C20+n-6 fatty acids and/or the ratio of omega-3/omega-6 LC-PUFAs is increased. As used herein, an “increased ratio of total C20+n-3/C20+n-6 fatty acids” and/or an “increased ratio of omega-3/omega-6 LC-PUFAs” may be relative to a wild-type plant and/or a DHA1 construct.

In another aspect of the invention, there is provided the use of the recombinant plant of the present invention to produce or increase production of omega-3 LC-PUFAs and/or to increase the ratio of omega-3 to omega-6 fatty PUFAs.

In a further aspect of the invention there is provided a method of producing the recombinant plant of the present invention, the method comprising introducing and expressing a nucleic acid construct comprising at least one nucleic acid sequence encoding at least a Δ6-elongase, a Δ5-desaturase and a Δ6-desaturase before, after or concurrently with reducing or abolishing the expression at least one FAE1 gene by any of the above-described methods. For example, the method may comprise introducing and expressing one of the DHA or EPA constructs of the invention before, after or concurrently with a genome editing (e.g. sgRNA construct targeting FAE1), again as described above.

In another aspect of the invention, there is provided a method of producing the recombinant plant of the present invention, the method comprising introducing and expressing a nucleic acid construct comprising at least one nucleic acid sequence encoding at least a Δ6-elongase, a Δ5-desaturase and a Δ6-desaturase in a first plant, reducing or abolishing the expression of at least one FAE1 gene in a second plant and crossing the first and second plant, wherein the progeny express the nucleic acid construct and have reduced or abolished expression of FAE1. Alternatively, the method may comprise transference of any of the nucleic acid constructs of the invention by crossing, e.g., using pollen of the genetically altered plant that expresses one of the above EPA or DHA constructs to pollinate a plant containing one or more mutation in at least one FAE1 gene.

In another aspect of the invention, there is provided a method of producing a recombinant plant, part thereof or plant cell with increased omega-3 LC-PUFAs content, the method comprising cultivating the recombinant plant, part thereof or cell under conditions which allow the production of one or more omega-3 LC-PUFAs, and obtaining said omega-3 LC-PUFAs from the plant, part thereof or cell. In one embodiment, the omega-3 LC-PUFAs is selected from at least one of EPA, DPA and DHA.

In another aspect of the invention, there is provided a method of producing a recombinant plant, part thereof or plant cell, the method comprising introducing and expressing a nucleic acid construct comprising nucleic acid sequences encoding a Δ6-elongase, a Δ5-desaturase, Δ6-desaturase, a hydroxy-beta-ring 4-dehydrogenase (HBFD) and a Keto2, and optionally a phytoene synthase, wherein preferably the nucleic acid sequences are operably linked to at least one regulator sequence.

In another aspect of the invention, there is provided a method of producing a recombinant plant, part thereof or plant cell, the method comprising introducing and expressing a nucleic acid construct comprising nucleic acid sequences encoding a Δ6-elongase, a Δ5-desaturase, Δ6-desaturase, a hydroxy-beta-ring 4-dehydrogenase (HBFD) and a Keto2, and optionally a phytoene synthase, wherein preferably the nucleic acid sequences are operably linked to at least one regulator sequence

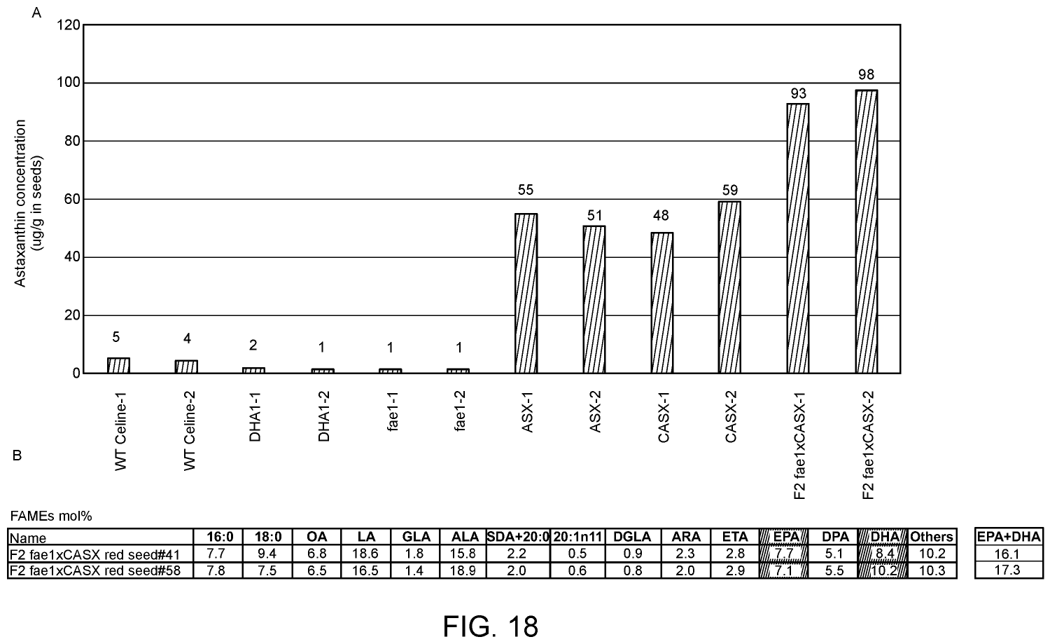

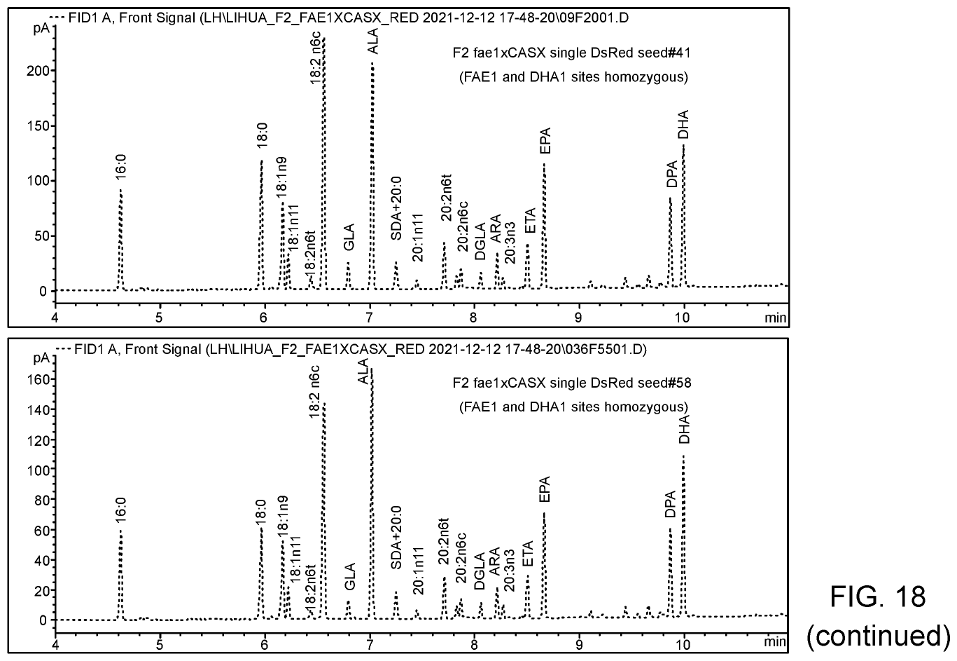

In another aspect of the invention, there is provided a method of producing the recombinant plant, the method comprising introducing and expressing a first nucleic acid construct comprising at least one nucleic acid sequence encoding at least a Δ6-elongase, a Δ5-desaturase and a Δ6-desaturase, preferably linked to regulatory sequence (such as the DHA1 or EPA8 construct described herein) in a first plant and introducing and expressing a second construct into the same plant or a second plant, where the second nucleic acid construct comprises nucleic acid sequences encoding a hydroxy-beta-ring 4-dehydrogenase (HBFD) and a Keto2, and optionally a phytoene synthase, preferably linked to at least one regulatory sequence, and crossing the first and second plant, wherein the progeny express both the first and second constructs. These plants may be referred to as CASX lines herein. The method may further comprise the step of crossing the progeny with a third plant, where the third plant has reduced or abolished expression of at least one FAE1 gene. Alternatively the method may comprise transference of any of the nucleic acid constructs of the invention by crossing, e.g., using pollen of the recombinant plant that expresses one of the above EPA or DHA constructs (e.g. DHA1 or EPA8 construct described herein) to pollinate a plant containing a construct expressing at least one HBFD and/or Keto2 nucleic acid and/or one or more mutation in at least one FAE1 gene. FIGS. 18A and B shows that the plants from these crosses have significantly increased levels of astaxanthin (FIG. 18A) and EPA and DHA (FIG. 18B).

In another aspect of the invention, there is provided a method of modifying the TAG composition of plant seed oil, the method comprising growing a plant according to the invention under conditions wherein said desaturase and elongase enzymes are expressed.

In one embodiment, modifying the TAG composition comprises increasing the amount of triacylglycerol (TAG) species of 56 carbons and above in a plant, preferably plant seed oil, the method comprising growing a plant according to the invention under conditions wherein said desaturase and elongase enzymes are expressed. Preferably, the method comprises increasing the amount of TAG species between 58 to 66 carbons.

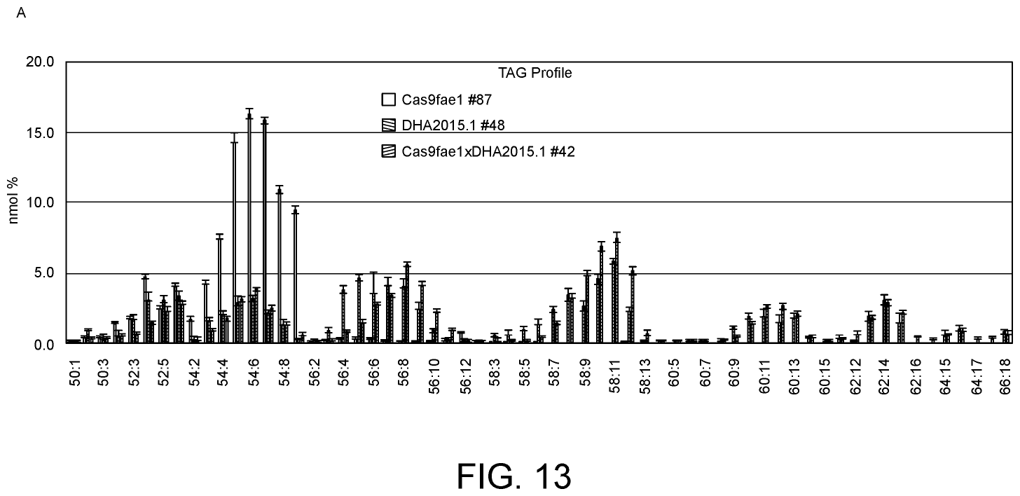

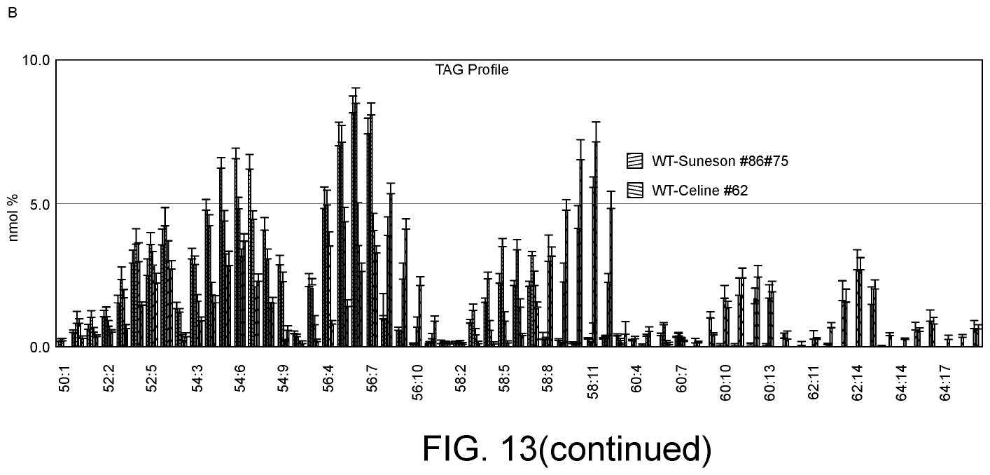

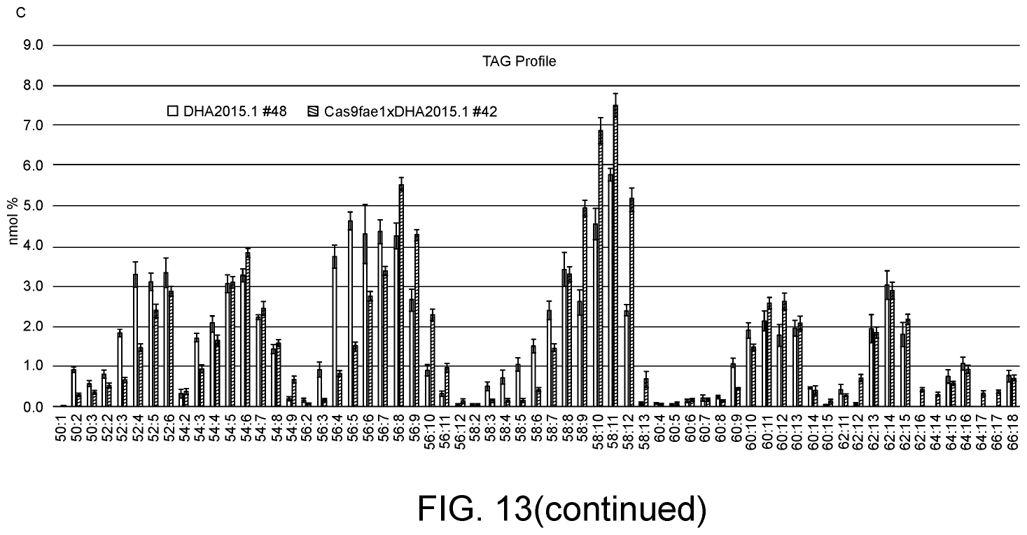

In another embodiment, modifying the TAG composition comprises increasing the amount of one or more of 54:9, 56:8, 56:9, 56:10, 56:11, 58:9, 58:10, 58:11, 58:12, 58:13, 60:12, 62:12, 62:16, 64:14, 64:17 and 66:17 TAG species. The increase in 58:8 to 58:12 TAG species is indicative of an increase in the production of LC-PUFAs. Preferably, the method comprises increasing the amount of 62:16, 64:14, 64:17 and 66:17 TAG species—in particular. As shown in FIG. 13 these species are not produced when DHA2015.1 is expressed alone, and as such, an increase in this context is equivalent to a method of producing one or more of the following TAG species 62:16, 64:14, 64:17 and 66:17. 66:17 TAG is particularly useful as this TAG is made up of the LC-PUFAs, DHA, DHA and DPA. As such, in one embodiment, there is provided a method of producing 66.17 TAG. Similarly, 60:12 TAG is also useful as it is made up of two LC-PUFAs, DHA and EPA as well as oleic acid. Accordingly, in another embodiment, there is provided a method of producing 60:12 TAG.

In another embodiment, modifying the TAG composition comprises increasing the amount of one or more of EPA, DHA and DPA in C58 or more (preferably between 58 and 66) TAGs species. In one embodiment, the method comprises increasing the EPA content of one or more of the following TAG species: 56:6, 56:7, 56:8, 56:9, 56:11, 58:10, 58:11, 58:12, 58:13, 60:10, 60:11, 60:12, 60:14, 62:14, 62:15, 62:16 and 64:17 and/or increasing the DPA content of one or more of the following TAG species: 58:7, 58:9, 58:11, 58:12, 60:9, 60:10, 60:13, 62:11, 62:13, 62:14, 62:15, 64:14, 64:15, 64:16, 66:16 and 66:17 and/or increasing the DPA content of one or more of the following TAG species: 56:9, 56:10, 58:13, 60:12, 60:13, 60:15, 62:7, 62:11, 62:12, 62:16, 64:14, 64:17, 66:16 and 66:17.

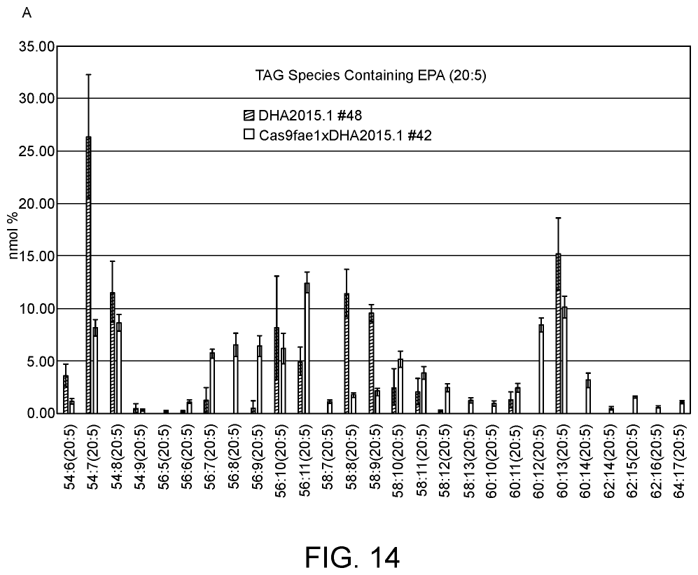

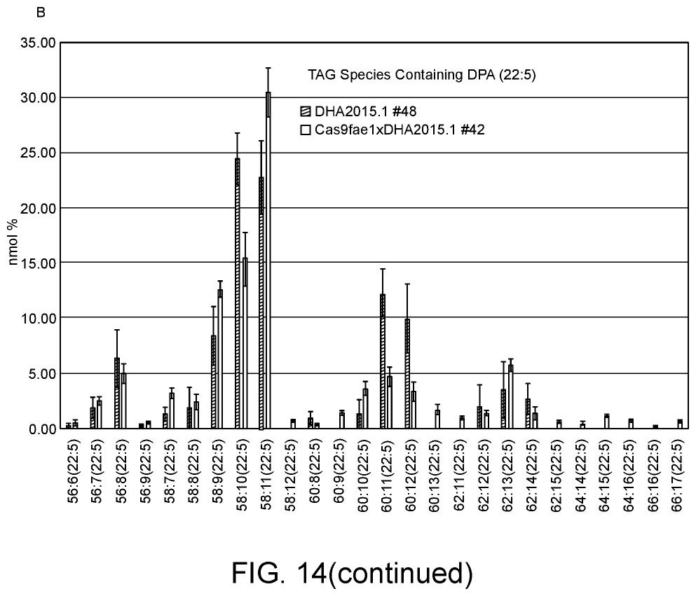

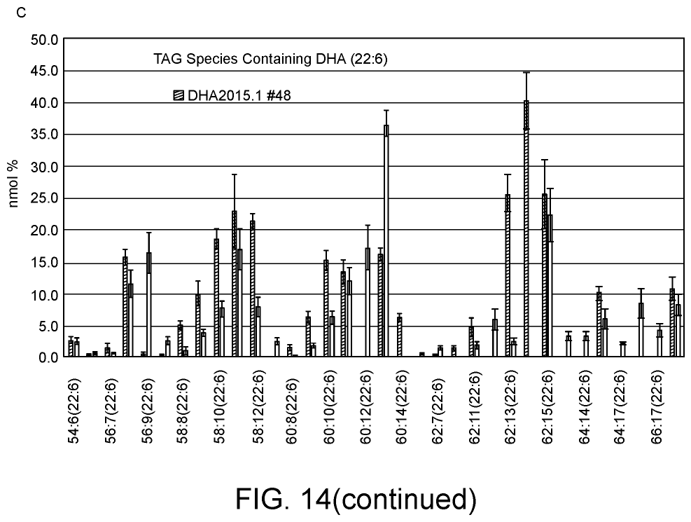

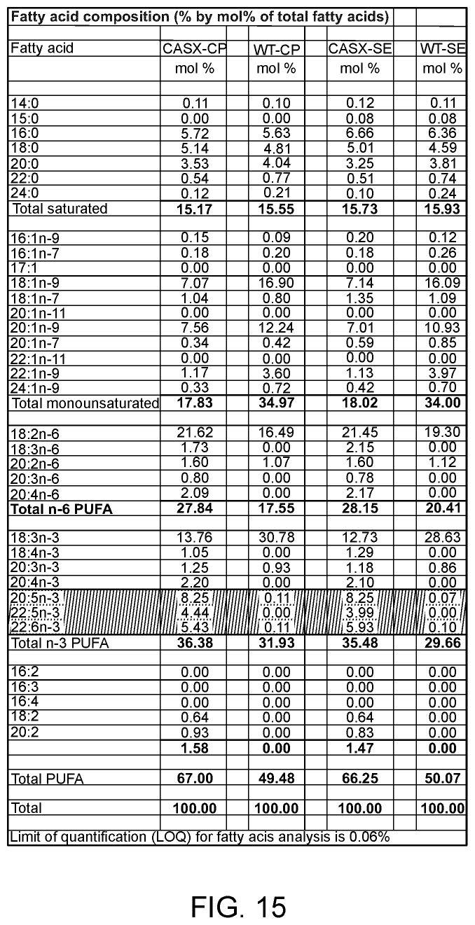

In a further embodiment, as shown in FIGS. 13 and 14, modifying the TAG composition comprises producing EPA-TAG and/or DPA-TAG and/or DHA-TAG compositions that are only present in plants of the invention (e.g. not in wild-type plants, plants of the fae1 background or plants expressing only the DHA2015.1 construct). In one embodiment, the EPA-TAG is one or more of the following TAG species: 56:8, 58:7, 58:12, 58:13, 60:10, 60:12, 60:14, 62:14, 62:15, 62:16 and 64:17; the DPA-TAG is one or more of the following TAG species: 58:12, 60:9, 60:13, 62:11, 62:15, 64:14, 64:15, 64:16, 66:16 and 66:17; the DHA-TAG is one or more of the following TAG species: 58:13, 60:12, 62:12, 62:16, 64:14, 64:17, 66:16 and 66:17.

In another embodiment, there is provided a method of increasing the diversity of TAG species in a plant, preferably plant seed oil, the method comprising growing a plant according to the invention under conditions wherein said desaturase and elongase enzymes are expressed. By increasing diversity is meant increasing the repertoire of TAG species produced. For example, as described in FIGS. 13 and 14, plants of the invention produce one or more of the following TAG species: 54:9, 56:8, 56:9, 56:10, 56:11, 58:9, 58:10, 58:11, 58:12, 58:13, 60:12, 62:12, 62:16, 64:14, 64:17, 66:16 and 66:17 TAG species that are not produced in the fae1 background alone or when DHA2015.1 is expressed in the wild-type background.

The method may further comprise selecting one or more mutated plant cells or plants, preferably for further propagation. The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In another aspect of the invention, there is provided a method for producing plant seed oil, comprising growing a plant, part thereof or cell of the present invention under conditions wherein said desaturase and elongase enzymes are expressed and a plant seed oil is produced in said plant, part thereof or cell.

In one embodiment, the plant seed oil is produced by cold pressing.

In another embodiment, the plant seed oil is produced by solvent extraction. The solvent used in a solvent extraction process is not particularly limited and may include solvents selected from hydrocarbons, such as pentane, hexane, petroleum ether; alcohols, such as methanol, ethanol, isopropanol and butanol; ethers, such as diethyl ether and THF; acetone; chlorinated solvents, such as dichloromethane and chloroform; and supercritical CO₂.

In a further aspect there is provided a plant seed oil produced by the method of the present invention.

In one embodiment, the oil is a cold-pressed oil.

In another embodiment, the oil is a solvent-extracted oil.

In further embodiments, the oil may be a blend of a cold-pressed oil and a solvent-extracted oil.

In another aspect of the invention, there is provided a plant seed oil comprising EPA and/or DHA, wherein EPA constitutes at least 5% (mole %) of the total fatty acid content present in said oil and wherein DHA constitutes at least 5% (mole %) of the total fatty acid content present in said oil; and wherein the oil has a gondoic acid (GA) content of 10% (mole %) or less based on the total fatty acid content present in said oil, and/or wherein the oil has a ketocarotenoid content of at least 100 mg per kg of said oil.

In one embodiment, the plant seed oil comprises DHA. In one embodiment, DHA constitutes at least 5% (mole %), preferably 6% (mole %) or more, more preferably 7% (mole %) or more, even more preferably 10% (mole %) or more, yet even more preferably 12% (mole %), and most preferably 15% (mole %) or more of the total fatty acid content present in said oil. In a preferred embodiment, DHA constitutes between 5% and 30% (mole %), preferably between 5% and 25% (mole %), more preferably between 5% and 20% (mole %), and even more preferably between 10% and 20% (mole %) of the total fatty acid content present in said oil.

In a further embodiment, the plant seed oil comprises EPA. In one embodiment, EPA constitutes at least 5% (mole %), preferably 7% (mole %) or more, more preferably 8% (mole %) or more, even more preferably 9% (mole %) or more, and yet even more preferably 10% (mole %) or more of the total fatty acid content present in said oil. In a preferred embodiment, EPA constitutes between 8% and 30% (mole %), preferably between 8% and 25% (mole %), and more preferably between 8% and 20% (mole %) of the total fatty acid content present in said oil.

In a further embodiment, the plant seed oil comprises DPA. In one embodiment, DPA constitutes at least 3% (mole %), preferably 4% (mole %) or more, more preferably 5% (mole %) or more, and even more preferably 6% (mole %) or more of the total fatty acid content present in said oil. In a preferred embodiment, DPA constitutes between 3% and 30% (mole %), preferably between 3% and 25% (mole %), and more preferably between 3% and 20% (mole %) of the total fatty acid content present in said oil.

In another embodiment, the plant seed oil has a GA content of 10% (mole %) or less, preferably 8% (mole %) or less, more preferably 7% (mole %) or less, even more preferably 6% (mole %) or less, yet even more preferably 5% (mole %) or less, most preferably 4% (mole %) or less of the total fatty acid content present in said oil. In a preferred embodiment, GA constitutes between 0.1% and 9% (mole %), preferably between 0.2% and 8% (mole %), more preferably between 0.3% and 7% (mole %) of the total fatty acid content present in said oil.

In another embodiment, the plant seed oil has a GLA content of 5% (mole %) or less, preferably 4% (mole %) or less, more preferably 3% (mole %) or less, even more preferably 2.5% (mole %) or less, yet even more preferably 2.2% (mole %) or less, and most preferably 2.15% (mole %) or less of the total fatty acid content present in said oil.

In a preferred embodiment, GLA constitutes between 0.1% and 2.5% (mole %), preferably between 0.5% and 2.2% (mole %), more preferably between 1% and 2.15% (mole %) of the total fatty acid content present in said oil.

In another embodiment, the plant seed oil has an erucic acid content of 3.5% (mole %) or less, preferably 3% (mole %) or less, more preferably 2.5% (mole %) or less, even more preferably 2% (mole %) or less, yet even more preferably 1.5% (mole %) or less, and most preferably 1.2% (mole %) or less of the total fatty acid content present in said oil. In a preferred embodiment, erucic acid constitutes between 0.1% and 2.5% (mole %), preferably between 0.5% and 2.0% (mole %), more preferably between 1% and 1.5% (mole %) of the total fatty acid content present in said oil.

In a further embodiment, the combined total of EPA and DHA in the plant seed oil constitutes more than 13% (mole %), preferably more than 15% (mole %), more preferably more than 17% (mole %), even more preferably more than 20% (mole %), and yet even more preferably more than 25% (mole %) of the total fatty acid content present in said oil. In a preferred embodiment, the combined total of EPA and DHA in the plant seed oil constitutes between 13% and 45% (mole %), more preferably between 15% and 40% (mole %), and even more preferably between 20% and 35% (mole %) of the total fatty acid content present in said oil.

In a further embodiment, the amount of total omega-3 fatty acids in the plant seed oil is at least 30% (mole %) of the total fatty acid content present in said oil. Preferably, the amount of total omega-3 fatty acids is at least 40% (mole %), and more preferably at least 50% (mole %) of the total fatty acid content present in said oil. In a preferred embodiment, the amount of total omega-3 fatty acids in the plant seed oil is between 30% and 60% (mole %), and more preferably between 40% and 60% (mole %) of the total fatty acid content present in said oil.

In a further embodiment, the amount of total omega-6 fatty acids in the plant seed oil is less than 30% (mole %) of the total fatty acid content present in said oil. Preferably, the amount of total omega-6 fatty acids is less than 25% (mole %), and more preferably less than 22% (mole %) of the total fatty acid content present in said oil. In a preferred embodiment, the amount of total omega-6 fatty acids in the plant seed oil is between 10% and 30% (mole %), and more preferably between 10% and 25% (mole %) of the total fatty acid content present in said oil.

In a further embodiment, the plant seed oil comprises a carotenoid. In one embodiment, the oil has a carotenoid content of at least 100 mg per kg of the oil. Preferably, the carotenoid content is at least 150 mg per kg of the oil, more preferably at least 200 mg per kg of the oil, even more preferably at least 300 mg per kg of the oil, yet even more preferably at least 400 mg per kg of the oil, and most preferably at least 500 mg per kg of the oil. In a preferred embodiment, the carotenoid content is between 100 mg and 1000 mg per kg of the oil, preferably between 150 mg and 800 mg per kg of the oil, and even more preferably between 150 mg and 600 mg per kg of the oil.

In a further embodiment, the plant seed oil comprises a ketocarotenoid. In one embodiment, the oil has a ketocarotenoid content of at least 100 mg per kg of the oil. Preferably, the ketocarotenoid content is at least 150 mg per kg of the oil, more preferably at least 200 mg per kg of the oil, even more preferably at least 250 mg per kg of the oil, yet even more preferably at least 300 mg per kg of the oil, and most preferably at least 400 mg per kg of the oil. In a preferred embodiment, the ketocarotenoid content is between 100 mg and 1000 mg per kg of the oil, preferably between 150 mg and 800 mg per kg of the oil, and even more preferably between 150 mg and 600 mg per kg of the oil.

In a further embodiment, the ketocarotenoid forms more than 50%, preferably more than 60%, more preferably more than 70%, even more preferably more than 75%, and yet even more preferably more than 80% of the total carotenoid content. In a preferred embodiment, the ketocarotenoid forms between 50% to 99%, preferably between 60% to 98%, more preferably between 70% to 97%, even more preferably between 75% to 95%, and yet even more preferably between 80% to 90% of the total carotenoid content.

In a further embodiment, the plant seed oil comprises astaxanthin. In one embodiment, the astaxanthin content is at least 20 mg per kg of the oil, preferably at least 25 mg per kg of the oil, more preferably at least 30 mg per kg of the oil, even more preferably at least 50 mg per kg of the oil, yet even more preferably at least 100 mg per kg of the oil.

In a preferred embodiment, the astaxanthin content is between 20 mg per kg to 200 mg per kg of the oil, preferably between 25 mg per kg to 180 mg per kg of the oil, more preferably between 30 mg per kg to 170 mg per kg of the oil, even more preferably between 50 mg per kg to 160 mg per kg of the oil, yet even more preferably between 100 mg per kg to 150 mg per kg of the oil. The astaxanthin may be present as various stereoisomers (e.g. geometric, diastereomeric or enantiomeric isomers) and/or may optionally be present in an esterified form. Preferably, the astaxanthin is present in a free (i.e. non-esterified) form.

In a further embodiment, the plant seed oil, and in particular for where the plant seed oil comprises astaxanthin, has a beta-carotene content of less than 1 mg per kg of the oil, preferably less than 0.5 mg per kg of the oil, more preferably less than 0.2 mg per kg of the oil, even more preferably less than 0.1 mg per kg of the oil, yet even more preferably less than 0.05 mg per kg of the oil, most preferably less than 0.03 mg per kg of the oil.

Accordingly, in a further aspect of the invention there is provided a method of increasing the ketocarotenoid content, preferably astaxanthin content of plant seed oil, the method comprising expressing the nucleic acid sequences described herein in a plant. In a further aspect of the invention, there is provided a method of increasing the ketocarotenoid content, preferably astaxanthin content and the omega-3 LC-PUFAs content of plant seed oil, the method comprising expressing the nucleic acid sequences described herein in a plant.

Preferably the plant part thereof is a seed. Accordingly, in a further aspect of the invention, there is provided a seed obtained or obtainable by the plant of the present invention. There is also provided progeny obtained or obtainable from the plant seed of the invention as well as plant seed obtained or obtainable from the progeny. In another aspect of the invention there is provided a feedstuff, food, cosmetic or pharmaceutical comprising the oil of the present invention. Preferably the feedstuff is an aquafeed for use in aquaculture.

The terms “introduction”, “transfection” or “transformation” as referred to herein encompass the transfer of an exogenous polynucleotide into a host cell, irrespective of the method used for transfer. Such terms may be used to refer to the introduction of the nucleic acid constructs of the invention or the CRISPR constructs described herein into a host cell. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

The transfer of foreign genes into the genome of a plant is called transformation. Transformation of plants is now a routine technique in many species. Any of several transformation methods known to the skilled person may be used to introduce the nucleic acid construct of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant (microinjection), gene guns (or biolistic particle delivery systems (biolistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibres, electroporation of protoplasts, microinjection into plant material, DNA or RNA-coated particle bombardment, infection with (non-integrative) viruses and the like.

Recombinant plants can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct molecule or CRIPSR construct as described herein can be introduced to at least one plant cell using any of the above described methods.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be DsRed. Alternatively, the transformed plants are screened for the presence of a selectable marker, such as, but not limited to, GFP, GUS (β-glucuronidase). Other examples would be readily known to the skilled person. Alternatively, no selection is performed, and the seeds obtained in the above-described manner are planted and grown and omega-3 LCPUFA measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, both techniques being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

Transformation systems for camelina are also known in the art. For example, the following protocol can be followed:
1. Grow healthy Camelina plants at 21° C. day/18° C. night at long day condition. Each construct for 12 pots (11 cm diameter), 4 plants/pot.
2. Wait until plants to start flowering, at 5-6 weeks' time from sowing.
3. Prepare Agrobacterium tumefaciens strain carrying gene of interest on a binary vector.
4. Grow a pre-culture of Agrobacterium of 5 ml in LB with antibiotics. Overnight at 28° C.
5. Inoculate a large liquid culture (400 ml) with the 5 ml pre-culture and grow it at 28° C. overnight, in LB with antibiotics to select for the binary plasmid.
6. Spin down Agrobacterium (4500 rpm 15 min), resuspend in 250 ml of infiltration media (made fresh, no need to autoclave): 5% Sucrose solution and 0.05% Silwet L-77 (500 μl/L)+½ MS (Murashige and Skoog nutrition).
7. Dip flower parts of plant in Agrobacterium solution for 1 minute with gentle agitation.
8. Label the pot with Construct name, Date and Researcher name.
9. Bag the dipped flower parts for 24 hours to maintain high humidity. Further cover the whole plants with black plastic bag to keep darkness.
10. Open the bags, and tie plants in the pot with a stick as long as plant high. Water and grow plants normally.
11. Stop watering as seeds become mature. Do not water on the top part (seeds and flowers).
12. Harvest dry seeds.
13. Select for transformants using fluorescence, antibiotics or herbicide (BASTA) selectable marker.

Recombinant plants which comprise the polyunsaturated fatty acids synthesized in the process according to the invention can advantageously be marketed directly without there being any need for the oils, lipids or fatty acids synthesized to be isolated.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein or carry the herein described mutations. The term “plant” also encompasses plant cells, suspension cultures, callus tissue, embryos, meristematic regions, gametophytes, sporophytes, pollen and microspores, again wherein each of the aforementioned comprises the nucleic acid construct or mutations as described herein. In one example only the plant cell is a cell that is not capable of photosynthesis. For example, the plant cell may lack chloroplasts. The cell may also be from one of the following tissue types, including leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. The invention also relates to food products and food supplements comprising the plant of the invention or parts thereof. In one embodiment, the food products may be animal feed. In another aspect of the invention, there is provided a product derived from a plant as described herein or from a part thereof. In a most preferred embodiment, the plant part or harvestable product is a seed or grain. Therefore, in a further aspect of the invention, there is provided a seed produced from a genetically altered plant as described herein.

The compounds produced in the process according to the invention can also be isolated from the organisms, advantageously plants, in the form of their oils, fats, lipids and/or free fatty acids. This can be done via pressing or extraction of the camelina plant parts, preferably the plant seeds. In this context, the oils, fats, lipids and/or free fatty acids can be obtained by what is known as cold-beating or cold-pressing without applying heat. To allow for greater ease of disruption of the plant parts, specifically the seeds, they are previously comminuted, steamed or roasted. The seeds which have been pretreated in this manner can subsequently be pressed or extracted with solvents such as warm hexane. Thereafter, the resulting products are processed further, i.e. refined. In this process, substances such as the plant mucilages and suspended matter are first removed. What is known as desliming can be effected enzymatically or, for example, chemico-physically by addition of acid such as phosphoric acid. Thereafter, the free fatty acids are removed by treatment with a base, for example sodium hydroxide solution. The resulting product is washed thoroughly with water to remove the alkali remaining in the product and then dried. To remove the pigment remaining in the product, the products are subjected to bleaching, for example using filler's earth or active charcoal. At the end, the product is deodorized, for example using steam.

In the case of plant (including plant tissue or plant organs) or plant cells, “growing” is understood as meaning, for example, the cultivation on or in a nutrient medium, or of the intact plant on or in a substrate, for example in a hydroponic culture, potting compost or on arable land.

In one embodiment, the plant is an oilseed plant. In a preferred embodiment, the plant selected from the family Brassicaceae. In one embodiment, the plant is Camelina. Camelina is a superior boutique platform for the production of these important oils (Napier et al., 2018; Tocher et al., 2019). For both species (camelina, canola), the accumulation of EPA and DHA was achieved by the seed-specific expression of the non-native omega-3 LC-PUFA biosynthetic pathway, a suite of genes predominantly derived from marine microorganism such as phytoplankton, which in the most minimal form requires the presence of five distinct and sequential enzyme activities to convert the C18 fatty acids ubiquitous to higher plants into the non-native C20+ PUFA forms Petrie et al., 2014; Ruiz-Lopez et al 2014). Progress has been made in demonstrating the successful metabolic engineering of this pathway in Arabidopsis and camelina, though this has inevitably been based on an iterative approach to construct design (Usher et al., 2017).

The Camelina plant may be selected from C. alyssum, C. microcarpa, C. rumelica and C. sativa. Most preferably, the Camelina is C. sativa. In another embodiment, the plant is selected from B. napus, B. rapa, B. juncea, B. carinata and B. hirta.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The invention is now described in the following non-limiting examples.

### Example I: Design of the DHA and EPA Constructs

### Design of DHA2015.1

DHA201.1 contains a Δ6-desaturase gene from O. tauri (OtA6), a Δ6 fatty acid elongase gene from Physcomitrella patens (PSE1), a Δ5-desaturase gene from Thraustochytrium sp. (TcΔ5), a Δ12-desaturase gene from Phytophthora sojae (PsΔ12), an ω3-desaturase from Phytophthora infestans (Piw3) and an O. tauri Δ5 fatty acid elongase gene (OtElo5) and a Δ4-desaturase gene from Ostreococcus RCC809 (0809D4). All open reading frames for desaturases and elongases were re-synthesized (GenScript Corporation, NJ, www.genscript.com) and codon optimized for expression in Brassica. All genes were individually cloned under the control of seed-specific promoters and then combined into a single T-DNA transformation vector as previously described (Ruiz Lopez et al., 2014). The destination binary vector contained a DsRed marker within the T-DNA sequence for visual selection of GM plants.

### Design of Optimised Constructs

As used herein, the “optimised constructs” refer to at least DHA2015.5, EPA2015.8 and EPA2016.1.

Further using the DHA2015.1 (Han et al., 2020) as the prototype for systematic improvement, we then built a number of variant forms in which individual changes to genetic elements are accumulated [FIG. 4]. DHA2015.1 comprises seven genes encoding activities on the biosynthetic pathway for EPA and DHA, including the five primary enzymes (Δ6-, Δ5- and Δ4-desaturases; Δ6- and Δ5-elongases) and two additional activities (Δ12-desaturase, ω3-desaturase) proposed to be involved in maximising substrate fluxes (Napier et al., 2015; FIGS. 1 & 2). We have found that the Phytophora sojae Δ12-desaturase (PsD12) might be dispensable, since this activity not only depleted the oleic acid (18:1n-9) pool but also generated excessive omega-6 fatty acids. Therefore, this activity was omitted from the next three derivations (named DHA2015.2-4)—thus these 3 constructs contained one less expression cassette than DHA2015.1 (FIG. 2). In addition, DHA2015.2 replaced the Phytophora infestans ω3-desaturase (Piw3) with a similar activity from Hyaloperonospora parasitica (Hpw3), DHA2015.3 replacing the Ostreococcus tauri ELO5 activity with a similar one from Ostreococcus RCC809 (0809Elo5) along with the Hpw3, and DHA2015.4 replacing Ostreococcus RCC809 Δ4-desaturase with a similar activity from Thalassiosira pseudonana, along with Hpw3 and 0809Elo5 (FIG. 4).

By this this systematic approach we could better define critical steps in the pathway and optimal combinations of transgene-derived activities. In addition, we also made a further variant of DHA2015.1 in which all the original activities were retained, but an additional activity (Perilla frutescens FAD3/Δ15-desaturase -PerfD15) was added [FIG. 1—this construct was named DHA2015.5 [FIG. 4]. A very similar approach was adopted with the systematic improvement of construct EPA_B4.1 [FIG. 4] which was previously shown to accumulate high levels of EPA (Usher et al., 2017). In the new iteration named EPA2016.1 the O. tauri Δ6-desaturase was replaced with a similar activity from Mantoniella squamata (MsqD6), in EPA2015.4, the Δ5-desaturase from Thraustochytrium spp. was replaced with a similar activity from Emiliania huxleyi, in addition to the replacement of the O. tauri Δ6 and in EPA2015.8, the prototype was amended by the addition of the PerfD15 Δ15-desaturase activity, analogous to DHA2015.5. All of these constructs were assembled as previously described and introduced in Camelina via Agrobacterium-mediated transformation (Ruiz-Lopez et al., 2014).

### Example II: Materials and Methods

### Plant Material and Growth Conditions

Camelina sativa (cv. Celine) was used in all experiments. Plants grown in the glasshouse were maintained in controlled conditions at 23° C. day/18° C. night, 50-60% humidity and kept under a 16-h photoperiod (long day), with supplemental light provided when ambient levels fell below 400 μmol/m2/s. Harvest usually occurred 100 days after sowing.

### Generation of Recombinant Plants

Recombinant C. sativa lines were generated as previously described (Ruiz-Lopez et al., 2014). The designed vectors were transferred into Agrobacterium tumefaciens strain AGL1. C. sativa inflorescences were immersed in the Agrobacterium suspension for 1 min without applying any vacuum. Recombinant seeds expressing the EPA and DHA pathway were identified by visual screening for DsRed activity. Seeds harvested from transformed plants were illuminated using a green LED light. Fluorescent seeds were visualized using a red lens filter.

### Vector Construction

All genes described above were individually cloned under the control of seed-specific promoters and then combined into a single T-DNA transformation vector as previously described (Ruiz Lopez et al., 2014). The destination binary vector contained a DsRed marker within the T-DNA sequence for visual selection of GM plants.

### Field Trials

Field experiments conducted at Rothamsted Research in 2016 and 2017 (Harpenden, Hertfordshire, U.K.; grid reference TL120130) were carried out as previously described (Usher et al., 2015, 2017), under DEFRA consent 16/R8/01. Field trials in Canada were managed by Ag-Quest (Minto, Manitoba; https://agquest.com) including all aspects of approvals from CFIA for environmental release. Similarly, field trials in USA were managed by University of Nebraska, Lincoln experimental farm facility, part of the Department of Agriculture and Horticulture, including obtaining approvals from APHIS for environmental release. Unless stated otherwise, for all the experimental data analysis, the values of each Camelina line were given as mean value ±standard error from each line replicate plots.

### Assessment of Aqronomic Performance

Total seed oil was measured by NMR. Each seed sample (about 2 g) is placed into the NMR tube, weighted and measured and then calculated the oil content according to the calibration curve. Thousand grain weight is measured by weighing 1000 dry seeds. For seed oil and TGW analysis, one sample is collected from each plot. Technical replicates were then drawn from this single sample.

### Fatty Acid Analysis

Total fatty acids in seed batches were extracted and transmethylated according to previous methods (Ruiz-Lopez et al., 2014). Four biological replicates were sampled from each plot, with the amount of 100 mg dry seeds each replicate. Methyl ester derivatives of total fatty acids extracted were analysed by Gas Chromatography-FID (flame ionization detection), and the results were confirmed by GC-MS. Minor fatty acids (such as 16:1n-7, 18:2trans, 20:1n-7, 20:2trans, 22:0, 22:2n-6 and 24:0) were summed and are presented as others.

### Lipid Analysis

Triacylglycerols (TAGs) were measured in Camelina seed from seed harvested from the field trial. The sampling method is the same with that of fatty acid analysis. TAGs were measured according to Usher et al. (2017) and were defined by the presence of one acyl fragment and the mass/charge of the ion formed from the intact lipid (neutral loss profiling). This allows identification of one TAG acyl species and the total acyl carbons and total number of acyl double bonds in the other two chains. The procedure does not allow identification of the other two fatty acids individually nor the positions (sn-1, sn-2, or sn-3) that individual acyl chains occupy on the glycerol. TAGs were quantified after background subtraction, smoothing, integration, isotope deconvolution and comparison of sample peaks with those of the internal standard (using Lipid-View™; Sciex). The data were normalized to the internal standards tri15:0 and tri19:0 (Nu-Chek Prep, Elysian, MN). The profiling samples were prepared by combing 50 uL of the total lipid extract with 950 uL of isopropanol/methanol/50 mm ammonium acetate/dichloromethane (4:3:2:1). Samples were infused at 15 uL/min with an autosampler (CTC-PAL, CTC Analytics). The scan speed was 100 u/s. The collision energy, with nitrogen in the collision cell, was +25 V; declustering potential was +100 V; entrance potential was 14 V; and exit potential was +14 V. Sixty continuum scans were averaged in the multiple channel analyser mode. For product ion analysis, the first quadrupole mass spectrometer (Q1) was set to select the TAG mass and Q3 for the detection of fragments fragmented by collision induced dissociation. The mass spectral responses of various TAG species are variable, owing to differential ionization of individual molecular TAG species. For all analyses, gas pressure was set on ‘low’, and the mass analysers were adjusted to a resolution of 0.7 L full width height. The source temperature was 100° C.; the interface heater was on, and +5.5 kV was applied to the electrospray capillary; the curtain gas was set at 20 (arbitrary units); and the two ion source gases were set at 45 (arbitrary units). In the data shown herein, no response corrections were applied to the data. The data were normalized to the internal standards tri15:0 and tri19:0 (Nu-Chek Prep, Elysian, MN).

### Example III: Generation and Characterisation of DHA1×FAE1 Plants

The fae1 mutant (lacking the CRISPR-Cas9 transgene and associated DsRed marker) produced by Ozseyhan et al. (2018) which is incorporated herein by reference, was crossed with the DHA2015.1 line described in Han et al (2020), which is incorporated herein by reference, and the resulting F1 hybrid seeds were sown in the greenhouse. Resulting seeds from individual F2 plants were harvested and those with strong DsRed fluorescence were selected on the basis that these represented homozygosity at the DHA1 locus (since the T-DNA insertion for that trait contained the DsRed marker—FIG. 1b). Selected seeds were dissected, and half the cotyledon tissue was used for fatty acid composition analysis by GC-FID, the leftover seeds with half cotyledon and radicle were germinated on 1/2MS nutrition media. Based on the fatty acid methyl esters (FAMEs) data, only seeds showing a low 20:1Δ¹¹ content (less than 0.5%, as described in Ozseyhan et al., 2018) were identified as the DHA1 and fae1 homozygous line, then they were bulked up in the greenhouse. The harvested F3 seeds, with parallel parental lines and WTs, were sown at the Rothamsted Experimental Farm, Harpenden, UK on 23 May 2019, with permission from 19/R8/01 DEFRA consent. The harvest date is 18 Sep. 2019. After dehydrating in greenhouse, seeds were collected as 10 seeds/sample with 2 replicates from each line for FAMEs analysis.

FIG. 1c data showed that in DHAfae1 seeds, the 20:1Δ¹¹ accumulation is as low as 0.4%, indicating that all three homeologues of FAE1 were knocked out. Compared with parental line DHA1, the levels of palmitic acid (16:0) and stearic acid (18:0) were broadly the same, with the OA and LA levels slightly decreased by 1% and 2.5% respectively, whereas the ALA level was increased from 17.0% in DHA1 to 21.5% DHAfae1. This indicated that targeted mutagenesis of the FAE1 genes in DHA1 genetic background did not affect the OA precursor metabolites but did alter the downstream desaturation pathway as would be predicted. We then analysed the total C20+n-3 LC-PUFAs content in the different line. In DHA1fae1 seeds, the level of EPA was similar to that in DHA1, 9.1% compared to 9.3%. Interestingly the DHA level was modestly increased, from 9.7% in DHA1 to 12.6% in DHA1fae1. Eicosatetraenoic acid (ETA; 20:4Δ^{1,11,14,17}) and docosapentaenoic acid (DPA; 22:5Δ^{7,10,13,16,19}) levels followed similar trends with DHA, slightly enhanced by 1.3% and 1.5% respectively. In sum, total C20+n-3 fatty acids contents including ETA, EPA, DPA, DHA were increased by 5.5% from 27.5% in DHA1 to 33.0% in DHA1fae1. This is a significant increase, the level of which could not have been predicted from that data with DHA2015.1 or fae1 alone. This data demonstrated using the FAE1 mutant background results in an increase in n-3 LC-PUFAs production in the DHA1 line. This is surprising and unexpected because the ALA substrate is not known to have any role in the PUFA pathway

Studies by others have reported that the increased omega-6 (n-6) to n-3 ratio was highly pro-thrombotic and pro-inflammatory, and contributed to the prevalence of atherosclerosis, obesity, diabetes, and a wide range of inflammation disorders (Zárate et al., 2017). Therefore, we calculated the different n-3 and n-6 parameters (FIG. 1c). The total C20+n-6 fatty acids including dihomo-γ-linolenic acid (DGLA; 20:3Δ^{8,11,14}) and arachidonic acid (ARA; 20:4Δ^{5,8,11,14}) remained similar, at 3.1% in DHA1 and 2.8% in DHA1fae1, giving a ratio of C20+(n-3/n-6) as 8.8 in DHA1 and 11.7 in DHA1fae1. The total n-3 content was 47.5% in DHA1 and 58.2% in DHA1fae1, whereas the total n-6 content was 23.6% and 20.2% respectively. Therefore, the ratio of total (n-3/n-6) was 2.0 in DHA1 and 2.9 in DHA1fae1, which is also a significant increase compared with 2.2 in the fae1 mutant and 1.7 in both WT Celine and WT Suneson lines, indicating that the DHA1fae1 fatty acids have an even better health benefits than those of DHA1, fae1 and WTs. This is an unexpected benefit of combining DHA1 and FAE1, given that FAE1 elongates 18:1 to 20:1 and 22:1, and there was no evidence that varying the levels of 18:1 would be reflected in altered levels of EPA and DHA.

In conclusion, the combination of CRISPR-Cas9-gene-editing to inactivate the FAE1 pathway clearly results in a beneficial increase in the levels of EPA, DHA and other omega-3 LC-PUFAs in recombinant Camelina harbouring the DHA2015.1 cassette. In particular, the fae1 mutant not only is devoid of C20+ monounsaturated fatty acids (including the undesirable C22 erucic acid) but also has increased levels of omega-3 fatty acids such as ALA. Our previous studies have indicated that ALA is the primary endogenous fatty acid which is “consumed” to make EPA and DHA, and our data here further confirm this. This is in contrast to Canola, where recent attempts to engineer the accumulation of EPA and DHA result in the metabolism of the omega-6 precursor LA but not omega-3 ALA. In that respect, Canola is biased towards the synthesis of omega-6 fatty acids whereas Camelina is biased towards omega-3, requiring additional transgene-derived “push” in Canola to direct the flux of fatty acids on to the omega-3 track (discussed in Napier et al., 2018; see also FIG. 2 for pathways). It is also noteworthy that Canola already lacks the FAE1 activity, having been selected (by conventional plant breeding) for the absence of erucic acid which is present in parental varieties of Brassica napus seed oil. Collectively, these data suggest that Camelina is a superior host for the transgene-derived seed-specific synthesis of omega-3 LC-PUFAs such as EPA and DHA.

### Example IV: Fatty Acid Profile of Optimised Constructs

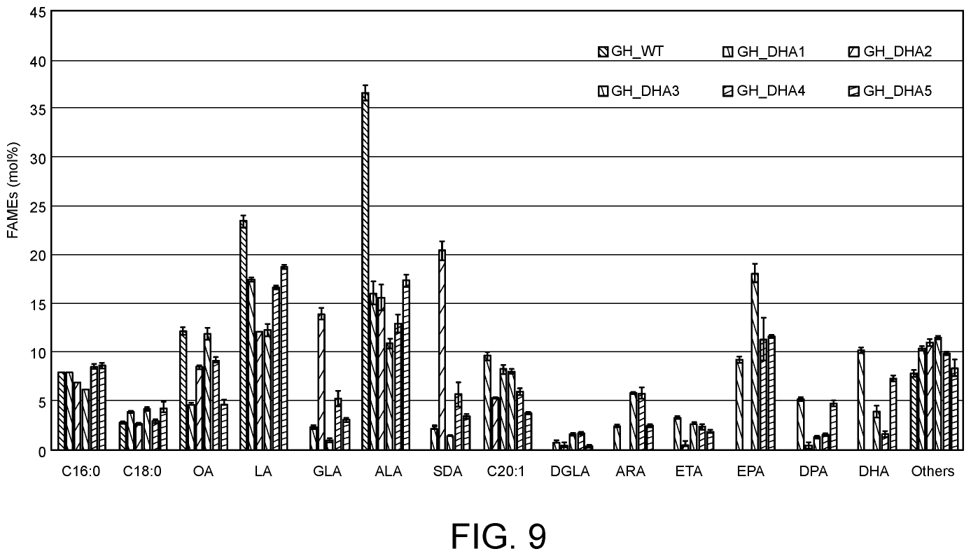

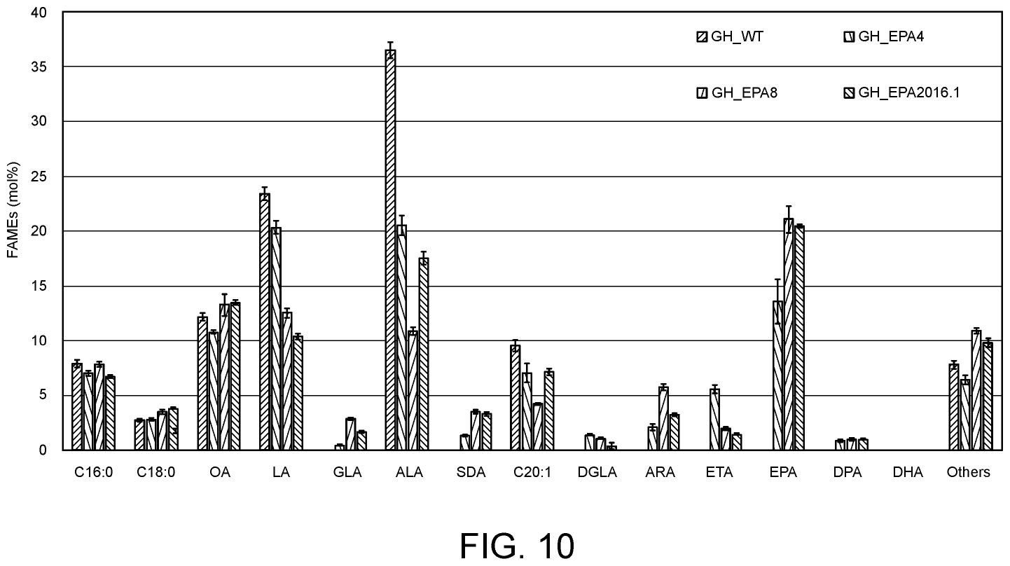

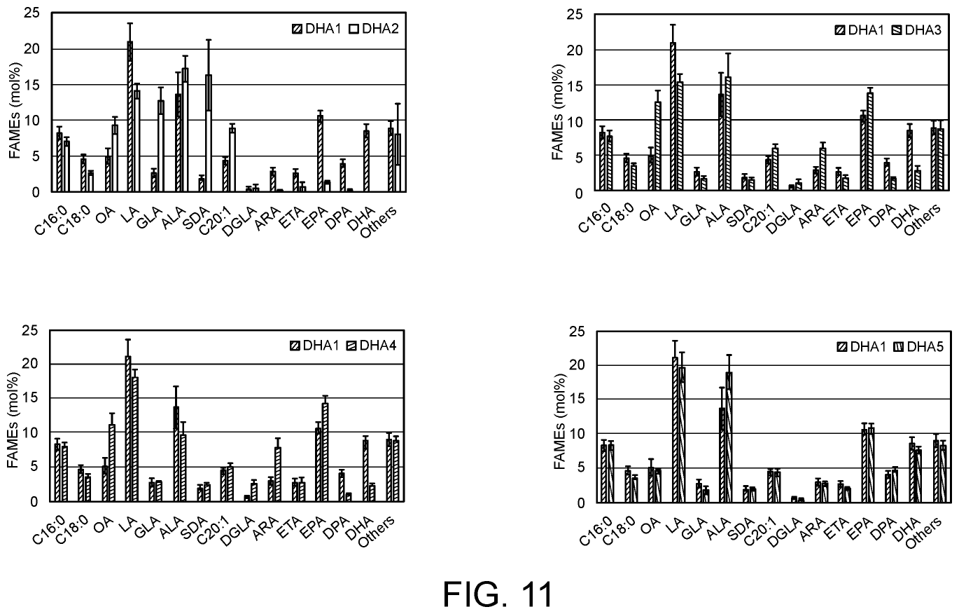

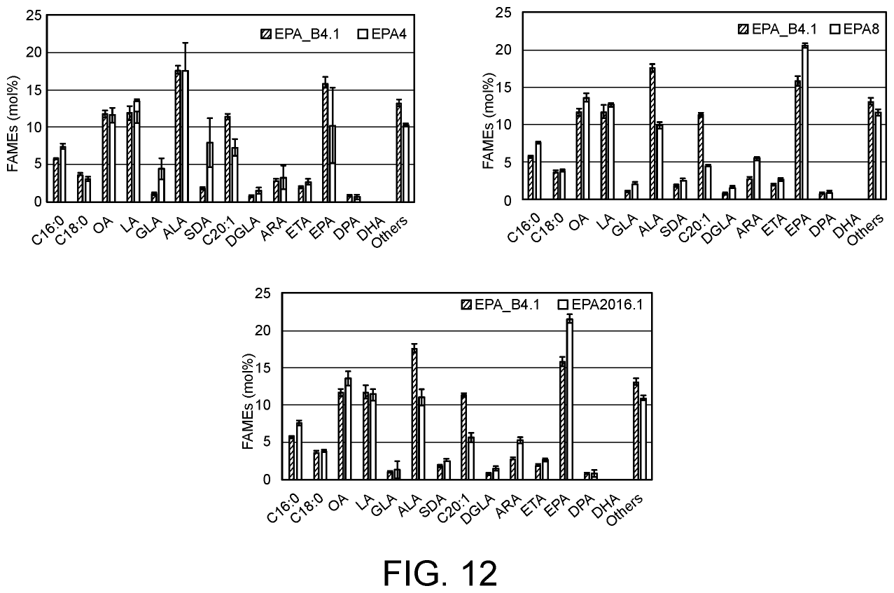

Based on the presence of target fatty acids EPA and DHA in GH-grown T2 material (FIG. 9; FIG. 10), a field-scale evaluation of these new iterations, benchmarked against the DHA2015.1 and EPA_B4.1 prototypes was undertaken. Approval to carry out an environmental release at the Rothamsted GM trial site in Harpenden was obtained from DEFRA (Consent 18/R8/01) and carried out as previously described (Usher et al., 2017). Plants were grown to maturity, seeds harvested by combining and their constituent fatty acids analysed by GC-FID (Usher et al., 2015), and compared with DHA2015.1 and EPA-B4.1 grown in the same trial in pairwise comparisons (FIGS. 11 and 12). In terms of non-native fatty acids, a number of observations are clear from this systematic analysis. Firstly, in the case of DHA2015.2 (the least complex modification), with just the replacement of the Piw3 ω3-desaturase with the Hpw3 activity, resulted in the undesirable accumulation of C18 D6-desaturated fatty acids and an absence of EPA and DHA. (FIG. 3; FIG. 7; see also FIG. 9 for side-by-side comparison of the different iterations). However, we believe that this phenomena is an event-specific example of transgene-silencing, most likely as a consequence of the site of insertion. Moreover, the same transgene and associated regulatory elements is present in other DHA2015 series constructs without the same over-accumulation of GLA and SDA (FIG. 9; cf. within FIG. 11). In the case of DHA2015.3 (FIG. 11B), the switching of the ELO5 activity to one derived from Ostreococcus RCC809 had impact on the levels of omega-3 LC-PUFAs, reducing the accumulation of C22 DPA (22:5n-3) and DHA with a concomitant build-up of EPA. This is likely due to poor elongation of EPA, as opposed to increased synthesis of that fatty acid, since DHA2015.3 also contains the replacement Hpw3 activity, which based on the elevated levels of ARAn-6, is less efficient at the conversion of omega-6 PUFAs to their omega-3 form, compared with the Piw3 activity present in DHA2015.1. In the case of DHA2015.4, the additional replacement of the terminal Δ4-desturase with an activity from T. pseudonana had a further deleterious impact on the accumulation of DHA, indicating that this new activity was inferior to the 0809Δ4 present in DHA2015.1. Collectively, these data indicate that the DHA2015.1 prototype is highly efficient at directing the synthesis of EPA and DHA, with minimal accumulation of undesired intermediates. It is worth noting that DHA2015.2-4 lacked the PsD12 desaturase, but the impact of the absence was not obvious. In a final iteration (DHA2015.5) had an additional activity of a FAD3 Δ15-desaturase added, resulting in the only new construct which matched or modestly outperformed the prototype over a number of generations, in terms of the accumulation of EPA, DPA and DHA (FIG. 7). DHA2015.5 produced increased levels of EPA and DPA, as well as C18 ALA, increasing the overall omega-3 levels of this seed oil.

In the case of the systematic improvements to the EPA construct EPA_B4.1, three iterations were evaluated (FIG. 4; FIG. 6—see also FIG. 12 for side-by-side comparisons). The simplest change (EPA2016.1) was the replacement of the OtD6 desaturase with the MsqD6 activity—this resulted in unexpectedly higher levels of EPA and a concomitant decrease in ALA, indicating that the MsqD6 was not only superior to OtD6, but also (unlike the latter), preferred omega-3 substrates (Sayanova et al., 2012).

The second iteration, EPA2015.4, additionally replaced the TcD5-desaturase with the similar EhuxD5 activity, though this resulted in the slight accumulation of 20:4n-3 indicating perhaps less efficient Δ5-desaturation of C20 elongation products. Overall, the performance of EPA2015.4 was inferior to the previous iteration and benchmark EPA_B4.1. The final variant, EPA2015.8, was analogous to DHA2015.5, with the addition of the PerfD15 activity to the EPA_B4.1 prototype—this gave a fatty acid profile with increased EPA, but unexpectedly, a reduction in ALA. However, this reduction in ALA could not assigned to the presence of the PerfD15 activity, since a similar reduction was also observed in EPA2016.1, which lacks this gene (FIG. 4). Interestingly, although all of the EPA series contain the Hpw3 activity instead of Piw3, there is some variation in the perceived impact of this activity and the conversion of ARA to EPA (see also comments below)—most noticeably more effective in EPA_B4.1 and EPA2015.4 than EPA2015.8 and EPA2016.1, the is no clear correlation as to the reason for this although difference in regulatory elements (promoters and terminators—Table 2) may partially explain this.

Based on the sum of all these data, several conclusions can be drawn. Firstly, based on the systematic replacement of all the activities present in DHA2015.1 and EPA_B4.1, the most impactful positive change is from the M. squamosa Δ6-desaturase whereas as substitutions with 0809Elo5, EhuxD5, and TpD4 all showed no improvement on the seed fatty acid profile compared to DHA2015.1. In the case of the MsqD6 desaturase, this high activity was more than a little surprising, since previous characterisation by others of the identical protein sequence had revealed a very low activity in Arabidopsis and an inability to direct the accumulation of any meaningful levels (<0.1% of EPA) in the seeds of these recombinant plants (Hoffman et al., 2008). Another unexpected change was as a consequence of using the Hpw3 activity instead of Piw3—not only did this result in reduced EPA and DHA, but also elevated arachidonic acid. Pathway models for the synthesis of EPA and DHA usually envisage the linear flow of substrates through both omega-6 and omega-3 “tracks” prior to ω3-desaturation as a final step but our data would indicate that biosynthetic intermediates such are ARA may also significantly contribute to the final levels of EPA and DHA via “track-changes”.

In conclusion, we present data from a systematic attempt to define and improve the individual activities which contribute to the most efficient production of omega-3 LC-PUFAs in recombinant camelina. It is interesting to note that the recombinant activity of genes derived from the same source organism do not necessarily all perform to the same level (e.g. 0809D4 vs 0809Elo5), supporting our “pick and mix” approach to combining the best activities from different organisms. Our studies reveal an improved combination of genes, as well as topics (such as the contribution of the Δ12 and Δ15 desaturases) for further studies. Finally, the benefit of carrying out field-based studies means our prototypes have already undergone initial validation as being “real-world-ready”.

TABLE 2
List of genes and elements used in the constructs

| | | Donor | |
|---|---|---|---|
| Element | Size | Organism | Description and Intended Function |
| RB | 24 bp | Agrobacterium | T-DNA Right border |
| | | tumefaciens | |
| LB | 23 bp | Agrobacterium | T-DNA Left border |
| | | tumefaciens | |
| Seed-specific | | | |
| promoters | | | |
| USP | 684 bp | Vicia faba | Unknown Seed Protein Seed-specific |
| | | | promoter |
| NP | 664 bp | Brassica | Napin seed specific promoter |
| | | napus | |
| CNL | 1064 bp | Linum | 2S seed storage protein (Conlinin) promoter |
| | | usitatissimum | |
| GLY | 702 bp | Glycine max | 11S Seed storage protein (Glycinin) |
| | | | promoter |
| SBP | 1800 bp | Arabidopsis | Sucrose-binding protein promoter (seed- |
| | | | specific) |
| PvArc | 1138 bp | Phaseolus | Arcelin-5 seed storage protein promoter |
| | | vulgaris | |
| Omega-3 | | | |
| biosynthetic | | | |
| genes | | | |
| OtΔ6 | 1665 bp | Synthetic | Encodes a fatty acid Δ6-desaturase from |
| | | | the marine picoalga Ostreococcus tauri |
| PSE1 | 873 bp | Synthetic | Encodes an acyl-CoA-dependent Δ6- |
| | | | elongase from the moss Physcomitrella |
| | | | patens |
| TcΔ5 | 1320 bp | Synthetic | Encodes a fatty acid Δ5-desaturase from |
| | | | the marine species Thraustochytrium |
| Piw3 | 1086 bp | Synthetic | Encodes a fatty acid w3-desaturase from |
| | | | Phytophora infestans |
| PsΔ12 | 1197 bp | Synthetic | Encodes a fatty acid Δ12-desaturase FAD2 |
| | | | activity from Phytophora sojae |
| OtElo5 | 903 bp | Synthetic | Encodes an acyl-CoA dependent Δ5- |
| | | | elongase from the marine picoalgae |
| | | | Ostreococcus tauri |
| TpΔ4 | 1653 bp | Synthetic | Encodes a fatty acid Δ4-desaturase from |
| | | | the marine diatom Thalassiosira |
| | | | pseudonana |
| O809Δ4 | 1480 bp | Synthetic | Encodes a fatty acid Δ4-desaturase from |
| | | | the marine picoalgae Ostreococcus RCC809 |
| MsqΔ6 | 1350 bp | Synthetic | Encodes a fatty acid Δ6-desaturase from |
| | | | the marine algae Mantoniella squamata |
| EmiD5 | 1368 bp | Synthetic | Encodes a fatty acid Δ5-desaturase from |
| | | | the marine algae Emiliania huxleyi |
| Hpw3 | 1086 bp | Synthetic | Encodes a fatty acid ω3-desaturase from |
| | | | Hyaloperonospora parasitica |
| PerfD15 | 1176 bp | Synthetic | Encodes a fatty acid Δ15-desaturase from |
| | | | Perilla frutescens |
| 809E5 | 906 bp | Synthetic | Encodes a fatty acid Δ5-elongase from the |
| | | | marine picoalgae Ostreococcus RCC809 |
| Marker genes | | | |
| DsRed | 684 bp | Synthetic | Encodes a florescent protein from |
| | | | Discosoma spp. |
| bar | 548 bp | Synthetic | Encodes phosphinothricin acetyl |
| | | | transferase, providing to resistance to Class |
| | | | H herbicides such as Basta ™, in which |
| | | | bialaphos is the active ingredient. |
| Regulatory | | | |
| elements | | | |
| p35S | 446 bp | Cauliflower | 35S promoter |
| | | mosaivc virus | |
| pNOS | 288 bp | Agrobacterium | Nopaline synthase gene promoter |
| | | tumefaciens | |
| NOSt | 256 bp | Agrobacterium | Nopaline synthase gene terminator |
| | | tumefaciens | sequence |
| OCSt | 192 bp | Agrobacterium | octopine synthase gene terminator |
| | | tumefaciens | sequence |
| 35St | 216 bp | Cauliflower | 35S transcript terminator sequence |
| | | mosaic virus | |
| CatpAt | 235 bp | Arabidopsis | Cathepsin A gene terminator sequence |
| | | thaliana | |
| Glyt | 451 bp | Glycine max | 11S storage protein (Glycinin) terminator |
| E9t | 558 bp | Arabidopsis | Ubiquitin E9 ligase gene terminator |
| | | thaliana | sequence |
| pCsVMV | 528 bp | Cassava vein | CsVMV promoter sequence |
| | | mosaic virus | |
| PvARCt | 600 bp | Phaseolus | Arcelin-5 gene terminator sequence |
| | | vulgaris | |
| HSPt | 245 bp | Arabidopsis | Heat shock protein 18.2 (HSP) gene |
| | | thaliana | terminator sequence |
| Phast | 183 bp | Phaseolus | Alpha-phaseolin gene terminator sequence |
| | | vulgaris | |
| Fad2t | 182 bp | Camelina | Microsomal oleate desaturases (FAD2) |
| | | sativa | gene terminator sequence |

### Example V

As shown previously with the GC-FID total fatty acid profiles of seeds from either WT, fae1, DHA2015.1 or fae1xDHA2015.1, these different genetic backgrounds and constructs combine to alter the overall seed fatty acid profile. For example, the fae1 mutation completely abolishes the accumulation of any C20+ fatty acids, resulting in a concomitant increase in C18 fatty acids, most notably ALA (from 36.9% to 47.3%). Similarly, the expression of the DHA2015.1 construct results in the synthesis of a number of non-native fatty acids, most notably EPA (9.3%), DPA (5.2%) and DHA (9.7%), as well as alterations to the levels of endogenous fatty acids (most obvious in the case of ALA, from 37% to 17%). When these two modifications are combined in the DHA1fae1 line, there is an unexpected synergist effect, with the elevated accumulation of C22 PUFAs such as DPA and DHA, although interestingly there was no significant difference in the accumulation of the levels of EPA.

Given these changes are manifest in the total seed fatty acid composition, it makes sense to look at the accumulation and distribution of the different fatty acids in main lipid type present in the seed, triacylglycerol (TAG). TAG is a neutral storage lipid and consists of a glycerol backbone onto which three fatty acids are esterified, with heterogeneity possible at each position. Thus, given that the seed fatty acid profile consists of at least 10 different acyl chains, then the potential number of different TAG species is very high (10³), and this variation can also serve as a diagnostic fingerprint. In a plant species such as camelina, the number of individual TAGs present in the seed oil has been shown by us to be ˜80, with considerable variation in the abundance of specific TAG species. We have also shown that transgene-derived accumulation of EPA, DPA and DHA expands the repertoire of TAGs present in the seed. We therefore used LC-MS methods to determine the TAG profiles of the seed lipids from WT, fae1, DHA1 and DHA1fae plants, to provide a deeper analyses of the changes to both fatty acid profile and lipid metabolism in these plants. Moreover, since the ultimate goal of these experiments is to modify the oil (i.e. the TAG) composition of the seeds, such data can help inform and improve the accumulation of EPA, DPA and DHA in recombinant camelina seeds. FIG. 13a shows a comparison of the TAG species present in the seeds of DHA2015.1 and fae1DHA2015.1—the individual TAG species are resolved by the mass-spec on the basis of the number of carbon atoms and the number of double bonds—for example “56:6” indicates a TAG of 56 carbons and 6 double bonds. Bearing in mind that this figure is the sum of all three fatty acids that are present on the glycerol backbone, this analysis does not give an unambiguous identification since the composition 56:6 could be generated by a number of different permutations (e.g. 18:3+18:3+20:0, 18:3+18:2+20:1, etc) although inferences can be made based on the abundance of the particular TAG species and also the levels of the predicted fatty acids in the total seed fatty acids. As shown in FIG. 13a, there is a striking difference (shift) in the profile of the TAGs present in fae1 versus DHA1 versus fae1 DHA1, with the most abundant TAGs in fae1 being of the C54 family and ranging from 3-9 unsaturations—this is commensurate with TAGs rich in ALA (18:3) and LA (18:2). Note the almost complete absence of any TAG species of C56+, indicative of the absence of C20+ fatty acids (as shown in FIG. 1 and commensurate with the fae1 mutation). In the case of DHA1 and fae1 DHA1, many more TAG species are present, most noticeably of the C58 family, but ranging from C56-C66. Almost all of these TAG species present in DHA1 and fae1 DHA1 are absent in the parental fae1 background. That these TAG species are the consequence of the DHA1 transgene is confirmed in FIG. 13b, which superimposes the TAG profile for WT Camelina (two different cultivars), confirming that the TAGs found in DHA1 and fae1 DHA1 are absent in WT.

FIG. 13c is a closer look at the pattern of accumulation of these novel (DHA1-specific) TAGs in either the normal WT background or the genome-edited fae1 mutant background. As can be seen in this side-by-side comparison, the overall number of TAG species present in the two different backgrounds is not the same, with additional TAG species being present in fae1DHA1 (e.g. 64:17, 66:17) and a number of TAG species being markedly upregulated or down-regulated in the case of fae1 DHA1. It is of particular interest that TAGs such as 58:8-58:12 are more abundant, indicative of elevated accumulated of long chain polyunsaturated fatty acids in TAG.

To provide further information of as to the fatty composition of individual TAG species, it is possible to investigate the molecular fragmentation pattern of the ionized compounds in the mass spec. Using this approach, it is possible to estimate the abundance of fatty acids of interest in particular TAG species—this is exemplified in FIG. 14a, where the presence of EPA (20:5) was determined in both the DHA1 and fae1 DHA1 samples. This revealed a number of EPA-TAG species present only in the fae1 DHA1 sample, including 56:8 and 60:12, amongst others. This latter TAG species could comprise EPA (20:5)+DHA (22:6)+18:1 (see slide G). There are also a number of longer chain C62-4 TAGs that contain EPA in the fae1 background but are absent in the regular WT background.

In the case of identifying TAGs containing DPA (22:5) (FIG. 14b), a number of low abundance but distinctive C60+ polyunsaturated (11+ double bonds) were identified in the fae1 DHA1 background that were absent in the DHA1 line, although it would not have been possible to predict such a profile in the absence of experimental data.

Finally, in the case of identifying TAGs containing DHA (FIG. 14c), multiple long carbon (C62+) PUFA TAGs were identified (e.g. 66:16, 66:17)—66:17 being DHA+DHA+DPA (22:6+22:6+22:5), and corroborated by the identification of 66:17 as a DPA-containing TAG in FIG. 4B. Similarly, 60:12, only identified in fae1DHA1, is shown to contain both DHA (FIG. 4C) and EPA (FIG. 4A), confirming that this is a TAG which contains two omega-3 LC-PUFAs as well as oleic acid (18:1).

Collectively these data confirm the benefits of the fae1 mutation on the accumulation of desirable omega-3 LC-PUFAs such as EPA and DHA, and also (importantly) their incorporation in a family of TAG species that is quite different from that obtained when using the wildtype FAE1 background. Specific benefits include the generation of TAGs containing DHA and DPA (66:17) as well as DHA and EPA (60:12).

### Example VI: Astaxanthin Constructs

| | | Donor | |
|---|---|---|---|
| Element | Size | Organism | Description and Intended Function |
| RB | 24 bp | Agrobacterium | T-DNA Right border |
| | | tumefaciens | |
| LB | 23 bp | Agrobacterium | T-DNA Left border |
| | | tumefaciens | |
| Seed-specific | | | |
| promoters | | | |
| GLY | 702 bp | Glycine max | 11S Seed storage protein (Glycinin) |
| | | | promoter |
| Astaxanthin | | | |
| biosynthetic | | | |
| genes | | | |
| ZmPhys | 1232 bp | Synthetic | Encodes phytoene synthase from maize |
| HBFD1 | 1226 bp | Synthetic | Encodes carotenoid 4-hydroxy-β-ring 4- |
| | | | dehydrogenase from Adonis aestivalis |
| CBFD2 | 687 bp | Synthetic | Encodes carotenoid β-ring 4- |
| | | | dehydrogenase from Adonis aestivalis |
| Marker genes | | | |
| bar | 548 bp | Synthetic | Encodes phosphinothricin acetyl |
| | | | transferase, providing to resistance to |
| | | | Class H herbicides such as Basta ™, in |
| | | | which bialaphos is the active ingredient. |
| Regulatory | | | |
| elements | | | |
| pNOS | 288 bp | Agrobacterium | Nopaline synthase gene promoter |
| | | tumefaciens | |
| NOSt | 256 bp | Agrobacterium | Nopaline synthase gene terminator |
| | | tumefaciens | sequence |

In Construct ASX-Δ2 (as shown in FIG. 16), three heterologous genes under the control of seed-specific promoters were assembled to direct the synthesis of the ketocarotenoid astaxanthin in the seeds of transgenic C. sativa. A phytoene synthase from maize (ZmPhys) was introduced to enhance the accumulation of phytoene (by the conversion of geranylgeranyl-diphosphate to phytoene) and subsequently to b-carotene, the latter to provide substrate for the carotenoid β-ring 4-dehydrogenase (CBFD2) from Adonis aestivalis. The product of that enzyme, a 4-hydroxy-β-ring, is then converted to astaxanthin by the 4-hydroxy-β-ring 4-dehydrogenase (HBFD1) from Adonis aestivalis. All three genes are synthetic, being codon-optimised from their native sequences (derived either from maize or Adonis aestivalis). Each synthetic coding sequence is regulated by an individual seed-specific promoter, and also defined by a transcription termination sequence. Within the T-DNA there is also the selectable marker, BAR, which is constitutively expressed under the control of the nopaline synthase promoter and terminator. This T-DNA region has been introduced into transgenic C. sativa by A. tumefaciens-mediated transformation as described above for DHA2015.1, and primary transgenic events identified by their resistance to bialaphos (glufosinate-ammonium). To check for the presence of astaxanthin accumulation, astaxanthin was extracted from seeds with acetone and the absorbance of the extract was measured at 475 nm. This was compared to a standard curve constructed using commercially available astaxanthin. Seeds from lines which show astaxanthin accumulation were then sown on ½ M+S plates containing 300 ug/ml glufosinate-ammonium and survivors were transferred to soil. The traits encoding in the ASX-Δ2 construct are inherited in a Mendelian fashion indicative of a single insertion, and have been shown to be stable to the T5 generation, at which point they were used for crossing with the DHA2015.1 #39 event.

### Example VII

In this example, we show the results of a cross between the fae1 mutant background and a second PUFA accumulating line (EPA2105.8, which is described above). As can be seen from the data in FIG. 17, the fae1 background has a significant increase in the level of EPA compared to the expression of this cassette in the WT background.

A schematic of the EPA2015.8 construct is shown in FIG. 17. Of note, this construct contains the PerfD15 desaturase.

### Example VIII

In this example, we show the results of a cross between ASX (astaxanthin), DHA1 and fae1 lines. As shown in the below table, and in FIG. 18a, the astaxanthin concentration was significantly higher in the CASX lines (DHA1×ASX) compared to wild-type or the DHA1 or fae1 lines. Of note, the concentration of astaxanthin was even further increased in the fae1×CASX lines. Astaxanthin was diluted in 0.5 ml acetone for nanodrop 475 nm absorbance assay.

TABLE 3

| | 475 nm | Astaxanthin concentration |
|---|---|---|
| Sample name | absorbance | (ug/g in seeds) |
| WT Celine-1 | 0.095 | 5.12 |
| WT Celine-2 | 0.083 | 4.49 |
| DHA1-1 | 0.034 | 1.91 |
| DHA1-2 | 0.026 | 1.49 |
| fae1-1 | 0.026 | 1.49 |
| fae1-2 | 0.024 | 1.39 |
| ASX-1 | 1.04 | 54.86 |
| ASX-2 | 0.962 | 50.75 |
| CASX-1 | 0.917 | 48.38 |
| CASX-2 | 1.122 | 59.17 |
| F2 fae1 × CASX | 1.76 | 92.75 |
| (Cross-1) | | |
| F2 fae1 × CASX | 1.851 | 97.53 |
| (Cross-2) | | |

We further performed a GC-FID analysis of seeds from the F2 cross between fae1 and CASX line (which was generated by previous crossing between DHA2015.1 and ASX-2). As shown in FIG. 18b, the fatty acid profiles confirm the presence of the DHA2015.1 transgenes (indicated by the presence of EPA, DPA and DHA), and the background is clearly fae1 mutant, since the levels of 20:1 and other saturated and monounsaturated fatty acids are reduced. These seeds have been visualized scored as containing astaxanthin and related ketocarotenoids and also through the analysis of pooled seeds (as described above; FIG. 18a). Note that although the three traits (fae1, ASX, DHA1) are all segregating independently in this F2 population, all three manifest themselves in non-homozygous states.

### REFERENCES

Betancor M B, Li K, Bucerzan V S, Sprague M, Sayanova O, Usher S, Han L et al. (2018) Oil from recombinant Camelina sativa containing over 25% n-3 long-chain PUFA as the major lipid source in feed for Atlantic salmon (Salmo salar). Br J Nutr., 119, 1378-1392
Han L, Usher S, Sandgrind S, Hassall K, Sayanova O, Michaelson L V, Haslam R P et al. (2020) High level accumulation of EPA and DHA in field-grown recombinant Camelina—a multi-territory evaluation of TAG accumulation and heterogeneity. Plant Biotechnol. J., pp. 1-12
Napier, J. A., Olsen, R. E., & Tocher, D. R. (2019). Update on GM canola crops as novel sources of omega-3 fish oils. Plant biotechnology journal, 17(4), 703.
Napier J A, Sayanova O. (2020) Nutritional enhancement in plants—green and greener. Curr Opin Biotechnol., 61:122-127
Ozseyhan M E, Kang J, Mu X, Lu C. (2018) Mutagenesis of the FAE1 genes significantly changes fatty acid composition in seeds of Camelina sativa. Plant Physiol Biochem., 123:1-7.
Petrie J R, Shrestha P, Belide S, Kennedy Y, Lester G, Liu Q, Divi U K. (2014) Metabolic engineering Camelina sativa with fish oil-like levels of DHA. PLoS One, 9(1): e85061
Ruiz-Lopez N, Haslam R P, Napier J A, Sayanova O. (2014) Successful high-level accumulation of fish oil omega-3 long-chain polyunsaturated fatty acids in a recombinant oilseed crop. Plant J., 77, 198-208
Usher S, Han L, Haslam R P, Michaelson L V, Sturtevant D, Aziz M, Chapman K D. (2017) Tailoring seed oil composition in the real world: optimising omega-3 long chain polyunsaturated fatty acid accumulation in recombinant Camelina sativa. Sci Rep., 7(1):6570
Zárate R, El Jaber-Vazdekis N, Tejera N, Pérez JA, Rodríguez C. (2017) Significance of long chain polyunsaturated fatty acids in human health. Clin Transl Med., 6:25
Tocher D R, Betancor M B, Sprague M, Olsen R E, Napier J A. (2019) Omega-3 Long-Chain Polyunsaturated Fatty Acids, EPA and DHA: Bridging the Gap between Supply and Demand. Nutrients, 11, 89
West A L, Miles E A, Lillycrop K A, Han L, Sayanova O, Napier J A, Calder P C et al. (2019) Postprandial incorporation of EPA and DHA from recombinant Camelina sativa oil into blood lipids is equivalent to that from fish oil in healthy humans. Br J Nutr., 121(11): 1235-1246
Lim, Keng Chin et al. “Astaxanthin as feed supplement in aquatic animals.” Reviews in Aquaculture 10 (2018): 738-773.

| SEQUENCE LISTING | |
|---|---|
| (Codon-optimised OtD6 Δ6-desaturase from | |
| Ostreococcus tauri) | |
| SEQ ID NO: 1 | |
| 1 | ATGTGTGTTGAGACCGAGAACAACGATGGAATCCCTACTGTGGAGATCGCTTTCGATGGA |
| 61 | GAGAGAGAAAGAGCTGAGGCTAACGTGAAGTTGTCTGCTGAGAAGATGGAACCTGCTGCT |
| 121 | TTGGCTAAGACCTTCGCTAGAAGATACGTGGTTATCGAGGGAGTTGAGTACGATGTGACC |
| 181 | GATTTCAAACATCCTGGAGGAACCGTGATTTTCTACGCTCTCTCTAACACTGGAGCTGAT |
| 241 | GCTACTGAGGCTTTCAAGGAGTTCCACCACAGATCTAGAAAGGCTAGGAAGGCTTTGGCT |
| 301 | GCTTTGCCTTCTAGACCTGCTAAGACCGCTAAAGTGGATGATGCTGAGATGCTCCAGGAT |
| 361 | TTCGCTAAGTGGAGAAAGGAGTTGGAGAGGGACGGATTCTTCAAGCCTTCTCCTGCTCAT |
| 421 | GTTGCTTACAGATTCGCTGAGTTGGCTGCTATGTACGCTTTGGGAACCTACTTGATGTAC |
| 481 | GCTAGATACGTTGTGTCCTCTGTGTTGGTTTACGCTTGCTTCTTCGGAGCTAGATGTGGA |
| 541 | TGGGTTCAACATGAGGGAGGACATTCTTCTTTGACCGGAAACATCTGGTGGGATAAGAGA |
| 601 | ATCCAAGCTTTCACTGCTGGATTCGGATTGGCTGGATCTGGAGATATGTGGAACTCCATG |
| 661 | CACAACAAGCACCATGCTACTCCTCAAAAAGTGAGGCACGATATGGATTTGGATACCACT |
| 721 | CCTGCTGTTGCTTTCTTCAACACCGCTGTGGAGGATAATAGACCTAGGGGATTCTCTAAG |
| 781 | TACTGGCTCAGATTGCAAGCTTGGACCTTCATTCCTGTGACTTCTGGATTGGTGTTGCTC |
| 841 | TTCTGGATGTTCTTCCTCCATCCTTCTAAGGCTTTGAAGGGAGGAAAGTACGAGGAGCTT |
| 901 | GTGTGGATGTTGGCTGCTCATGTGATTAGAACCTGGACCATTAAGGCTGTTACTGGATTC |
| 961 | ACCGCTATGCAATCCTACGGACTCTTCTTGGCTACTTCTTGGGTTTCCGGATGCTACTTG |
| 1021 | TTCGCTCACTTCTCTACTTCTCACACCCATTTGGATGTTGTTCCTGCTGATGAGCATTTG |
| 1081 | TCTTGGGTTAGGTACGCTGTGGATCACACCATTGATATCGATCCTTCTCAGGGATGGGTT |
| 1141 | AACTGGTTGATGGGATACTTGAACTGCCAAGTGATTCATCACCTCTTCCCTTCTATGCCT |
| 1201 | CAATTCAGACAACCTGAGGTGTCCAGAAGATTCGTTGCTTTCGCTAAGAAGTGGAACCTC |
| 1261 | AACTACAAGGTGATGACTTATGCTGGAGCTTGGAAGGCTACTTTGGGAAACCTCGATAAT |
| 1321 | GTGGGAAAGCACTACTACGTGCACGGACAACATTCTGGAAAGACCGCTTGA |
| (OtD6 Δ6-desaturase from Ostreococcus tauri) | |
| SEQ ID NO: 2 | |
| MCVETENNDGIPTVEIAFDGERERAEANVKLSAEKMEPAALAKTFARRYVVIEGVEYDVT | |
| DFKHPGGTVIFYALSNTGADATEAFKEFHHRSRKARKALAALPSRPAKTAKVDDAEMLQD | |
| FAKWRKELERDGFFKPSPAHVAYRFAELAAMYALGTYLMYARYVVSSVLVYACFFGARCG | |
| WVQHEGGHSSLTGNIWWDKRIQAFTAGFGLAGSGDMWNSMHNKHHATPQKVRHDMDLDTT | |
| PAVAFFNTAVEDNRPRGFSKYWLRLQAWTFIPVTSGLVLLFWMFFLHPSKALKGGKYEEL | |
| VWMLAAHVIRTWTIKAVTGFTAMQSYGLFLATSWVSGCYLFAHFSTSHTHLDVVPADEHL | |
| SWVRYAVDHTIDIDPSQGWVNWLMGYLNCQVIHHLFPSMPQFRQPEVSRRFVAFAKKWNL | |
| NYKVMTYAGAWKATLGNLDNVGKHYYVHGQHSGKTA* | |
| (Codon-optimised PSE1 Δ6-elongase from | |
| Physcomitrella patens) | |
| SEQ ID NO: 3 | |
| 1 | ATGGAAGTTGTTGAGAGGTTCTACGGAGAGTTGGATGGAAAGGTTTCCCAAGGAGTGAAC |
| 61 | GCTTTGTTGGGATCTTTCGGAGTTGAGTTGACTGATACCCCAACTACTAAGGGATTGCCA |
| 121 | CTCGTTGATTCTCCAACTCCAATTGTGTTGGGAGTGTCTGTTTACTTGACCATCGTGATC |
| 181 | GGAGGATTGCTTTGGATCAAGGCTAGAGATCTCAAGCCAAGAGCTTCTGAGCCATTCTTG |
| 241 | TTGCAAGCTTTGGTGTTGGTGCACAACTTGTTCTGCTTCGCTTTGTCTCTTTACATGTGC |
| 301 | GTGGGTATCGCTTACCAAGCTATCACCTGGAGATATTCCTTGTGGGGAAACGCTTATAAC |
| 361 | CCAAAGCACAAGGAGATGGCTATCCTCGTTTACCTCTICTACATGTCCAAGTACGTGGAG |
| 421 | TTCATGGATACCGTGATCATGATCCTCAAGAGATCCACCAGACAGATTTCTTTCCTCCAC |
| 481 | GTGTACCACCATTCTTCTATCTCCCTTATCTGGTGGGCTATTGCTCATCATGCTCCAGGA |
| 541 | GGAGAGGCTTATTGGAGTGCTGCTCTCAACTCTGGAGTGCATGTGTTGATGTACGCTTAC |
| 601 | TACTTCTTGGCTGCTTGCTTGAGATCTTCCCCAAAGCTCAAGAACAAGTACCTCTTCTGG |
| 661 | GGAAGATACCTCACCCAATTCCAGATGTTCCAGTTCATGCTCAACTTGGTGCAAGCTTAC |
| 721 | TACGATATGAAAACCAACGCTCCATATCCACAATGGCTCATCAAGATCCTCTTCTACTAC |
| 781 | ATGATCTCCCTCTTGTTCCTCTTCGGAAACTTCTACGTGCAAAAGTACATCAAGCCATCC |
| 841 | GATGGAAAGCAAAAGGGAGCTAAGACCGAGTGA |
| (PSE1 Δ6-elongase from Physcomitrella patens) | |
| SEQ ID NO: 4 | |
| MEVVERFYGELDGKVSQGVNALLGSFGVELTDTPTTKGLPLVDSPTPIVLGVSVYLTIVI | |
| GGLLWIKARDLKPRASEPFLLQALVLVHNLFCFALSLYMCVGIAYQAITWRYSLWGNAYN | |
| PKHKEMAILVYLFYMSKYVEFMDTVIMILKRSTRQISFLHVYHHSSISLIWWAIAHHAPG | |
| GEAYWSAALNSGVHVLMYAYYFLAACLRSSPKLKNKYLFWGRYLTQFQMFQFMLNLVQAY | |
| YDMKTNAPYPQWLIKILFYYMISLLFLFGNFYVQKYIKPSDGKQKGAKTE* | |
| (Codon-optimised TcΔ5-desaturase from | |
| Thraustochytrium sp.) | |
| SEQ ID NO: 5 | |
| 1 | ATGGGAAAAGGATCTGAGGGAAGATCTGCTGCTAGAGAGATGACTGCTGAGGCTAACGGA |
| 61 | GATAAGAGAAAGACCATCCTCATTGAGGGAGTGTTGTACGATGCTACCAACTTCAAACAC |
| 121 | CCAGGAGGTTCCATTATTAACTTCCTCACCGAGGGAGAAGCTGGAGTTGATGCTACCCAA |
| 181 | GCTTACAGAGAGTTCCATCAGAGATCCGGAAAGGCTGATAAGTACCTCAAGTCCCTCCCA |
| 241 | AAGTTGGATGCTTCTAAGGTGGAGTCTAGGTTCTCTGCTAAGGAGCAGGCTAGAAGGGAC |
| 301 | GCTATGACCAGGGATTACGCTGCTTTCAGAGAGGAGTTGGTTGCTGAGGGATACTTCGAT |
| 361 | CCATCTATCCCACACATGATCTACAGAGTGGTGGAGATTGTGGCTTTGTTCGCTTTGTCT |
| 421 | TTCTGGTTGATGTCTAAGGCTTCTCCAACCTCTTTGGTTTTGGGAGTGGTGATGAACGGA |
| 481 | ATCGCTCAAGGAAGATGCGGATGGGTTATGCATGAGATGGGACACGGATCTTTCACTGGA |
| 541 | GTTATCTGGCTCGATGATAGGATGTGCGAGTTCTTCTACGGAGTTGGATGTGGAATGTCT |
| 601 | GGACACTACTGGAAGAACCAGCATTCTAAGCACCATGCTGCTCCAAACAGATTGGAGCAC |
| 661 | GATGTGGATTTGAACACCTTGCCACTCGTTGCTTTCAACGAGAGAGTTGTGAGGAAGGTT |
| 721 | AAGCCAGGATCTTTGTTGGCTTTGTGGCTCAGAGTTCAGGCTTATTTGTTCGCTCCAGTG |
| 781 | TCTTGCTTGTTGATCGGATTGGGATGGACCTTGTACTTGCACCCAAGATATATGCTCAGG |
| 841 | ACCAAGAGACATATGGAGTTTGTGTGGATCTTCGCTAGATATATCGGATGGTTCTCCTTG |
| 901 | ATGGGAGCTTTGGGATATTCTCCTGGAACTTCTGTGGGAATGTACCTCTGCTCTTTCGGA |
| 961 | CTTGGATGCATCTACATCTTCCTCCAATTCGCTGTGTCTCATACCCATTTGCCAGTTACC |
| 1021 | AACCCAGAGGATCAATTGCATTGGCTTGAGTACGCTGCTGATCATACCGTGAACATCTCT |
| 1081 | ACCAAGTCTTGGTTGGTTACCTGGTGGATGTCTAACCTCAACTTCCAAATCGAGCATCAT |
| 1141 | TTGTTCCCAACCGCTCCACAATTCAGGTTCAAGGAGATCTCTCCAAGAGTTGAGGCTCTC |
| 1201 | TTCAAGAGACATAACCTCCCTTACTACGATTTGCCATACACCTCTGCTGTTTCTACTACC |
| 1261 | TTCGCTAACCTCTACTCTGTTGGACATTCTGTTGGAGCTGATACCAAGAAGCAGGATTGA |
| (TcΔ5-desaturase from Thraustochytrium sp.) | |
| SEQ ID NO: 6 | |
| MGKGSEGRSAAREMTAEANGDKRKTILIEGVLYDATNFKHPGGSIINFLTEGEAGVDATQ | |
| AYREFHRSGKADKYLKSLPKLDASKVESRFSAKEQARRDAMTRDYAAFREELVAEGYFDP | |
| SIPHMIYRVVEIVALFALSFWLMSKASPTSLVLGVVMNGIAQGRCGWVMHEMGHGSFTGV | |
| IWLDDRMCEFFYGVGCGMSGHYWKNQHSKHHAAPNRLEHDVDLNTLPLVAFNERVVRKVK | |
| PGSLLALWLRVQAYLFAPVSCLLIGLGWTLYLHPRYMLRTKRHMEFVWIFARYIGWFSLM | |
| GALGYSPGTSVGMYLCSFGLGCIYIFLQFAVSHTHLPVINPEDQLHWLEYAADHTVNIST | |
| KSWLVTWWMSNLNFQIEHHLFPTAPQFRFKEISPRVEALFKRHNLPYYDLPYTSAVSTTF | |
| ANLYSVGHSVGADTKKQD* | |
| (Codon-optimised OtELo5 Δ5-elongase from | |
| Ostreococcus tauri) | |
| SEQ ID NO: 7 | |
| 1 | ATGTCTGCTTCTGGAGCTTTGTTGCCTGCTATTGCTTTCGCTGCTTACGCTTACGCTACC |
| 61 | TACGCTTATGCTTTCGAGTGGTCTCATGCTAACGGAATCGATAACGTGGATGCTAGAGAG |
| 121 | TGGATTGGAGCTTTGTCTTTGAGACTCCCTGCAATTGCTACCACCATGTACCTCTTGTTC |
| 181 | TGCCTTGTGGGACCTAGATTGATGGCTAAGAGGGAGGCTTTTGATCCTAAGGGATTCATG |
| 241 | CTCGCTTACAACGCTTACCAAACCGCTTTCAACGTTGTGGTGCTCGGAATGTTCGCTAGA |
| 301 | GAGATCTCTGGATTGGGACAACCTGTTTGGGGATCTACTATGCCTTGGAGCGATAGGAAG |
| 361 | TCCTTCAAGATTTTGTTGGGAGTGTGGCTCCATTACAACAATAAGTACCTCGAGTTGTTG |
| 421 | GATACTGTGTTCATGGTGGCTAGGAAAAAGACCAAGCAGCTCTCTTTCTTGCATGTGTAC |
| 481 | CATCATGCTTTGTTGATTTGGGCTTGGTGGCTTGTTTGTCATCTCATGGCTACCAACGAT |
| 541 | TGCATCGATGCTTATTTCGGAGCTGCTTGCAACTCTTTCATCCACATCGTGATGTACTCC |
| 601 | TACTACCTCATGTCTGCTTTGGGAATTAGATGCCCTTGGAAGAGATATATCACCCAGGCT |
| 661 | CAGATGTTGCAATTCGTGATCGTGTTCGCTCATGCTGTTTTCGTGCTCAGACAAAAGCAC |
| 721 | TGCCCTGTTACTTTGCCTTGGGCACAAATGTTCGTGATGACAAATATGTTGGTGCTCTTC |
| 781 | GGAAACTTCTACCTCAAGGCTTACTCTAACAAGTCTAGGGGAGATGGAGCTTCTTCTGTT |
| 841 | AAGCCTGCTGAGACTACTAGAGCACCTTCTGTGAGAAGAACCAGGTCCAGGAAGATCGAT |
| 901 | TGA |
| (OtELo5 Δ5-elongase from Ostreococcus tauri) | |
| SEQ ID NO: 8 | |
| MSASGALLPAIAFAAYAYATYAYAFEWSHANGIDNVDAREWIGALSLRLPAIATTMYLLF | |
| CLVGPRLMAKREAFDPKGFMLAYNAYQTAFNVVVLGMFAREISGLGQPVWGSTMPWSDRK | |
| SFKILLGVWLHYNNKYLELLDTVFMVARKKTKQLSFLHVYHHALLIWAWWLVCHLMATND | |
| CIDAYFGAACNSFIHIVMYSYYLMSALGIRCPWKRYITQAQMLQFVIVFAHAVFVLRQKH | |
| CPVTLPWAQMFVMTNMLVLFGNFYLKAYSNKSRGDGASSVKPAETTRAPSVRRTRSRKI | |
| D* | |
| (Codon-optimised PsΔ12-desaturase from | |
| Phytophthora sojae) | |
| SEQ ID NO: 9 | |
| 1 | ATGGCTATTTTGAACCCTGAGGCTGATTCTGCTGCTAACCTCGCTACTGATTCTGAGGCT |
| 61 | AAGCAAAGACAATTGGCTGAGGCTGGATACACTCATGTTGAGGGTGCTCCTGCTCCTTTG |
| 121 | CCTTTGGAGTTGCCTCATTTCTCTCTCAGAGATCTCAGAGCTGCTATTCCTAAGCACTGC |
| 181 | TTCGAGAGATCTTTCGTGACCTCCACCTACTACATGATCAAGAACGTGTTGACTTGCGCT |
| 241 | GCTTTGTTCTACGCTGCTACCTTCATTGATAGAGCTGGAGCTGCTGCTTATGTTTTGTGG |
| 301 | CCTGTGTACTGGTTCTTCCAGGGATCTTACTTGACTGGAGTGTGGGTTATCGCTCATGAG |
| 361 | TGTGGACATCAGGCTTATTGCTCTTCTGAGGTGGTGAACAACTTGATTGGACTCGTGTTG |
| 421 | CATTCTGCTTTGTTGGTGCCTTACCACTCTTGGAGAATCTCTCACAGAAAGCACCATTCC |
| 481 | AACACTGGATCTTGCGAGAACGATGAGGTTTTCGTTCCTGTGACCAGATCTGTGTTGGCT |
| 541 | TCTTCTTGGAACGAGACCTTGGAGGATTCTCCTCTCTACCAACTCTACCGTATCGTGTAC |
| 601 | ATGTTGGTTGTTGGATGGATGCCTGGATACCTCTTCTTCAACGCTACTGGACCTACTAAG |
| 661 | TACTGGGGAAAGTCTAGGTCTCACTTCAACCCTTACTCCGCTATCTATGCTGATAGGGAG |
| 721 | AGATGGATGATCGTGCTCTCCGATATTTTCTTGGTGGCTATGTTGGCTGTTTTGGCTGCT |
| 781 | TTGGTGCACACTTTCTCCTTCAACACCATGGTGAAGTTCTACGTGGTGCCTTACTTCATT |
| 841 | GTGAACGCTTACTTGGTGTTGATTACCTACCTCCAACACACCGATACCTACATCCCTCAT |
| 901 | TTCAGAGAGGGAGAGTGGAATTGGTTGAGAGGAGCTTTGTGCACTGTGGATAGATCATTT |
| 961 | GGTCCATTCCTCGATTCTGTGGTGCATAGAATCGTGGATACCCATGTTTGCCACCACATC |
| 1021 | TTCTCCAAGATGCCTTTCTATCATTGCGAGGAGGCTACCAACGCTATTAAGCCTCTCCTC |
| 1081 | GGAAAGTTCTACTTGAAGGATACCACTCCTGTTCCTGTTGCTCTCTGGAGATCTTACACC |
| 1141 | CATTGCAAGTTCGTTGAGGATGATGGAAAGGTGGTGTTCTACAAGAACAAGCTCTAG |
| (PsΔ12-desaturase from Phytophthora sojae) | |
| SEQ ID NO: 10 | |
| MAILNPEADSAANLATDSEAKORQLAEAGYTHVEGAPAPLPLELPHFSLRDLRAAIPKHC | |
| FERSFVTSTYYMIKNVLTCAALFYAATFIDRAGAAAYVLWPVYWFFQGSYLTGVWVIAHE | |
| CGHQAYCSSEVVNNLIGLVLHSALLVPYHSWRISHRKHHSNTGSCENDEVFVPVTRSVLA | |
| SSWNETLEDSPLYQLYRIVYMLVVGWMPGYLFFNATGPTKYWGKSRSHFNPYSAIYADRE | |
| RWMIVLSDIFLVAMLAVLAALVHTFSENTMVKFYVVPYFIVNAYLVLITYLQHTDTYIPH | |
| FREGEWNWLRGALCTVDRSFGPFLDSVVHRIVDTHVCHHIFSKMPFYHCEEATNAIKPLL | |
| GKFYLKDTTPVPVALWRSYTHCKFVEDDGKVVFYKNKL* | |
| (Codon-optimised pi(w3)-desaturase from | |
| Phytophthora infestans) | |
| SEQ ID NO: 11 | |
| 1 | ATGGCTACAAAGGAGGCTTACGTTTTCCCAACTCTCACCGAGATCAAGAGATCTCTCCCA |
| 61 | AAGGATTGCTTCGAGGCTTCTGTGCCTTTGTCTCTCTACTACACTGTGAGATGCTTGGTT |
| 121 | ATTGCTGTGGCTTTGACCTTCGGATTGAACTACGCTAGAGCTTTGCCAGAGGTTGAGTCT |
| 181 | TTCTGGGCTTTGGATGCTGCTTTGTGCACTGGATATATCCTCCTCCAGGGAATTGTGTTC |
| 241 | TGGGGATTCTTCACTGTTGGACACGATGCTGGACACGGAGCTTTCTCTAGATACCACCTC |
| 301 | TTGAACTTCGTTGTGGGAACCTTCATGCACTCTCTCATCTTGACCCCATTCGAGTCTTGG |
| 361 | AAGTTGACCCACAGACACCACCACAAGAACACCGGAAACATCGATAGAGATGAGGTGTTC |
| 421 | TACCCACAGAGAAAGGCTGATGATCACCCATTGTCCAGGAACTTGATCTTGGCTTTGGGA |
| 481 | GCTGCTTGGCTTGCTTATTTGGTGGAGGGATTCCCACCAAGAAAGGTGAACCACTTCAAC |
| 541 | CCATTCGAGCCACTTTTTGTGAGACAAGTGTCCGCTGTGGTTATCTCTTTGCTCGCTCAC |
| 601 | TTCTTCGTTGCTGGACTCTCTATCTACTTGTCTCTCCAGTTGGGACTTAAGACCATGGCT |
| 661 | ATCTACTACTACGGACCAGTTTTCGTGTTCGGATCTATGTTGGTGATTACCACCTTCTTG |
| 721 | CACCACAACGATGAGGAGACTCCATGGTATGCTGATTCTGAGTGGACTTACGTGAAGGGA |
| 781 | AACTTGTCCTCTGTGGATAGATCTTACGGTGCTCTCATCGATAACCTCTCCCACAACATC |
| 841 | GGAACTCACCAGATCCACCACCTCTTCCCAATTATCCCACACTACAAGCTCAAGAAGGCT |
| 901 | ACTGCTGCTTTCCACCAAGCTTTCCCAGAGCTTGTGAGAAAGTCCGATGAGCCAATCATC |
| 961 | AAGGCTTTCTTCAGAGTGGGAAGGTTGTATGCTAACTACGGAGTGGTTGATCAAGAGGCT |
| 1021 | AAGCTCTTCACTTTGAAGGAGGCTAAGGCTGCTACTGAAGCTGCTGCTAAGACCAAGTCT |
| 1081 | ACCTGA |
| (pi(w3)-desaturase from Phytophthora infestans) | |
| SEQ ID NO: 12 | |
| 1 | MATKEAYVFPTLTEIKRSLPKDCFEASVPLSLYYTVRCLVIAVALTFGLNYARALPEVES |
| 61 | FWALDAALCTGYILLQGIVFWGFFTVGHDAGHGAFSRYHLLNFVVGTFMHSLILTPFESW |
| 121 | KLTHRHHHKNTGNIDRDEVFYPORKADDHPLSRNLILALGAAWLAYLVEGFPPRKVNHEN |
| 181 | PFEPLFVRQVSAVVISLLAHFFVAGLSIYLSLOLGLKTMAIYYYGPVFVFGSMLVITTEL |
| 241 | HHNDEETPWYADSEWTYVKGNLSSVDRSYGALIDNLSHNIGTHQIHHLFPIIPHYKLKKA |
| 301 | TAAFHQAFPELVRKSDEPIIKAFFRVGRLYANYGVVDQEAKLFTLKEAKAATEAAAKTKS |
| 361 | T* |
| (codon-optimised Hpw-3, a w3-desaturase from | |
| Hyaloperonospora parasitica) | |
| SEQ ID NO: 13 | |
| 1 | ATGGCTACTAAACAATCAGTTGCTTTTCCTACTTTGACTGATCTTAAAAGATCTCTTCCT |
| 61 | TCTGAGTGTTTTGAATCTTCTTTGCCTCTTTCTCTTTACTATACACTTAGATCTTTGGTT |
| 121 | TTTGCTGGTTCTCTTGCTGTTTCTCTTTCTTACGCTCTTGCTCAACCTTTGGTTCAAAAC |
| 181 | TTTTACCCTCTTAGAGTTGCTCTTATTGCTGGATACACTGTTTTTCAAGGAGTTATTTTC |
| 241 | TGGGGATTTTTCACTATTGGTCATGATGCTGGTCATGGTGCTTTTTCTAGATATCCTGTT |
| 301 | CTTAACTTCACTGTTGGAACACTTATGCATTCTCTTATTTTGACTCCTTTTGAATCTTGG |
| 361 | AAGTTGACTCATAGACATCATCATAAAAACACTGGAAATATCGATAGAGATGAGATCTTC |
| 421 | TACCCTCAAAGAGAATCTGATGATCATCCTGTTTCTAGACATCTTACTTTCACTCTTGGA |
| 481 | GCTGCTTGGTTCGCTTACCTTGTTGAGGGTTTTCCACCTAGAAAATTGAATCATTACAAT |
| 541 | CCTTTCGAGCCATTGTTCGAGAGAAGAGTTTCTGCTGTTGTTATCTCTATCTTGGCTCAG |
| 601 | TTTTTCGTTGCAGGATTGTCTATTTACTTGTGTTTCCAGGTTGGAGTTCAGGCTGTTGCT |
| 661 | CTTTACTATTACGGTCCTATCTTCGTTTTTGGTACTATGCTTGTTATTACTACTTTTCTT |
| 721 | CATCATAACGATGAAGAGACTCCTTGGTACGGTGATGAGGATTGGTCTTACGTTAAGGGT |
| 781 | AACTTGTCTTCTGTTGATAGATCTTACGGTCCTCTTATCGATAACTTGTCTCATAACATC |
| 841 | GGTACTCATCAAGTTCATCATCTTTTCCCAATCATCCCTCATTACAAATTAAAGCCTGCT |
| 901 | ACAGCTGCTTTCAGAAGAGCTTTCCCACATCTTGTTAGAAAGTCTGATGAAAGAATTTTG |
| 961 | CAGGCTTTTTACAGAATTGGTAGATTGTATGCTAAATATGGTGTTGCTGATTCTTCTGCT |
| 1021 | AAATTGTTTACATTGAAGGAAGCTCAACTTACTTCTAAAGCTGCTTCTGATGCTAAAGCT |
| 1081 | GCTTGA |
| (Hpw-3, a w3-desaturase from Hyaloperonospora | |
| parasitica) | |
| SEQ ID NO: 14 | |
| 1 | MATKQSVAFPTLTDLKRSLPSECFESSLPLSLYYTLRSLVFAGSLAVSLSYALAQPLVON |
| 61 | FYPLRVALIAGYTVFQGVIFWGFFTIGHDAGHGAFSRYPVLNFTVGTLMHSLILTPFESW |
| 121 | KLTHRHHHKNTGNIDRDEIFYPQRESDDHPVSRHLTFTLGAAWFAYLVEGFPPRKLNHYN |
| 181 | PFEPLFERRVSAVVISILAQFFVAGLSIYLCFQVGVQAVALYYYGPIFVFGTMLVITTEL |
| 241 | HHNDEETPWYGDEDWSYVKGNLSSVDRSYGPLIDNLSHNIGTHQVHHLFPIIPHYKLKPA |
| 301 | TAAFRRAFPHLVRKSDERILQAFYRIGRLYAKYGVADSSAKLFTLKEAQLTSKAASDAKA |
| 361 | A- |
| (codon-optimised TpDesK A4-desaturase from | |
| Thalassiosira pseudonana) | |
| SEQ ID NO: 15 | |
| ATGGGTAATGGTAATCTTCCAGCATCTACAGCACAACTCAAGTCAACAAGTAAACCTCAA | |
| CAGCAACACGAGCACAGAACAATCAGTAAATCTGAATTGGCACAACATAACACTCCTAAG | |
| TCTGCTTGGTGTGCAGTTCATTCAACTCCTGCTACAGATCCAAGTCACTCTAATAACAAA | |
| CAGCATGCACACCTTGTTTTGGATATTACAGATTTCGCTTCTAGACATCCAGGAGGAGAT | |
| TTGATTCTTTTGGCTTCAGGAAAAGATGCAAGTGTGCTCTTCGAGACCTACCACCCTAGG | |
| GGAGTTCCAACTTCATTAATTCAAAAGCTTCAGATCGGTGTTATGGAAGAGGAAGCTTTT | |
| AGAGATAGTTTCTACTCTTGGACAGATTCTGATTTCTACACCGTTCTTAAGAGAAGGGTT | |
| GTGGAAAGATTAGAGGAAAGGGGACTTGATAGAAGGGGTTCAAAAGAGATTTGGATCAAG | |
| GCTTTATTTCTCTTAGTTGGATTCTGGTACTGTCTTTACAAGATGTACACTACATCAGAT | |
| ATAGATCAATACGGAATAGCTATTGCATATAGTATCGGAATGGGTACTTTTGCTGCATTC | |
| ATCGGTACATGCATACAACATGATGGAAACCACGGTGCTTTCGCACAGAACAAGCTTTTG | |
| AACAAGTTGGCTGGATGGACACTCGATATGATCGGTGCTTCTGCATTCACCTGGGAATTG | |
| CAGCATATGCTCGGTCATCACCCTTACACTAATGTTCTTGATGGAGTGGAGGAAGAGAGA | |
| AAAGAAAGGGGAGAGGATGTGGCTTTGGAAGAGAAGGATCAAGAGTCAGATCCAGATGTT | |
| TTCTCTTCATTCCCTCTCATGAGAATGCATCCACATCACACCACTAGTTGGTACCATAAA | |
| TATCAGCACCTTTATGCTCCTCCACTCTTTGCATTAATGACCCTTGCTAAGGTGTTTCAA | |
| CAGGATTTCGAAGTTGCAACATCTGGAAGATTGTACCATATTGATGCTAACGTTAGATAT | |
| GGTTCAGTTTGGAATGTGATGAGATTCTGGGCTATGAAAGTTATCACAATGGGATACATG | |
| ATGGGTTTGCCTATTTACTTTCATGGAGTTCTCAGGGGAGTGGGTCTTTTCGTTATCGGA | |
| CACCTTGCATGTGGTGAACTCTTAGCTACTATGTTCATAGTTAACCATGTGATTGAGGGA | |
| GTGAGTTATGGTACAAAAGATCTTGTTGGAGGTGCATCTCACGGAGATGAAAAGAAAATT | |
| GTGAAGCCTACAACCGTTTTAGGTGATACCCCAATGGAGAAAACTAGAGAAGAGGCTCTC | |
| AAGTCAAACAGTAACAACAACAAGAAAAAGGGAGAAAAGAACTCAGTTCCTAGTGTGCCA | |
| TTTAATGATTGGGCTGCAGTGCAATGCCAGACTTCTGTTAACTGGTCTCCTGGTTCATGG | |
| TTTTGGAATCATTTCAGTGGAGGTTTGTCTCACCAAATCGAGCATCACCTCTTCCCAAGT | |
| ATATGTCATACTAACTACTGCCACATTCAAGATGTTGTGGAATCTACATGTGCTGAGTAC | |
| GGTGTGCCATATCAGTCTGAATCAAACTTGTTCGTTGCATACGGAAAGATGATCTCACAT | |
| TTGAAGTTCCTCGGTAAGGCTAAGTGCGAGTGA | |
| (TpDesK Δ4-desaturase from Thalassiosira | |
| pseudonana) | |
| SEQ ID NO: 16 | |
| 1 | MGNGNLPASTAQLKSTSKPQQQHEHRTISKSELAQHNTPKSAWCAVHSTPATDPSHSNNK |
| 61 | QHAHLVLDITDFASRHPGGDLILLASGKDASVLFETYHPRGVPTSLIQKLQIGVMEEEAF |
| 121 | RDSFYSWTDSDFYTVLKRRVVERLEERGLDRRGSKEIWIKALFLLVGFWYCLYKMYTTSD |
| 181 | IDQYGIAIAYSIGMGTFAAFIGTCIQHDGNHGAFAQNKLLNKLAGWTLDMIGASAFTWEL |
| 241 | QHMLGHHPYTNVLDGVEEERKERGEDVALEEKDOESDPDVFSSFPLMRMHPHHTTSWYHK |
| 301 | YQHLYAPPLFALMTLAKVFQQDFEVATSGRLYHIDANVRYGSVWNVMRFWAMKVITMGYM |
| (O809Δ4 Δ4-desaturase from Ostreococcus RCC809) | |
| SEQ ID NO: 17 | |
| atgccgacgactcgatcgcgcgcgcgcgtgacgacgccccctcgcgagacgccgacgaga | |
| gcgaacaccgtcgccgcgctcgatcccgagcgcaagtacacgcgcattcgcggcgtcgtg | |
| tacgacgtcacggatttcgccagccgtcatccgggtggcgcgcaattgttatcgctgtgc | |
| gtggggagagacgccaccatcctggtggagagtcatcaccttcgtccggaggtggtgcaa | |
| aagtacctgaagacgcttcccgtggtggagggcgcggcgggggcgttcgggcccgaggag | |
| acgtttccgaaaccgctcgactcggatttgtaccgaaagattcaggggcgcgttcgtaaa | |
| gagatcgtcgaaccgttgaagatgacgcgcggacgcgagccgcacgggcgaggctggtgc | |
| gtgttggacgccggggtggtgttggctttcttcgcgttcgcgttgggagtctattggaag | |
| acgccgacggtggcgacggggtgcctgttggggctcgccgggtactggagcggcaccgga | |
| ttgcaacacacggcgaaccacggtggattggcgaagagtgggttttggaatcagttttgg | |
| ggatggctcgggaacgacgtcgccatcgggaagagctcggtggagtggagatatcatcac | |
| atggtgagccaccactcgtattgcaacgacgcggacctcgatcaagacgtgtacaccgcg | |
| ctgccgcttcttcgtttggacccgtcccaggagttgaagtggttccaccgctaccaagcg | |
| ttctacgcgccgctgatgtggccgatgttgtggctcgccgcgcagtttggcgacgcgcaa | |
| aatattttagtggataaggcgtctccgggcgtcgagtacaagggcctcatgaagctcgaa | |
| gtcgcgctgtacgttctcggaaagtttttgcattttagcttgttgctcggcgtaccggcc | |
| tacttgcacgggtttgcgaacgccatcgtgccgttcatcgcgtacggtgcgttcggttcg | |
| ttcgtcctgtgctggtttttcatcgtcagtcacaacttggaggcgttgaccccaatcaat | |
| ctgagcaaatccacgaagaatgactggggcgcgtggcaaatcgaaacttccgcgtcctgg | |
| ggcaacggcttctggagctttttctccggcgggttgaatttgcaaatcgagcaccacttg | |
| ttcccgggttgcgcgcacaacttgtacccgaagatggttcccatcatcaaggaagagtgc | |
| gaaaaggctggcgtcacgtacaccggttacggtgggtactttggtctccttcccatcact | |
| cgggacatgttcgcgtacttgtacaaaatgggccgacaaagcaaaaagtcggcgtaa | |
| (O809Δ4 Δ4-desaturase from Ostreococcus RCC809) | |
| SEQ ID NO: 18 | |
| MPTTRSRARVTTPPRETPTRANTVAALDPERKYTRIRGVVYDVTDFASRHPGGAQLLSLC | |
| VGRDATILVESHHLRPEVVQKYLKTLPVVEGAAGAFGPEETFPKPLDSDLYRKIQGRVRK | |
| EIVEPLKMTRGREPHGRGWCVLDAGVVLAFFAFALGVYWKTPTVATGCLLGLAGYWSGTG | |
| LQHTANHGGLAKSGFWNQFWGWLGNDVAIGKSSVEWRYHHMVSHHSYCNDADLDQDVYTA | |
| LPLLRLDPSQELKWFHRYQAFYAPLMWPMLWLAAQFGDAQNILVDKASPGVEYKGLMKLE | |
| VALYVLGKFLHESLLLGVPAYLHGFANAIVPFIAYGAFGSFVLCWFFIVSHNLEALTPIN | |
| LSKSTKNDWGAWQIETSASWGNGEWSFFSGGLNLQIEHHLFPGCAHNLYPKMVPIIKEEC | |
| EKAGVTYTGYGGYFGLLPITRDMFAYLYKMGRQSKKSA | |
| (PerfD15 Δ15-desaturase from Perilla frutescens). | |
| During gene synthesis, CDS codon optimized to | |
| Arabidopsis. Also, add CC in front of ATG to form | |
| Ncol CCATGG, and add Pacl TTAATTAA following stop | |
| codon TGA. | |
| SEQ ID NO: 19 | |
| CCATGGCTGTTTCTTCTGGAGCTAGGCTTTCTAAGTCTGGAGCTGATGGAGAAGTTTTCG | |
| ATGGACAGCAACAGTACGAGGGAATTGGAAAGAGAGCTGCTGATAAGTTTGATCCTGCTG | |
| CTCCTCCTCCTTTCAAGATCGCTGATATCAGGGCTGCTATTCCTGCTCATTGCTGGGTTA | |
| AGAACCCTTGGAGGAGTCTTTCTTACGTTGTGTGGGATGTTGCTGCTGTTTTCGCTCTTC | |
| TTGCTGCTGCTGTGTACATTAACTCTTGGGCTTTCTGGCCTGTTTACTGGATTGCTCAGG | |
| GAACTATGTTCTGGGCTCTTTTCGTTCTTGGACACGATTGTGGACACGGATCTTTCTCTG | |
| ATAACACTACTCTTAACAACGTTGTGGGACACGTTCTTCACTCTTCTATCCTTGTGCCTT | |
| ACCACGGATGGAGAATCTCTCATAGGACTCACCATCAGAACCATGGACACGTTGAGAAGG | |
| ATGAGTCTTGGGTTCCACTTCCTGAGAACCTTTACAAGAAGCTTGATTTCTCTACTAAGT | |
| TCCTTAGGTACAAGATCCCTTTCCCTATGTTCGCTTACCCTCTTTACCTTTGGTACAGAT | |
| CTCCTGGAAAGACTGGATCTCACTTCAACCCTTACTCTGATCTTTTCAAGCCTAACGAGA | |
| GGGGACTTATCGTGACTTCTACTATGTGTTGGGCTGCTATGGGAGTGTTTCTTCTTTACG | |
| CTTCTACTATCGTGGGTCCTAACATGATGTTCAAGCTTTACGGAGTGCCTTACCTTATTT | |
| TCGTGATGTGGCTTGATACTGTGACTTACCTTCACCACCACGGATACGATAAGAAGCTTC | |
| CTTGGTACAGGTCAAAGGAGTGGTCTTACCTTAGAGGAGGACTTACTACTGTGGATCAGG | |
| ATTACGGATTCTTCAACAAGATCCACCACGATATTGGAACTCACGTGATCCATCACCTTT | |
| TCCCTCAGATTCCTCACTACCACCTTGTTGAGGCTACTAGAGAGGCTAAGAGGGTGTTGG | |
| GAAACTACTACCGTGAGCCTAGAAAGTCTGGACCTGTGCCTCTTCATCTTATCCCTGCTC | |
| TTTTGAAGTCTCTTGGAAGGGATCACTACGTGTCTGATAACGGAGATATCGTGTACTACC | |
| AGACTGATGATGAGCTTTTCCCTTCTAAGAAGATCTGATTAATTAA | |
| (PerfD15 Δ15-desaturase from Perilla frutescens). | |
| SEQ ID NO: 20 | |
| MAVSSGARLSKSGADGEVFDGQQQYEGIGKRAADKFDPAAPPPFKIADIRAAIPAHCWVK | |
| NPWRSLSYVVWDVAAVFALLAAAVYINSWAFWPVYWIAQGTMFWALFVLGHDCGHGSFSD | |
| NTTLNNVVGHVLHSSILVPYHGWRISHRTHHQNHGHVEKDESWVPLPENLYKKLDFSTKF | |
| LRYKIPFPMFAYPLYLWYRSPGKTGSHFNPYSDLFKPNERGLIVTSTMCWAAMGVFLLYA | |
| STIVGPNMMFKLYGVPYLIFVMWLDTVTYLHHHGYDKKLPWYRSKEWSYLRGGLTTVDQD | |
| YGFFNKIHHDIGTHVIHHLFPQIPHYHLVEATREAKRVLGNYYREPRKSGPVPLHLIPAL | |
| LKSLGRDHYVSDNGDIVYYQTDDELFPSKKI | |
| (MsqΔ6 Δ6-desaturase derived from Mantoniella | |
| squamata) During gene synthesis, CDS codon | |
| optimized to Arabidopsis. Also, add CC in front | |
| of ATG to form Ncol CCATGG, and add Pacl TTAATTAA | |
| following stop codon TAA. | |
| SEQ ID NO: 21 | |
| CCATGGGACCTCCTAAGGAATCTACTCGTAAGAACGCTGGCGGACCTCTCACCCGTGGCA | |
| AGCTCTCTGCTGACCTCGCTAAGCTCGAACCTCACAAGCTCGCTCAGACATTCGACACTC | |
| GTTGGGTGAGGGTTGGAGACGTTGAGTACGACGTCACCAACTTCAAGCATCCTGGTGGAT | |
| CCGTGATCTTCTACATGCTCAGTAACACTGGTGCTGACGCCACCGAGGCTTTCAACGAGT | |
| TCCACATGCGTTCCCCTAAGGCTTGGAAGATGCTCAAGGCCCTCCCTAACCGTCCTGCTG | |
| AGACACCTAGGAGTCAGGACCCTGACGGTCCTATGCTCGAAGACTTCGCTAAGTGGCGTG | |
| CCCAGCTCGAAAAGGAAGGATTCTTCAAGCCTTCTATCGCTCACGTCGCCTACAGGATCG | |
| CCGAACTCGCTGCCATGTTCGCTCTCGGCTGCTACATCATGTCTCTCGGTTACCCTGTCG | |
| TGGCTTCCATCGTGTTCGGCGCTTTCTTCGGTGCCCGTTGCGGATGGGTCCAGCACGAAG | |
| GCGGTCATAACTCTCTCACCGGAAACATCTGGCTCGACAAGCGTATCCAGGCTGCCACCT | |
| GCGGTTTCGGACTCTCTACATCCGGCGACATGTGGAACCAGATGCACAACAAGCACCATG | |
| CTACACCTCAGAAGGTGCGTCATGACATGGACCTCGACACCACACCTGCTGTTGCCTTCT | |
| TCAAGACTGCCGTCGAGGACAACCGTCCTAGGGGCTTCTCCCGTGCTTGGAGTAGGGCTC | |
| AGGCCTGGACATTCGTCCCTGTGACTTCTGGTCTCCTCGTCCAGATGTTCTGGATCTACG | |
| TCCTCCACCCTCGTCAGGTGGCCAGGAAGAAGAACTACGAGGAGGCTTCTTGGATGATCC | |
| TCTCCCATGTGCTCCGTACTGCTACCATCAAGTACGCCGGAGGCTACAGTTGGCCTGTCG | |
| CCTACCTCTGGTTCTCTTTCGGTAACTGGATCGCCTACATGTACCTCTTCGCTCACTTCA | |
| GTACCTCTCACACACACCTCGAAGTTGTCCCTAGTGACAAGCACATCTCTTGGGTGAACT | |
| ACGCTGTTGACCATACCGTCGACATCGACCCTTCTAAGGGATACGTGAACTGGCTCATGG | |
| GCTACCTCAACTGCCAGGTTATCCACCATCTCTTCCCTGACATGCCTCAGTTCCGTCAGC | |
| CTGAAGTGTCCCGTAGGTTCGTTGCTTTCGCCAAGAAGTGGAACCTCAACTACAAGGTCC | |
| TCACTTACTACGGTGCTTGGAAGGCCACCTTCACAAACCTCGACACAGTTGGTCAGCACT | |
| ACTACAAGCACGGAAAGGCCCACGCCCACTAATTAATTAA | |
| (MsqΔ6 Δ6-desaturase derived from | |
| Mantoniella squamata) | |
| SEQ ID NO: 22 | |
| MGPPKESTRKNAGGPLTRGKLSADLAKLEPHKLAQTFDTRWVRVGDVEYDVTNFKHPGGS | |
| VIFYMLSNTGADATEAFNEFHMRSPKAWKMLKALPNRPAETPRSQDPDGPMLEDFAKWRA | |
| QLEKEGFFKPSIAHVAYRIAELAAMFALGCYIMSLGYPVVASIVFGAFFGARCGWVQHEG | |
| GHNSLTGNIWLDKRIQAATCGFGLSTSGDMWNQMHNKHHATPQKVRHDMDLDTTPAVAFF | |
| KTAVEDNRPRGFSRAWSRAQAWTFVPVTSGLLVQMFWIYVLHPRQVARKKNYEEASWMIL | |
| SHVLRTATIKYAGGYSWPVAYLWFSFGNWIAYMYLFAHFSTSHTHLEVVPSDKHISWVNY | |
| AVDHTVDIDPSKGYVNWLMGYLNCQVIHHLFPDMPQFRQPEVSRRFVAFAKKWNLNYKVL | |
| TYYGAWKATFTNLDTVGQHYYKHGKAHAH | |
| (codon optimised O809D6 Δ6-desaturase from | |
| Ostreococcus RCC809) | |
| SEQ ID NO: 23 | |
| ATGGGAAAGGGAGCAAGGAACCCAGGAGCAAGGGCATGGAAGTCAACATTGGAGCCTCAC | |
| GCAGTGGCAAAGTCATTCGATAGGAGATGGGTTAAGGTGGATGGAGTTGAATACGATGTG | |
| ACTGATTTCAAGCATCCTGGAGGTAGTGTTATATACTACATGCTTTCTAACACAGGTGCT | |
| GATGCAACCGAAGCTTTTAAGGAGTTCCATTACAGGAGTAAGAAAGCTAGGAAAGCACTT | |
| GCTGCATTGCCTCAAAGAGAACCAGAGGATGCTTCACCAGTTGAAGATGCAAACATGCTC | |
| AAGGATTTCGCTAAGTGGAGAAAGGATCTCGAAAGGGAGGGATTTTTCAAACCTTCTCCA | |
| GCTCATGTGGCATATAGATTTGCTGAGCTTGCTGCAATGTTCGCTCTCGGTACAGCATTA | |
| ATGTACGCTAGATGGCACGCAACTTCTGTTTTCGTGACAGCTTGTTTCTTTGGAGCAAGA | |
| TGCGGTTGGGTTCAACATGAGGGAGGTCACTCTTCATTGACTGGATCAATCTGGTGGGAT | |
| AAGAGAATACAGGCTTTTACAGCAGGATTCGGTCTCGCTAGTTCTGGTGATATGTGGAAT | |
| TTAATGCATAACAAGCATCACGCAACCCCTCAAAAAGTTAGGCACGATATGGATTTGGAT | |
| ACTACACCAGCTGTTGCATTTTTCAATACTGCTGTGGAAGAGAACAGACCTAGGAAGTTT | |
| TCTAAACTTTGGTTGAGAGTTCAGGCTTGGACCTTCGTTCCTGTGACTTCAGGACTCGTG | |
| CTTTTGGCTTGGATGTATCTCTTACATCCAAGACACATTGCAAGAAGGAAGAATTACGAA | |
| GAGGCTGCATGGATCGTTGCTGCACATGTGATAAGGACATCAGTTATTAAAGCTGTGACA | |
| GGATATAGTTGGATAACCTGTTACGGTCTCTTTTTAAGTACCATGTGGGTTTCTGGATGC | |
| TATCTTTTTGCTCATTTCTCAACCAGTCATACTCACCTTGATGTTGTGCCTTCAGATAAG | |
| CATTTGAGTTGGGTTAGATATGCTGTGGATCACACTATTGATATCGATCCATCTAAATCA | |
| GTTGTGAATTGGCTTATGGGTTACTTGAACTGTCAGGTTATCCATCACTTGTTTCCTGAT | |
| ATGCCACAATTCAGACAGCCAGAAGTTTCTAGAAGGTTTGTGTCATTCGCTAAGAAATGG | |
| AATCTCAACTACAAGGTTATGTCTTATTACGGAGCTTGGAAAGCAACATTCGGTAACCTT | |
| AACGAAGTTGGAAAGCACTACTATATTCAGGGTTCTCAAATCACAAAAAAGACCGTGTAA | |
| (O809D6 Δ6-desaturase from Ostreococcus RCC809) | |
| SEQ ID NO: 24 | |
| 1 | MGKGARNPGARAWKSTLEPHAVAKSFDRRWVKVDGVEYDVTDFKHPGGSVIYYMLSNTGA |
| 61 | DATEAFKEFHYRSKKARKALAALPQREPEDASPVEDANMLKDFAKWRKDLEREGFFKPSP |
| 121 | AHVAYRFAELAAMFALGTALMYARWHATSVFVTACFFGARCGWVQHEGGHSSLTGSIWWD |
| 181 | KRIQAFTAGFGLASSGDMWNLMHNKHHATPQKVRHDMDLDTTPAVAFFNTAVEENRPRKF |
| 241 | SKLWLRVQAWTFVPVTSGLVLLAWMYLLHPRHIARRKNYEEAAWIVAAHVIRTSVIKAVT |
| 301 | GYSWITCYGLFLSTMWVSGCYLFAHFSTSHTHLDVVPSDKHLSWVRYAVDHTIDIDPSKS |
| 361 | VVNWLMGYLNCQVIHHLFPDMPQFRQPEVSRRFVSFAKKWNLNYKVMSYYGAWKATFGNL |
| 421 | NEVGKHYYIQGSQITKKTV- |

Δ4-desaturase Ostreococcus RCC809 nucleic acid
codon optimised acid for expression in Pt
SEQ ID No. 25
ggatccggtaccaagcttgatatcaccaaaatgccaactactcgttctcgtgctcgtgtt
actactccacctcgtgaaactcctactcgtgctaatactgttgctgctttagatccagaa
cgtaaatatacacgtattcgaggtgttgtatatgatgttactgattttgctagtcgacat
ccaggtggtgcacaattattatctttatgtgttggtcgtgatgctacaattttagtagaa
tcacatcatttacgaccagaagttgtacaaaaatatttaaaaacattacctgttgtagaa
ggtgctgctggtgcatttggtccagaagaaacttttccaaaacctttagatagtgattta
tatcgtaaaattcaaggtcgtgttcgaaaagaaattgtagaaccattaaaaatgacacgt
ggtcgagaacctcatggtcgtggttggtgtgttttagatgctggtgttgtattagctttc
tttgcttttgcattaggtgtttattggaaaacaccaactgtagctactggttgtttatta
ggtttagcaggttattggtctggtacaggtttacaacatactgctaatcatggtggttta
gcaaaatcaggttttggaatcaattttggggttggttaggaaatgatgttgctattggta
aatcaagtgtagaatggcgttatcatcatatggtttcacatcatagttattgtaatgatg
ctgatttagatcaagatgtttatacagcattaccattattacgtttagatccttcacaag
aattaaaatggtttcatcgttatcaagcattttatgcacctttaatgtggcctatgttat
ggttagctgcacaatttggtgatgctcaaaatattttagttgataaagcaagtccaggtg
tagaatataaaggtttaatgaaattagaagttgctttatatgtattaggaaaatttttac
atttttctttattattaggtgttcctgcatatttacatggttttgctaatgcaattgtac
catttattgcttatggtgcatttggttcatttgttttatgttggtttttcattgtaagtc
ataatttagaagcattaacaccaattaatttatctaaatcaactaaaaatgattggggtg
cttggcaaattgaaactagtgcatcttggggtaatggtttttggtcatttttctcaggtg
gtttaaatttacaaattgaacatcatttatttcctggttgtgctcataatttatatccaa
aaatggttcctattattaaagaagaatgtgaaaaagcaggtgttacatatactggttatg
gtggttattttggtttattaccaattactcgtgatatgtttgcttatttatataaaatgg
gtcgtcaatctaaaaaatctgcttaagagctcggtaccctcgagtctaga
FAE1-A(Csa11g007400)
SEQ ID NO: 28
ATTTCGAAATATTTGGCCTTAACTAAACTTCCACCATGATTTATTTACTGATCTAGTTCG
GGGACAGACTTTGCGAATAAAACTCATTACCGAGAAACATTCATCCCATAATTGCTATTT
AGTCAGAGGCTAATCGACTATGGCCTTTCAGCCAATCAAAGCTACGAACACGAATCTCCC
TAAAACATCCTCAAGTATTTTATTTAATACACATGTATCGTATTGAGCACCACTCATAAA
CTAATTTCATACATTTATCATACTCTTTATTTGTAATAATAAAAGCATCAACATATTGTA
GGCAATTAGAATCAAAACAAAACATTTTTTTTTTCTTTCCAAATTTTCAAAATTGGTAAA
CGAAACTTGGACCTTTAATACTTATATTGGCAATATAATAATATTGCAGAGTGGACTATT
TCCCTTATTTTGGCAACTTTCAGTGGACTAGTAATTTATTTCAATGTGGATGCTTGCATG
AGTGTGAATATACACATGTCTATATGCATGCCTGCAAATCGTAACGGACCACAAAAAAGG
ATCCATACAAATACCTCTTAACGGCTCCTCTCTATCATACTCTCCGACACAAACTGAGCA
ATGACGTCCGTTAACGCAAAGCTCCTTTACCATTACGTCCTAACCAACTTTTTCAACCTT
TGCTTGTTTCCGTTAACGGCGTTACTTGCCGGAAAAGCCTCTAGGCTTACCTCAAACGAT
CTCTACCACTTCTATTCCCATCTCCAACACAACCTTATAACCGTAATTTTACTCTTTGCT
TTCACCGCTTTCGGTTTGGTTCTCTACATTGTAACCCGGCCCAAACCGGTTTACCTCGTT
GACTACTCGTGCTACCTTCCACCACCGCATCTCAAAGTTAGTGTTTCCAAGGCGATGGAT
ATTTTCTACCAAATAAGAAAAGCTGATACCTCACGGAACGTGGCATGCGATGATCCATCC
TCGCTTGATTTCCTGAGGAAGATTCAAGAACGTTCAGGTCTAGGTGATGAAACCTACAGT
CCCCAGGGACTCATTAACGTGCCCCCACGAAAGACCTTTGCAGCTTCACGTGAAGAGACA
GAGCAGGTAATCATCGGTGCGCTAGATAAGCTATTCGAGAATACCAAAGTTAACCCTAGA
GAAATTGGTATACTTGTGGTCAACTCAAGCATGTTTAATCCAACTCCTTCGCTATCTGCG
ATGGTCGTTAATACTTTCAAGCTTCGAAGCAACATCAAAAGCTTTAGTCTCGGAGGAATG
GGTTGTAGTGCTGGTGTCATCGCCATTGATCTTGCAAAGGACTTGTTGCATGTTCATAAA
AACACTTATGCACTTGTGGTGAGCACTGAGAACATCACTCAAGGCATTTATGCTGGCGAA
AATAGATCCATGATGGTTAGCAATTGCTTGTTCCGTGTTGGTGGCGCAGCGATTTTGCTC
TCCAACAAGCCAGGAGATCGGAGACGGTCCAAGTACAAGTTATGTCATACTGTTCGAACG
CATACCGGAGCTGATGACATGTCTTTTCGATGTGTGCAACAAGGAGACGATGAGAGCGGT
AAAATCGGAGTTTGTCTGTCAAAGGACATAACCGTTGTTGCGGGGATAGCGCTTAAGAAA
AACATAGCAACGTTGGGTCCGTTGATTCTTCCTTTAAGCGAAAAATTTCTGTTTTTAGTA
ACCTTCATCGCCAAGAAACTTTTGAAGGACAAGATCAAGCACTATTACGTCCCGGATTTC
AAGCTTGCTATTGACCATTTCTGTATTCATGCGGGAGGCAGAGCCGTGATCGATGTGCTT
GAGAAGAGCTTAGGACTATCTCCAATCGATGTGGAGGCATCTAGATCAACGTTACACAGA
TTTGGGAATACTTCGTCTAGCTCAATTTGGTATGAATTGGCATACATAGAAGCAAAAGGA
AGGATGAAGAAAGGGAATAGAGCTTGGCAGATTGCTTTAGGGTCAGGATTTAAGTGTAAC
AGTGCGGTTTGGGTGGCTCTATGCAATGTCAAGGCTTCGGCGAATAGTCCTTGGGAACAT
TGCATCGATAGATATCCGGTTCAAATTGATTCTGGTTCATCAAAATCAGATACTCATGTC
AAAAACGGTCGGACCTAATTCATCTATCTGAATCTGAATGTTAACCATTTAGTGTTTTTT
TTTTTCCTTTCCTTCTTTCTTTTATTGGTTAAGATTTGATAATGTTTATGATGTTCTCTC
GTTTTCGTTATAAATAAAGAAACACATGGGTGTTTCCATTCTTCTATTTAAGTTGTGGAC
CGTTTAATAACCTAACTTAGAATTCAATCATCTTTTTTTGTTCTTCTGTTTTTAAATCTT
CCAACCAATCAACGTGTTTTCATACTAACATAATTATTAAAACGTGTTTAAAATTAAAAA
TCTCTTTGGTGTTTCTAGAGGTTTTAGATTGTTTGCTCTGTTTGCCACCTGTTTTTTTTT
CCCCCCGTCAGCTCCTGTATTTGAGATTTAGATATATAAATGCATATNAAACTAAAAAAA
ACTAGAAAAAGAAATAAAGAAAGCAATATACCTCTGCTTGTGATATGGAAGTGGAAGAAA
TACAAGATTTGATCGATTCTCATCATCTTGAGATGACTGAATAGTCGGAACCATGGAAAC
ACCAAACACACAGGATCATGACTGGTTCTCTACTCTCACTCGTCCTCGTTCCTGCATTCT
CGCATCCTTGAGCCGTCGTTGATATATCCACCGTGTGAAAACGTTTCGGTGATCGTAGAG
AAAATTTTGACCTAATTTAAATCGATGACGATCGGATGAAGATGATGTAGTCACGATTTA
ACAAGAGAGATTAATCACGAAAAGAGATGCTCTCCGCTCCGATCAAATATCAAAAAGAGA
TCATCTCTTTTTGTGATTGTGTAAGAAGTTAGAGAGAAGACTTGACGTCCTGTAATCGTG
ATCTCGATCGAATGAAGATATGTCGTCACGATTAACATGAGGATGGCGATCAATCAATCA
CGAACAGAGTCAGAGATGCTTCCCCTAATCTTGACAGAGATCACCAACGCAAAGCTTTGG
ATTTGGTTTCTCAAAGAAGAAGAAGAAGAGAGATTTGGAAAACTTTGACAAAGAAGAAAA
TGAGAGAATGAATGAATGATGTGATGTTGGAGATTTTTTAACCTAATGACTAAATGAGCC
GTCTTTTATATACGCAACAGCTATATTAATATTTTTTTATTTTTTATTTTCTAATTTCAG
CAAATTTAAATAACGAAATTGTGATTGTTTCCTTTGTTTTCTTTTCCCCACGAGTTCTCA
AGTTGTTTGTTTTCTTATTTTGGTTGCAACCAAAAAAAACAAGACAGAAAAAACAATTTA
TCTCAAGAGTAACAAAAAGGAGATTCGAGATCCTTTGGAGTTACTCAATTACATTCATAT
GTCACGAGATAAAAAGGTTAAACAATCACTGGATCAGTGTGCTCATGGTGTTCCAGGTCC
AAAACATGTGTCACGAGATAAAAACAAAGAGAAACACAACTGAAAATGATTACCAAGATC
ACAACTATATATAAAGGACTTATAAAAAATGAATTTGAAAGTGGTTAAACTAAGTGATTA
TAAGTGTTATTGCAGTTACCCCCTTATAGGTTTGGTGAATCTTATTAGAGATAACTTATT
CTTAAGATAGTTGCAATTAACCAAAAAAAAAAATTGTCCGGATAGTTTGATGCAATTAAA
TGATTAATGAGTGTTCTATAGGGTCTGATTCTTAATATTTCGAAATATTTGGCCTTAACT
AAACTTCCACCATGATTTATTTACTGATCTAGTTCGGGGACAGACTTTGCGAATAAAACT
CATTACCGAGAAACATTCATCCCATAATTGCTATTTAGTCAGAGGCTAATCGACTATGGC
CTTTCAGCCAATCAAAGCTACGAACACGAATCTCCCTAAAACATCCTCAAGTATTTTATT
TAATACACATGTATCGTATTGAGCACCACTCATAAACTAATTTCATACATTTATCATACT
CTTTATTTGTAATAATAAAAGCATCAACATATTGTAGGCAATTAGAATCAAAACAAAACA
TTTTTTTTTTCTTTCCAAATTTTCAAAATTGGTAAACGAAACTTGGACCTTTAATACTTA
TATTGGCAATATAATAATATTGCAGAGTGGACTATTTCCCTTATTTTGGCAACTTTCAGT
GGACTAGTAATTTATTTCAATGTGGATGCTTGCATGAGTGTGAATATACACATGTCTATA
TGCATGCCTGCAAATCGTAACGGACCACAAAAAAGGATCCATACAAATACCTCTTAACGG
CTCCTCTCTATCATACTCTCCGACACAAACTGAGCAATGACGTCCGTTAACGCAAAGCTC
CTTTACCATTACGTTCTAACCAACTTTTTCAACCTTTGCTTGTTTCCGTTAACGGCGTTA
CTTGCCGGAAAAGCCTCTAGGCTTACCTCAAACGATCTCTACCACTTCTATTCCCATCTC
CAACACAACCTTATAACCGTAATTTTACTCTTTGCTTTCACCGCTTTCGGTTTGGTTCTC
TACATTGTAACCCGGCCCAAACCGGTTTACCTCGTTGACTACTCGTGCTACCTTCCACCA
CCGCATCTCAAAGTTAGTGTTTCCAAGGCGATGGATATTTTCTACCAAATAAGAAAAGCT
GATACCTCACGGAACGTGGCATGCGATGATCCATCCTCGCTTGATTTCCTGAGGAAGATT
CAAGAACGTTCAGGTCTAGGTGATGAAACCTACAGTCCCCAGGGACTCATTAACGTGCCC
CCACGAAAGACCTTTGCAGCTTCACGTGAAGAGACAGAGCAGGTAATCATCGGTGCGCTA
GATAAGCTATTCGAGAATACCAAAGTTAACCCTAGAGAAATTGGTATACTTGTGGTCAAC
TCAAGCATGTTTAATCCAACTCCTTCGCTATCTGCGATGGTCGTTAATACTTTCAAGCTT
CGAAGCAACATCAAAAGCTTTAGTCTCGGAGGAATGGGTTGTAGTGCTGGTGTCATCGCC
ATTGATCTTGCAAAGGACTTGTTGCATGTTCATAAAAACACTTATGCACTTGTGGTGAGC
ACTGAGAACATCACTCAAGGCATTTATGCTGGCGAAAATAGATCCATGATGGTTAGCAAT
TGCTTGTTCCGTGTTGGTGGCGCAGCGATTTTGCTCTCCAACAAGCCAGGAGATCGGAGA
CGGTCCAAGTACAAGTTATGTCATACTGTTCGAACGCATACCGGAGCTGATGACATGTCT
TTTCGATGTGTGCAACAAGGAGACGATGAGAGCGGTAAAATCGGAGTTTGTCTGTCAAAG
GACATAACCGTTGTTGCGGGGATAGCGCTTAAGAAAAACATAGCAACGTTGGGTCCGTTG
ATTCTTCCTTTAAGCGAAAAATTTCTGTTTTTAGTAACCTTCATCGCCAAGAAACTTTTG
AAGGACAAGATCAAGCACTATTACGTCCCGGATTTCAAGCTTGCTATTGACCATTTCTGT
ATTCATGCGGGAGGCAGAGCCGTGATCGATGTGCTTGAGAAGAGCTTAGGACTATCTCCA
ATCGATGTGGAGGCATCTAGATCAACGTTACACAGATTTGGGAATACTTCGTCTAGCTCA
ATTTGGTATGAATTGGCATACATAGAAGCAAAAGGAAGGATGAAGAAAGGGAATAGAGCT
TGGCAGATTGCTTTAGGGTCAGGATTTAAGTGTAACAGTGCGGTTTGGGTGGCTCTATGC
AATGTCAAGGCTTCGGCGAATAGTCCTTGGGAACATTGCATCGATAGATATCCGGTTCAA
ATTGATTCTGGTTCATCAAAATCAGATACTCATGTCAAAAACGGTCGGACCTAATTCATC
TATCTGAATCTGAATGTTAACCATTTAGTGTTTTTTTTTTTCCTTTCCTTCTTTCTTTTA
TTGGTTAAGATTTGATAATGTTTATGATGTTCTCTCGTTTTCGTTATAAATAAAGAAACA
CATGGGTGTTTCCATTCTTCTATTTAAGTTGTGGACCGTTTAATAACCTAACTTAGAATT
CAATCATCTTTTTTTGTTCTTCTGTTTTTAAATCTTCCAACCAATCAACGTGTTTTCATA
CTAACATAATTATTAAAACGTGTTTAAAATTAAAAATCTCTTTGGTGTTTCTAGAGGTTT
TAGATTGTTTGCTCTGTTTGCCACCTGTTTTTTTTTCCCCCCGTCAGCTCCTGTATTTGA
GATTTAGATATATAAATGCATATTTGAATTGTTTAGGTATTAAAAAAAAAAAAAAAAAAA
AGCAAGGAAGCCCTAAGCTTCCTTTTAAATTAGGTGTGTTTTTTTACTTTAACTTATGAC
GCATTTTTTTGCAACATGTAAATTATTTTGGTATTATTATAGCTAATACTAATAACTATG
ACAGTAATTAAATTATAAGATATATATTTGCATAGAAAATAGAAATATATAAAC
FAE1-B(Csa10g007610)
>10 dna: chromosome chromosome:Cs:10:
2763759:2766674:−1
SEQ ID NO: 29
ATTTGGCCTTAACTAAACTTCCACCTGATTTATTTACTGATCTAGTTCGGGGACAGACTT
TGCGAATAAAACTCGTTCCCGAGAAACATTCATCCCATAACTGCTATTTAGTCAGAGGCT
AATCGACTATGGCCTTTCAGCCAATCAAATCTACGAACACGAATCCCCCTAAAACATCCT
CAAGTATTTATTTAATACACATGTATCGTATTGAGCACCACTCATAAACTATTTTTTTTT
TGTTTTTAACAAAAAAAATTTATCATACTCTTTTGTAATAATAGATGCATCAACATATTG
TAGGCAACGTTGAAGAACCAGTACATTCTTTTTTTTTTTGCTCCAAATTTTCAAAATTGG
AAAATGAAACTTGGACGAAATAAATTTAACACTCTGTATATATTGGCAATATAATATTGC
AGAGTGGACTATTTACCTTATTTTGGCAACTTTCAGTGGACTAGTAATTTATTTCAATGT
GTATGCTTGCATGAGTGTGAATATACACATGTCTATATGCATGCTTGCAAATCGTAACGG
ACCACAAAAAAGGATCCATACAAATACCTCTTAACGGCTCCTCTCTATCATACTCTCCGA
CACAAACTGAGCAATGACGTCCGTTAACGCAAAGCTCCTTTACCATTACGTTCTAACCAA
CTTTTTCAACCTTTGCTTGTTTCCGTTAACGGCGTTACTTGCCGGAAAAGCCTCTAAGCT
TACAGCAAACGATCTCTACCACTTCTATTCCCATCTCCAACACAACCTTATAACCGTAAT
TTTACTCTTTGCTTTCACCGCTTTCGGTTTGGTTCTCTACATTGTAACCCGGCCCAAACC
GGTTTACCTCGTTGACTACTCGTGCTACCTTCCACCACCGCATCTCAAAGTTAGTGTTTC
CAAGGCGATGGATATTTTCTACCAAATAAGAAAAGCTGATACCTCACGGAACGTGGCATG
CGATGATCCATCCTCGCTTGATTTCCTGAGGAAGATTCAAGAACGTTCAGGTCTAGGTGA
TGAAACGTACAGTCCCCAGGGACTCATTAACGTGCCCCCACAAAAGACCTTTGCAGCTTC
ACGTGAAGAGACAGAGCAGGTAATCATCGGTGCGCTAGAAAAGCTATTCGAGAACACCAA
AGTAAACCCTAGAGAGATTGGTATACTTGTGGTGAACTCAAGCATGTTTAATCCAACTCC
TTCGCTATCTGCGATGGTCGTTAACACTTTCAAGCTCCGAAGCAACATCAAAAGCTTTAG
TCTCGGAGGAATGGGTTGTAGTGCTGGTGTCATCGCCATTGATCTTGCAAAGGACTTGTT
GCATGTTCATAAAAACACTTATGCACTTGTGGTGAGCACTGAGAACATCACTCAAGGCAT
TTATGCTGGCGAAAACAGATCCATGATGGTTAGCAATTGCTTGTTTCGTGTTGGTGGGGC
AGCGATTTTGCTCTCCAACAAACTGGGAGATCGGAGACGGTCCAAGTACAAGCTATGTCA
TACTGTTCGAACGCATACCGGAGCTGATGACAAGTCTTTTCGATGTGTGCAACAAGGAGA
CGATGAGGGCGGTAAAATCGGAGTTTGTCTGTCAAAGGACATAACCGTTGTTGCGGGGAC
AGCGCTTAAGAAAAACATAGCAACGTTGGGTCCGTTGATTCTTCCTTTAAGCGAAAAGTT
TCTGTTTTTAGTTACCTTCATCGCCAAGAAACTTTTGAAGGACAAGATCAAGCACTGTTA
CGTCCCGGATTTCAAGCTTGCTATCGACCATTICTGTATTCATGCGGGAGGCAGAGCCGT
GATCGATGTGCTTGAGAAGAGCTTAGGACTATCGCCAATCGATGTGGAGGCATCTAGATC
AACGTTACATAGATTTGGGAATACTTCGTCTAGCTCAATTTGGTATGAATTGGCATACAT
AGAAGCAAAAGGAAGGATGAAGAAAGGGAATAGAGCTTGGCAGATTGCTTTAGGGTCAGG
GTTTAAGTGTAACAGTGCGGTTTGGGTGGCTCTATGCAATGTCAAGGCTTCGGCGAATAG
TCCTTGGGAACATTGCATCGATAGATATCCGGTTCAAATTGATTCTGATTCATCAAAATC
AGAGACTCATGTCAAAAACGGTCGGACCTAATTCATCTATCTGAGTGTTAACAATTTAGT
GTTTTTTTCTTCTTTCCTTCTTTCTTTTAATTATTGGTTATGATTTGATAATGTTTAATG
TGTTCTTTCTTTTTCGTTATAAATAAAGAAACACATGGGTGTTTCCATTCTTCTATTTAA
GTTGTGGACCGTTTAATAACCTAACTTAGAATTCAATCATCTTTTTATTTTCCTTCTGTT
TTTAAATCTTCCAACCAATCAACGTGTTTTCCATACTAACATAATTATTTAAAACGTGTT
TAAAATTAAACATCTCTTTGGTGTTTCTAGAGGTTTTAGATTGTTTGCTCTGTTTGCCAC
CTGTTTTTTTTCCCCGTCAGCTCTGTTTGCCACCTGTTATTGTTGGAAAAGTTACATTCC
TGTATTTGAGATTTAGATATATAAATGCATATTTGAATTGTTTAGGTATCAAATAAAAAA
AGAAGCAAGGAAGCCCATATAGTTCAATTGATAGTTGAATAATGAAAAATGATATGTGTC
AGAGAGTTGGAGCAGTCCCCGATGAGCCTACATCAGAAAACAAAAAAAAATCATACTGCT
AATATATTTTTTGGCTGAGGCTCACCTTATCTATTAATTATGACATTAGTTACATTGCAC
ATGTAATTTTCAAAAAATGCGGGCTTTAGAGCCTGACCGTCTAGAGAAAGAAGTGGGTGG
TTTAGAAGAGCTTCTTCTACAAATGTAACTAATGTCATTAGTTAATTGTGGCGGCGATCT
ATTAATTATGACATTAGTTACATTGCACATGTAATT,
FAE1-C (Csa12g009060)
>12 dna:chromosome chromosome:Cs:12:
2883224:2890269:−1
SEQ ID NO: 30
AGCATAAAATTAACAAAGAGCAACAGTCAAGCTACAGAATCATAATATATTTTTAGTTTA
TAGGGAAAGCCAACAATCATAAGTGAAAGGTAAGAACGTTATTGGTATAGTACTAATACA
CAGTACTGTAATTCTTAGTATAAATACAGATCCAGTGATTTTTACTAAATTGACTTTAGG
TCCACTGGAGAAACTAAAAAACAATTGGGCCCTAGCCCATTCTTATTTTTACCTCCATAT
CCTCTCAATTAAAATACCTTTTCCTATAAATTTAGAAAAACAGAAACCAATTCAAAAAAA
ATCTACTCTTCTCTATTTCTCCATAAAAACCAAAAAAAACTTATTCACTCTTGCTCATTA
CGTCTCCCATTAGTTGGCCAATGCACATGATGCCAAAATACATTAATGGCGACTTGTTTC
TGTTTTTTAGTTATATCAAACTCCACCCAACCAGGAGAAAAGTTAATGGTGTTTCTGTCC
CTATAAACACAATACATTCCCCTTACGATATTTCTTCCTCTTCATCTCTCTCTCAGTGTC
TATATATACGCGCACACACACCCAGCTTCAGTTTCTTACAAGCATCAGCAACAAGTTTCC
ATGAACTCCCGAAACTACGTCAAGCTTGGTTATCACTACCTGATTACTCACTTCTTGAAG
CTCATGCTCGTCCCTCCTGTAATGGCGGTTTTGTTCACGAACGTCTCCTTATTAACTCTA
AACCAGCTCCAGCTCCAGCTCCAATACAACCTCGTCGGAGTCATCTCCCTCATCACATTC
GCCATATTCGGATGCACAGTCTTCTTCATGTCACGACCTAGATCTGTTTACCTTGTCGAT
TACTCCTGCTACTTACCACCGTCTGATCAAAAAGTTAGCTACCAAAAATTCATGAACAAC
TCAAGTTTGATTCAAGATTTCAGCGAGTCGTCGCTTGAGTTCCAACGAAAGATCTTGGTT
CGATCCGGTCTCGGAGAAGAGACTTATTTACCGGAGTCTATTCACTGCACCCCGCCACGT
CCAACTATGGCCGCTGCGCGTGAGGAAGCAGAGCAGGTAATCTTCGGTGCACTCGATAAC
CTCTTCGAGAATACCAAAATCAATCCTAGAGAGATTGGTGTTCTTGTTGTGAACTGTAGT
TTGTTTAACCCTACGCCTTCTTTATCGGCCATGATTGTTAACAAGTATAAACTTAGAGGA
AACGTCAAGAGCTTTAACCTAGGTGGGATGGGATGTAGTGCTGGTGTTATCGCCGTTGAT
CTCGCTAGTGACATGTTACAGATCCTTAGGAACACTTTTGCTCTTGTGGTTAGTACTGAG
AACATTACTCAGAATTGGTATTTTGGTAACAAGAAAGCAATGTTGATCCCTAATTGCTTG
TTTAGAGTTGGTGGCTCCGCGGTTCTGCTTTCGAACAAGCCTTTGGATCGAAAACGGTCC
AAGTATAAGCTTGTTCATACGGTAAGGACTCATAAAGGATCTGATGAGAAAGCATTCAGT
TGTGTGTACCAAGAACAAGACGAGTGTTTAAAAACCGGAGTTTCTTTGTCGAAAGATCTT
ATGGCTATAGCCGGAGAAGCTTTAAAGACGAACATCACCACTTTGGGTCCTCTGGTTCTT
CCTATAAGCGAGCAGATCCTCTTCTTTGCGACTTTTGTTGCTAAGAGATTGTTCAATGCC
AAGAAGCAGAAGCCTTACATACCAGATTTCAAACTTGCGTTTGATCATTTCTGTATTCAC
GCGGGAGGTAGAGCCGTGATCGATGAACTAGAGAAGAGCTTAAAGCTATCGCCAATACAT
GTCGAGGCGTCTAGAATGACATTGCATAGATTTGGAAACACTTCCTCGAGCTCGATATGG
TATGAATTGGCTTACACGGAAGCTAAAGGAAGAATGAGGAAAGGGAACAGAGTTTGGCAG
ATTGCTTTTGGAAGCGGGTTTAAGTGTAACAGCGCGGTTTGGATGGCTCTCCGCGATGTC
GAGCCCTCGTTTAAAAATCCTTGGGAACATTGCATCGATAGATATCCGGTTAAGATCGAT
CTTTGAACTCGTAAGAACGGTNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNACATGTCGAGGCGTCTAGAATGACA
TTGCATAGATTTGGAAACACTTCCTCGAGCTCGATATGGTATGAATTGGCTTACACGGAA
GCTAAAGGAAGAATGAGGAAAGGGAACAGAGTTTGGCAGATTGCTTTTGGAAGCGGGTTT
AAGTGTAACAGCGCGGTTTGGATGGCTCTCCGCGATGTCGAGCCCTCGTTTAAAAATCCT
TGGGAACATTGCATCGATAGATATCCGGTTAAGATCGATCTTTGAACTCGTAAGAACGGT
GGATTGGTCCGGAAACATGGTTAGTCCTCCATGTACCAAAAAAGAAAGTGAACTTATATC
TCTAGTTCTTTACTAAGGGGTCAAGAAATCAGTTGAAGGTTAATGTATGTTTATATATGT
TGTGTATAAAGGTAACCCTTAAATGACCAAGACTGTTGACCTATTAAAATTTAAAATTTA
AGTTCTCTCGAAAAATTAAATCGTTTGTAAAATAAAAATTGTGAGTTAGAAAAATCAAAA
CAATTTACTTACCTACAAGAACCTTAAAATTAAACTTAAGTATTAAATTTTGAATACATG
AAAAAATGTTAAACTTATGACACCAAAATTTACGAACTCTGAGGACGGAAATGCTTAATT
CAGTTTGTTTGTTTTTTTGTTTTGTAAATCAATCTGGTTTGTTTTCAATGTATCTAAACT
AAAGTCGGGTTATGACACCAACACCAAGCTTATGGAGACAGGATGTTAAGTCGGGTTAGT
CATCAAGGTATTCAAATAAGTAATAACTTTTAAGTTTTAGAACTTTTTTGTCAGTAAAAT
ATTTTGTATAATAAAAAAGTTTAGTTAAAACATTAAAACAGTTTTTACTGAAAATATTAT
TTGTGAAAAAACTTCTTTTAAGTGTTAAATTTAGAACACGTAAAAACATGTTAGACTTAT
GACACCAAGCTTATAGACCCGAAAAGGAAACTGTGATTGTTTCCTTTTCCCCACGAGTTC
TAAGTTGTTTCCTTTACTTATTTGGGTTGCAACCAATATAAAAAGAAAGAAAAATGGAAA
TTTATCACAAAAAGGAGAGTCGAGATCTCTAGAGTTACATTCATATGTGCTAGTGTCTGA
CCTTTTGCCTTAATTTATAGGCACTAATAAAAAAACTACCAAAAAAAAAATTGAAGAAGA
ACAAAAGGTTAAACAATCACTGGATCAGTGTGCTCATGGTGTTCCAGGTCCAAAACATGT
GTCACGAGATTAAAAAAGAGAGAAACACAACTGAAACTATTCACCAAGATCACAACTATA
TATATAAAGGACTTATAAAAAATCAATTTGAAAGTGGTTAAACTAAACGATTATAAGTGT
GATTGCACTTACCCCCTTATAGGTTTGGTGAATCTTATTAGAGATAACTTATTTTTAAGA
TAGTTGCAATTAAAAAAAAAAAAAAATTGTCCGGATAGTTTGATGCAATTAATGAGTGTT
CTATATGGTCTGATTCTTAATATTTATGCAAATATTATAGTATTTCAAAGTATTTGGCCT
TAACTGACAGACTTTGCAAATAAAACTCATTCCCGAGAAACATTCATCCCATAATTGGTA
TTTAGTCAGAGGCTAATCGACTATGGCCTTTCAGCCAATCAAAGCTACGAACACGAATCC
CCCTAAAACATCCTCAAGTATTTATTTAATACATCGTATTGAGCACCACTCATAAACTAA
TTCCATACATTTATCATACTGTTTATTTGTAATAATAAAAGCAGCAACATATTGTAGTTT
GTAGGCAATAAGAAACAAAACAAAACATTTTTTTTTCTCTCCAAATTTTCAAAATTGGAA
AACGAAACTTGGACCTTCAATACTTATATATTATATTTGCAATATAAAATTGCAGAGTGG
ACTATTTCCCTTATTTTGGCAACTTTCAGTGGACTAGTAATTTATTTCAATGTGTATGCT
TGCATGAGTGTGAATATACACATGTCTATATGCATGCCTGCAAATCGTAACGGACCACAA
AAAAGGATCCATACAAATATACCTCTCAACGGCTCCTCTCTATTATGCTCTCCGACACAA
ACTGAGAAATGACGTCCGTTAACGCAAAGCTCCTTTACCATTACGTCCTAACCAACTTTT
TCAACCTTTGCTTGTTTCCGTTAACGGCGTTACTTGCCGGAAAAGCCTCTACGCTTACCA
CAAACGATCTCTACCACTTCTATTCCCATCTCCAACACAACCTTGTAACCGTAATTTTAC
TCTTTGCTTTCTCCTCTTTCGGTTTGGTTCTCTACGTTGTAACCCGGCGCAGACCGGTTT
ACCTCGTTGACTACTCGTGCTACCTTCCACCACCGCATCTCAAAGTTAGTGTTTCTAAGG
TCATGGATATTTTCTACCAAATAAGAAAAGCTGATACCTCACGAAACGTGGCATGCGATG
ATCCATCCTCGCTTGATTTCCTGAGGAAGATTCAAGAACGTTCAGGTCTAGGTGATGAAA
CCTACAGTCCCCCGGGACTCATTCACGTGCCCCCACAAAAGACTTTTGCAGCTTCACGTG
AAGAGACAGAGCAGGTAATCATCGGTGCGTTAGAAAAGTTATTCGAGAACACCAAAGTTA
ACCCTAGAGAGATTGGTATACTTGTGGTCAACTCAAGCATGTTTAATCCAACTCCTTCGC
TATCTGCGATGGTCGTTAACACTTTCAAGCTCCGAAGCAACATCAAAAGCTTTAGTCTCG
GAGGAATGGGTTGTAGTGCTGGTGTCATCGCCATTGATCTTGCAAAGGACTTGTTGCATG
TTCATAAAAACACTTATGCACTTGTGGTGAGCACTGAGAACATCACTCAAGGCATTTATG
CTGGCGAAAATAGATCCATGATGGTTAGCAATTGCTTGTTTCGTGTTGGTGGGGCAGCGA
TTTTGCTATCCAACAAGCCGGGAGATCGGAGACGGTCCAAGTACAAGCTATGTCACACGG
TTCGGACTCATACCGGAGCTGATGACAAGTCTTTTCGATGTGTGCAACAAGGAGACGATG
AGAGCGGTAAAATCGGAGTTTGTCTGTCAAAGGACATAACAGTTGTTGCGGGGACAGCGC
TTAAGAAAAACATAGCAACGTTAGGTCCGTTGATTCTTCCTTTAAGCGAAAAGTTTCTGT
TCTTAGTTACCTTCATCGCCAAGAAACTTTTGAAGGACAAGATCAAGCACTATTACGTCC
CGGATTTCAAGCTTGCTATTGACCATTTCTGTATTCATGCCGGAGGCAGAGCCGTAATCG
ATGTGCTTGAGAAGAGCTTAGGACTATCGCCAATCGATGTGGAGGCATCTAGATCAACGT
TACATAGATTTGGGAATACTTCGTCTAGCTCAATTTGGTATGAATTGGCATACATAGAAG
CAAAAGGAAGGATGAAGAAAGGGAATAGAGCTTGGCAGATTGCTTTAGGGTCAGGGTTTA
AGTGTAACAGTGCGGTTTGGGTGGCTCTATGCAATGTCAAGGCTTCCGCGAATAGTCCTT
GGGAACATTGCATCGATAGATATCCGGTTCAAATTGATTCTGATTCATCAAAATCAGAGA
CTCATGTCAAAAACGGTCGGACCTAATTCATCTATCTGAGTGTTAACCATTTACTGTTTT
TTATTTTTCTTTCCTTCTTTCTTTTATTGGTTATGATTTGATAATGTTTAATGATGTTCT
TTCTCTCTTTTTCGTTATAAACTTATATAAATAAAGAAACACATGGGTGTTTTCATTCTT
CTATTTAAGTAGTGGACCATTTTATAACCTAACTTATAACTCAATCATCATTTTTTTTTT
TTTCTGTTTTCAACTCTTCCAACCAATCAACGTTTTTTTCATACTAACATAATTATTAAA
ATGTGTTTAAAATTAAACATCTCTTTGGTGTTTCTAGAGGTTTTAGATTGTTTGCTCTGT
TTGCCAAATGTTTTATTTTTTTCCCCTTCAGCTCTGTTTGCCACCTGTTATTGTTGGAAA
AGTTACATTCCTGTTTTTGAGATTTAGATATATAAATGCATATTTGAATTGTTTAGGTAT
CAATATTAAAAAAAAAAAAAAGCAAGGAAGCCCATATAGTTCAATTGATAGTTGAATAAA
TGAAAAATGATATGTGTCACAGTGTCAGAGAGTTGGAGCAGTCCCCCATGAGCCTACATC
AGAAAACAAAAAAAAAATCATACTAC
FAE1-A(Csa11g007400) - amino acid
SEQ ID NO: 31
MTSVNAKLLYHYVLTNFFNLCLFPLTALLAGKASRLTSNDLYHFYSHLOHNLITVILLFA
FTAFGLVLYIVTRPKPVYLVDYSCYLPPPHLKVSVSKAMDIFYQIRKADTSRNVACDDPS
SLDFLRKIQERSGLGDETYSPQGLINVPPRKTFAASREETEQVIIGALDKLFENTKVNPR
EIGILVVNSSMFNPTPSLSAMVVNTFKLRSNIKSFSLGGMGCSAGVIAIDLAKDLLHVHK
NTYALVVSTENITQGIYAGENRSMMVSNCLFRVGGAAILLSNKPGDRRRSKYKLCHTVRT
HTGADDMSFRCVQQGDDESGKIGVCLSKDITVVAGIALKKNIATLGPLILPLSEKFLFLV
TFIAKKLLKDKIKHYYVPDFKLAIDHFCIHAGGRAVIDVLEKSLGLSPIDVEASRSTLHR
FGNTSSSSIWYELAYIEAKGRMKKGNRAWQIALGSGFKCNSAVWVALCNVKASANSPWEH
CIDRYPVOIDSGSSKSDTHVKNASRLTSNDLYHFYSHLOHNLITVILLFAFTAFGLVLYI
VTRPKPVYLVDYSCYLPPPHLKVSVSKAMDIFYQIRKADTSRNVACDDPSSLDELRKIQE
RSGLGDETYSPQGLINVPPRKTFAASREETEQVIIGALDKLFENTKVNPREIGILVVNSS
MFNPTPSLSAMVVNTFKLRSNIKSFSLGGMGCSAGVIAIDLAKDLLHVHKNTYALVVSTE
NITQGIYAGENRSMMVSNCLFRVGGAAILLSNKPGDRRRSKYKLCHTVRTHTGADDMSFR
CVQQGDDESGKIGVCLSKDITVVAGIALKKNIATLGPLILPLSEKFLFLVTFIAKKLLKD
KIKHYYVPDFKLAIDHFCIHAGGRAVIDVLEKSLGLSPIDVEASRSTLHREGNTSSSSIW
YELAYIEAKGRMKKGNRAWQIALGSGFKCNSAVWVALCNVKASANSPWEHCIDRYPVQID
SGSSKSDTHVKNGRT
FAE1-B(Csa10g007610) - amino acid
SEQ ID NO: 32
MTSVNAKLLYHYVLTNFFNLCLFPLTALLAGKASKLTANDLYHFYSHLQHNLITVILLFA
FTAFGLVLYIVTRPKPVYLVDYSCYLPPPHLKVSVSKAMDIFYQIRKADTSRNVACDDPS
SLDFLRKIQERSGLGDETYSPQGLINVPPQKTFAASREETEQVIIGALEKLFENTKVNPR
EIGILVVNSSMFNPTPSLSAMVVNTFKLRSNIKSFSLGGMGCSAGVIAIDLAKDLLHVHK
NTYALVVSTENITQGIYAGENRSMMVSNCLFRVGGAAILLSNKLGDRRRSKYKLCHTVRT
HTGADDKSFRCVQQGDDEGGKIGVCLSKDITVVAGTALKKNIATLGPLILPLSEKFLFLV
TFIAKKLLKDKIKHCYVPDFKLAIDHFCIHAGGRAVIDVLEKSLGLSPIDVEASRSTLHR
FGNTSSSSIWYELAYIEAKGRMKKGNRAWQIALGSGFKCNSAVWVALCNVKASANSPWEH
CIDRYPVQIDSDSSKSETHVKNGRT
FAE1-C(Csa12g009060) - amino acid
SEQ ID NO: 33
MNSRNYVKLGYHYLITHFLKLMLVPPVMAVLFTNVSLLTLNQLQLQLQYNLVGVISLITE
AIFGCTVFFMSRPRSVYLVDYSCYLPPSDQKVSYQKFMNNSSLIQDFSESSLEFORKILV
RSGLGEETYLPESIHCTPPRPTMAAAREEAEQVIFGALDNLFENTKINPREIGVLVVNCS
LFNPTPSLSAMIVNKYKLRGNVKSENLGGMGCSAGVIAVDLASDMLQILRNTFALVVSTE
NITQNWYFGNKKAMLIPNCLFRVGGSAVLLSNKPLDRKRSKYKLVHTVRTHKGSDEKAFS
CVYQEQDECLKTGVSLSKDLMAIAGEALKTNITTLGPLVLPISEQILFFATFVAKRLENA
KKQKPYIPDFKLAFDHFCIHAGGRAVIDELEKSLKLSPIHVEASRMTLHREGNTSSSSIW
YELAYTEAKGRMRKGNRVWQIAFGSGFKCNSAVWMALRDVEPSFKNPWEHCIDRYPASRM
TLHRFGNTSSSSIWYELAYTEAKGRMRKGNRVWQIAFGSGFKCNSAVWMALRDVEPSFKN
PWEHCIDRYPLLYHYVLTNFFNLCLFPLTALLAGKASTLTINDLYHFYSHLOHNLVTVIL
LFAFSSFGLVLYVVTRRRPVYLVDYSCYLPPPHLKVSVSKVMDIFYQIRKADTSRNVACD
DPSSLDFLRKIQERSGLGDETYSPPGLIHVPPQKTFAASREETEQVIIGALEKLFENTKV
NPREIGILVVNSSMENPTPSLSAMVVNTFKLRSNIKSFSLGGMGCSAGVIAIDLAKDLLH
VHKNTYALVVSTENITQGIYAGENRSMMVSNCLFRVGGAAILLSNKPGDRRRSKYKLCHT
VRTHTGADDKSFRCVQQGDDESGKIGVCLSKDITVVAGTALKKNIATLGPLILPLSEKFL
FLVTFIAKKLLKDKIKHYYVPDFKLAIDHFCIHAGGRAVIDVLEKSLGLSPIDVEASRST
LHRFGNTSSSSIWYELAYIEAKGRMKKGNRAWQIALGSGFKCNSAVWVALCNVKASANSP
WEHCIDRYPVQIDSDSSKSETHVKNGRT
HBFD1 (Adonis aestivalis) 407aa
SEQ ID NO: 34:
MAPVLLGLKPTLSTGSVVKETNVGSTLASPLNKTQNSRVLVLGGTGKVGGSTALALSKFS
PDLRLVIGGRNREKGDAVVSKLGENSEFVEVNVDSVRSLESALEDVDLVVHAAGPFQQAE
KCTVLEAAISTRTAYVDVCDNTSYSMQAKSFHDKAVAANVPAITTAGIFPGVSNVIAAEL
VRSARDENTEPQRLRFSYFTAGSGGAGPTSLVTSFLLLGEEVVAYSEGEKVELKPYTGKL
NIDFGKGVGKRDVYLWNLPEVRSGHEILGVPTVSARFGTAPFFWNWAMVAMTTLLPPGIL
RDRNKIGMLANFVYPSVQIFDGIAGECLAMRVDLECANGRNTFGILSHERLSVLVGTSTA
VFAMAILEGSTQPGVWFPEEPGGIAISDRELLLQRASQGAINFIMKQ
Keto-2 (Adonis aestivalis) 309aa
SEQ ID NO: 35
MAAAISVFSSGYSFYKNLLLDSKPNILKPPCLLFSPVVIMSPMRKKKKHGDPCICSVAGR
TRNLDIPQIEEEEENVEELIEQTDSDIVHIKKTLGGKQSKRPTGSIVAPVSCLGILSMIG
PAVYFKFSRLMEGGDIPVAEMGITFATFVAAAVGTEFLSAWVHKELWHESLWYIHKSHHR
SRKGRFEFNDVFAIINALPAIALINYGFSNEGLLPGACFGVGLGTTVCGMAYIFLHNGLS
HRRFPVWLIANVPYFHKLAAAHQIHHSGKFQGVPFGLFLGPKELEEVRGGTEELERVISR
TTKRTQPST
ZmPhys (Zea mays) 410aa
SEQ ID NO: 36
MAIILVRAASPGLSAADSISHQGTLQCSTLLKTKRPAARRWMPCSLLGLHPWEAGRPSPA
VYSSLAVNPAGEAVVSSEQKVYDVVLKQAALLKRQLRTPVLDARPQDMDMPRNGLKEAYD
RCGEICEEYAKTFYLGTMLMTEERRRAIWAIYVWCRRTDELVDGPNANYITPTALDRWEK
RLEDLFTGRPYDMLDAALSDTISRFPIDIQPFRDMIEGMRSDLRKTRYNNFDELYMYCYY
VAGTVGLMSVPVMGIATESKATTESVYSAALALGIANQLTNILRDVGEDARRGRIYLPQD
ELAQAGLSDEDIFKGVVTNRWRNFMKRQIKRARMFFEEAERGVTELSQASRWPVWASLLL
YRQILDEIEANDYNNFTKRAYVGKGKKLLALPVAYGKSLLLPCSLRNGQT
HBFD1 CDS sequence
SEQ ID NO: 37
ATGGCTCCAGTGCTTTTGGGTTTGAAGCCTACTTTGTCAACTGGTTCTGTGGTGAAAGAA
ACAAACGTTGGATCAACACTTGCTTCACCTTTGAACAAAACTCAGAACTCAAGGGTGCTT
GTGTTGGGTGGTACAGGAAAAGTGGGTGGATCTACAGCACTTGCTCTTTCAAAGTTCTCT
CCAGACCTTAGGTTGGTGATTGGAGGAAGGAACAGGGAGAAGGGTGACGCAGTGGTGTCA
AAGTTGGGAGAAAATTCTGAGTTCGTTGAGGTTAATGTGGATTCTGTGAGGTCACTTGAG
TCTGCACTTGAGGACGTTGACCTTGTGGTTCACGCTGCAGGTCCTTTCCAGCAAGCAGAG
AAGTGCACAGTGTTGGAGGCTGCTATCTCTACTAGGACTGCTTACGTGGACGTGTGCGAC
AACACTTCTTACTCTATGCAGGCTAAGTCATTCCACGACAAGGCAGTGGCTGCAAACGTG
CCTGCTATCACAACTGCAGGAATCTTCCCTGGAGTGTCTAACGTGATCGCAGCAGAGTTG
GTGAGGTCTGCAAGGGATGAGAACACAGAGCCTCAAAGGTTGAGGTTCTCTTACTTCACA
GCTGGATCTGGTGGAGCTGGTCCAACATCATTGGTGACTTCATTCTTGTTGTTGGGTGAG
GAGGTGGTGGCTTACTCTGAGGGAGAGAAGGTTGAGTTGAAGCCATATACAGGAAAGTTG
AATATTGACTTTGGAAAAGGTGTGGGAAAGAGAGACGTGTACTTGTGGAACTTGCCTGAG
GTGAGGTCTGGACACGAGATCCTTGGAGTGCCTACTGTGTCTGCTAGGTTCGGTACAGCT
CCATTCTTCTGGAACTGGGCTATGGTGGCAATGACTACATTGCTTCCTCCTGGTATCTTG
AGAGACAGGAATAAGATCGGAATGTTGGCTAATTTTGTTTACCCTTCTGTGCAGATCTTC
GACGGTATCGCAGGAGAGTGCCTTGCTATGAGGGTTGACTTGGAGTGCGCAAACGGAAGA
AATACTTTCGGTATCTTGTCACACGAAAGGTTGTCTGTGCTTGTGGGTACATCTACAGCT
GTGTTCGCTATGGCAATCCTTGAGGGTTCTACTCAGCCTGGTGTTTGGTTCCCTGAGGAG
CCTGGAGGTATCGCAATCTCTGACAGGGAGCTTCTTTTGCAGAGGGCATCACAGGGAGCA
ATCAACTTCATTATGAAGCAGTGA
Keto 2 CDS sequence
SEQ ID NO: 38
ATGGCAGCTGCTATTTCAGTGTTCTCTTCAGGATACTCATTCTACAAGAATTTGTTGTTG
GACTCAAAGCCAAACATCCTTAAGCCTCCTTGCCTTCTTTTCTCTCCTGTGGTGATCATG
TCTCCTATGAGGAAGAAGAAGAAGCACGGAGATCCATGTATCTGCTCTGTGGCAGGTAGG
ACAAGGAACTTGGACATCCCACAGATCGAAGAGGAGGAAGAGAACGTGGAGGAGTTGATC
GAGCAGACTGACTCTGACATCGTTCACATCAAGAAGACTTTGGGAGGTAAGCAGTCAAAG
AGGCCAACTGGATCTATCGTGGCTCCTGTGTCATGCTTGGGTATCTTGTCAATGATCGGA
CCAGCTGTGTATTTCAAGTTCTCTAGGCTTATGGAGGGAGGAGACATCCCTGTGGCTGAG
ATGGGAATCACATTCGCAACTTTCGTTGCTGCTGCTGTGGGAACAGAGTTCTTGTCAGCA
TGGGTGCACAAGGAGTTGTGGCACGAGTCTTTGTGGTACATCCACAAGTCACATCACAGG
TCTAGGAAGGGTAGGTTTGAGTTCAACGACGTGTTCGCTATCATTAACGCCCTTCCAGCT
ATCGCATTGATCAATTACGGTTTCTCAAACGAGGGATTGTTGCCTGGTGCTTGCTTCGGA
GTGGGATTGGGAACTACAGTGTGCGGTATGGCTTACATCTTCCTTCACAACGGATTGTCT
CACAGGAGATTCCCAGTGTGGCTTATTGCAAACGTGCCATACTTCCACAAGTTGGCAGCT
GCTCACCAGATCCATCACTCAGGAAAGTTTCAAGGAGTGCCTTTCGGATTGTTCTTGGGT
CCAAAGGAGCTTGAGGAGGTGAGAGGAGGAACTGAGGAGTTGGAGAGAGTGATTTCTAGG
ACTACAAAGAGGACTCAACCATCTACTTGA
ZmPhys CDS sequence
SEQ ID NO: 39
ATGGCTATTATTTTGGTTAGAGCTGCTTCACCTGGACTTTCTGCTGCTGATTCTATTTCT
CATCAAGGTACTTTGCAATGTTCAACACTTTTGAAAACTAAAAGACCAGCAGCTAGAAGA
TGGATGCCTTGTTCTTTGTTGGGACTTCATCCTTGGGAAGCTGGTAGACCTTCTCCAGCT
GTTTATTCTTCTCTTGCTGTTAACCCAGCTGGAGAAGCTGTTGTTTCTTCAGAACAGAAG
GTTTACGATGTTGTTCTTAAGCAGGCTGCTTTGTTGAAGAGACAACTTAGAACTCCAGTT
TTGGATGCAAGACCTCAAGATATGGATATGCCTAGAAATGGACTTAAGGAGGCATATGAT
AGATGTGGAGAAATCTGTGAAGAATACGCTAAAACATTTTATTTGGGTACTATGCTTATG
ACTGAGGAGAGAAGAAGGGCTATTTGGGCTATCTACGTTTGGTGTAGAAGAACTGATGAG
CTTGTTGATGGACCAAACGCTAACTACATCACTCCTACTGCTCTTGATAGATGGGAGAAA
AGATTGGAGGATCTTTTTACTGGAAGACCTTACGATATGTTGGATGCTGCTCTTTCAGAT
ACTATCTCAAGATTCCCTATCGATATCCAACCTTTCAGAGATATGATCGAGGGTATGAGA
TCTGATCTTAGAAAGACTAGATACAACAACTTCGATGAATTGTACATGTACTGTTACTAT
GTTGCTGGTACAGTTGGTTTGATGTCTGTTCCTGTTATGGGTATCGCTACTGAGTCTAAA
GCTACTACAGAATCTGTTTATTCTGCTGCTCTTGCTTTGGGTATTGCTAATCAACTTACA
AACATCCTTAGAGATGTTGGAGAAGATGCTAGAAGGGGAAGAATCTACCTTCCACAGGAT
GAACTTGCTCAAGCTGGGTTGTCAGATGAGGATATCTTCAAGGGTGTTGTTACTAACAGA
TGGAGAAACTTCATGAAGAGACAGATCAAGAGAGCTAGAATGTTCTTCGAGGAAGCTGAA
AGAGGTGTTACTGAATTGTCTCAAGCATCAAGATGGCCTGTTTGGGCTTCTCTTCTTTTG
TACAGACAAATCCTTGATGAGATCGAGGCTAACGATTATAACAACTTCACAAAGAGAGCT
TATGTTGGAAAGGGAAAGAAACTCTTGGCCCTTCCTGTTGCTTATGGAAAAAGCCTTTTG
CTTCCTTGTTCATTGAGAAATGGTCAGACATAA

1. A recombinant plant, part thereof or plant cell, wherein the plant, part thereof or plant cell comprises at least one nucleic acid sequence encoding a Δ6-elongase, a Δ5-desaturase and a Δ6-desaturase, and wherein the plant, part thereof or plant cell has reduced expression or activity of a gene encoding an enzyme involved in the synthesis of very long chain fatty acids (VLCFAs).

2. The recombinant plant, part thereof or plant cell of claim 1, wherein the recombinant plant, part thereof or plant cell further comprises at least one nucleic acid sequence encoding a hydroxy-beta-ring 4-dehydrogenase (HBFD1) and/or at least one nucleic acid sequence encoding Keto2 and/or at least one nucleic acid sequence encoding a phytoene synthase, wherein optionally the HBFD1 nucleic acid sequence encodes an amino acid sequence as defined in SEQ ID NO: 34 or a functional variant thereof, and wherein optionally the Keto2 nucleic acid sequence encodes an amino acid sequence as defined in SEQ ID NO: 35 or a functional variant thereof, and wherein optionally the phytoene synthase nucleic acid sequence encodes an amino acid sequence as defined in SEQ ID NO: 36 or a functional variant thereof.

3.-7. (canceled)

8. The recombinant plant, part thereof or plant cell of claim 1, wherein the Δ6-elongase is derived from Physcomitrella patens; the Δ5-desaturase is derived from Thraustochytrium, and the Δ6-desaturase is derived from Ostreococcus tauri or Mantoniella squamata and wherein optionally the Δ6-elongase nucleic acid sequence encodes an amino acid sequence as defined in SEQ ID NO: 4 or a functional variant thereof, and/or wherein optionally the Δ5-desaturase nucleic acid sequence encodes an amino acid sequence as defined in SEQ ID NO: 6 or a functional variant thereof and/or wherein optionally the Δ6-desaturase nucleic acid sequence encodes an amino acid sequence as defined in SEQ ID NO: 2, 22 or 24 or a functional variant thereof.

9.-14. (canceled)

15. The recombinant plant, part thereof or plant cell of claim 1, further comprising a nucleic acid sequence encoding a Δ15-desaturase and/or a ω3-desaturase and/or a Δ12-desaturase enzyme, and/or a Δ5-elongase and/or a Δ4desaturase; wherein optionally the Δ15-desaturase is derived from Perilla frutescens, optionally the ω3-desaturase is derived from Hyaloperonospora parasitica or Phytophora infestans; optionally the Δ12-desaturase is derived from Phytophora sojae, optionally the Δ5-elongase is derived from Ostreococcus tauri and optionally the Δ4desaturase is derived from Ostreococcus RCC809; and wherein
a. optionally the nucleic acid sequence encoding a ω3-desaturase encodes an amino acid sequence as defined in SEQ ID NO: 12 or 14 or a functional variant thereof, and
b. optionally the nucleic acid sequence encoding a Δ15-desaturase encodes an amino acid sequence as defined in SEQ ID NO: 20 or a functional variant thereof,
c. optionally the nucleic acid sequence encoding a Δ4-desaturase encodes an amino acid sequence as defined in SEQ ID NO: 16 or 18 or a functional variant thereof, and
d. optionally the nucleic acid sequence encoding a Δ12-desaturase encodes an amino acid sequence as defined in SEQ ID NO: 10 or a functional variant thereof, and
e. optionally the nucleic acid sequence encoding a Δ5-elongase encodes an amino acid sequence as defined in SEQ ID NO: 8 or a functional variant thereof.

16.-20. (canceled)

21. The recombinant plant, part thereof or plant cell of claim 1, wherein the plant, part thereof or cell comprises at least one mutation in a gene encoding an enzyme involved in the synthesis of VLCFAs, wherein optionally the plant, part thereof or plant cell comprises at least one mutation in a gene encoding fatty acid elongase 1 (FAE1), wherein optionally the mutation is a loss-of-function mutation.

22.-28. (canceled)

29. The recombinant plant, part thereof or plant cell of claim 1, wherein the nucleic acid sequences are each operably linked to a regulatory sequence, where the regulatory sequence is selected from the group consisting of: the unknown seed protein seed-specific promoter, the Napin seed specific promoter, the 25 seed storage protein (Conlinin) promoter, the 11S seed storage protein (Glycinin) promoter, the sucrose-binding protein promoter and the Arcelin-5 seed storage protein promoter.

30. The recombinant plant, part thereof or plant cell of claim 1, further comprising a nucleic acid sequence encoding resistance to at least one herbicide.

31. (canceled)

32. The recombinant plant, part thereof or plant cell of claim 1, wherein the part thereof is a seed oil, and wherein the seed oil is characterised by increased EPA and/or DHA, wherein EPA constitutes at least 5% (mole %) of the total fatty acid content of said oil, and wherein DHA constitutes at least 5% (mole %) of the total fatty acid content of said oil and
wherein the seed oil has a gondoic acid (GA) content of 10% (mole %) or less based on the total fatty acid content of the seed oil; and/or
wherein the seed oil has a ketocarotenoid content of at least 100 mg per kg of said seed oil; wherein optionally
the combined total of EPA and DHA is more than 13% (mole %) of the total fatty acid content of the seed oil; and
wherein optionally the amount of total omega-3 fatty acids is increased and/or the amount of total omega-6 fatty acids is decreased in the seed oil, wherein preferably the amount of total omega-3 fatty acids is at least 30% (mole %) of the total fatty acid content present in said oil and/or the amount of total omega-6 fatty acids is less than 30% (mole %) of the total fatty acid content present in said oil.

33.-41. (canceled)

42. The recombinant plant, part thereof or plant cell of claim 1 wherein the plant is selected from the family Brassicaceae, optionally wherein the plant, part thereof or plant cell is Camelina.

43.-46. (canceled)

47. A method of producing a recombinant plant, part thereof or plant cell with increased omega-3 LC-PUFAs content, the method comprising cultivating the recombinant plant, part thereof or cell of claim 1 under conditions which allow the production of one or more omega-3 LC-PUFAs, and obtaining said omega-3 LC-PUFAs from the plant, part thereof or cell.

48.-49. (canceled)

50. The method of claim 47, wherein the method increases the amount of triacylglycerol (TAG) species of 56 carbons and above, wherein optionally the method comprises increasing the amount of 62:16, 64:14, 64:17 and 66:17 TAG species.

51. (canceled)

52. The method of claim 47, wherein the method comprises increasing the amount of one or more of EPA, DHA and DPA in TAG species with 58 or more carbons.

53.-54. (canceled)

55. A method for producing plant seed oil, comprising growing a plant, part thereof or cell according to claim 1 under conditions wherein said nucleic acid sequences are expressed and a plant seed oil is produced in said plant, part thereof or cell.

56. (canceled)

57. A plant seed oil, comprising:
EPA and/or DHA, wherein EPA constitutes at least 5% (mole %) of the total fatty acid content present in said oil; and wherein DHA constitutes at least 5% (mole %) of the total fatty acid content present in said oil; and
wherein the oil has a gondoic acid (GA) content of 10% (mole %) or less based on the total fatty acid content present in said oil, and/or wherein the oil has a ketocarotenoid content of at least 100 mg per kg of said oil.

58. A plant seed oil according to claim 57, wherein DHA constitutes at least 5% (mole %), preferably 6% (mole %) or more, more preferably 7% (mole %) or more, even more preferably 10% (mole %) or more, yet even more preferably 12% (mole %), and most preferably 15% (mole %) or more of the total fatty acid content present in said oil.

59. A plant seed oil according to claim 57, wherein EPA constitutes at least 5% (mole %), more preferably 7% (mole %) or more, even more preferably 8% (mole %) or more, yet even more preferably 9% (mole %) or more, and most preferably 10% (mole %) or more of the total fatty acid content present in said oil.

60. A plant seed oil according to claim 57, wherein GA constitutes 8% (mole %) or less, preferably 7% (mole %) or less, more preferably 6% (mole %) or less, even more preferably 5% (mole %) or less, yet even more preferably 4% (mole %) or less of the total fatty acid content present in said oil.

61. A plant seed oil according to claim 57, wherein the combined total of EPA and DHA constitutes more than 13% (mole %), preferably more than 15% (mole %), more preferably more than 17% (mole %), even more preferably more than 20% (mole %), and yet even more preferably more than 25% (mole %) of the total fatty acid content present in said oil.

62. A plant seed oil according to claim 57, wherein the ketocarotenoid content is at least 100 mg per kg of said oil, preferably at least 150 mg per kg of said oil, more preferably at least 200 mg per kg of the oil, even more preferably at least 300 mg per kg of the oil, yet even more preferably at least 400 mg per kg of the oil, and most preferably at least 500 mg per kg of the oil, and optionally wherein the plant seed oil comprises astaxanthin.

63.-67. (canceled)


SEQUENCE LISTING (39 sequences)

SEQ ID NO: 1 - DNA, 1371 bp, Ostreococcus tauri
atgtgtgttgagaccgagaacaacgatggaatccctactgtggagatcgctttcgatggagagagagaaagagctgaggctaacgtgaagttgtctgctgagaagatggaacctgctgctttggctaagaccttcgctagaagatacgtggttatcgagggagttgagtacgatgtgaccgatttcaaacatcctggaggaaccgtgattttctacgctctctctaacactggagctgatgctactgaggctttcaaggagttccaccacagatctagaaaggctaggaaggctttggctgctttgccttctagacctgctaagaccgctaaagtggatgatgctgagatgctccaggatttcgctaagtggagaaaggagttggagagggacggattcttcaagccttctcctgctcatgttgcttacagattcgctgagttggctgctatgtacgctttgggaacctacttgatgtacgctagatacgttgtgtcctctgtgttggtttacgcttgcttcttcggagctagatgtggatgggttcaacatgagggaggacattcttctttgaccggaaacatctggtgggataagagaatccaagctttcactgctggattcggattggctggatctggagatatgtggaactccatgcacaacaagcaccatgctactcctcaaaaagtgaggcacgatatggatttggataccactcctgctgttgctttcttcaacaccgctgtggaggataatagacctaggggattctctaagtactggctcagattgcaagcttggaccttcattcctgtgacttctggattggtgttgctcttctggatgttcttcctccatccttctaaggctttgaagggaggaaagtacgaggagcttgtgtggatgttggctgctcatgtgattagaacctggaccattaaggctgttactggattcaccgctatgcaatcctacggactcttcttggctacttcttgggtttccggatgctacttgttcgctcacttctctacttctcacacccatttggatgttgttcctgctgatgagcatttgtcttgggttaggtacgctgtggatcacaccattgatatcgatccttctcagggatgggttaactggttgatgggatacttgaactgccaagtgattcatcacctcttcccttctatgcctcaattcagacaacctgaggtgtccagaagattcgttgctttcgctaagaagtggaacctcaactacaaggtgatgacttatgctggagcttggaaggctactttgggaaacctcgataatgtgggaaagcactactacgtgcacggacaacattctggaaagaccgcttga

SEQ ID NO: 2 - protein, 456 aa, Ostreococcus tauri
MCVETENNDGIPTVEIAFDGERERAEANVKLSAEKMEPAALAKTFARRYVVIEGVEYDVTDFKHPGGTVIFYALSNTGADATEAFKEFHHRSRKARKALAALPSRPAKTAKVDDAEMLQDFAKWRKELERDGFFKPSPAHVAYRFAELAAMYALGTYLMYARYVVSSVLVYACFFGARCGWVQHEGGHSSLTGNIWWDKRIQAFTAGFGLAGSGDMWNSMHNKHHATPQKVRHDMDLDTTPAVAFFNTAVEDNRPRGFSKYWLRLQAWTFIPVTSGLVLLFWMFFLHPSKALKGGKYEELVWMLAAHVIRTWTIKAVTGFTAMQSYGLFLATSWVSGCYLFAHFSTSHTHLDVVPADEHLSWVRYAVDHTIDIDPSQGWVNWLMGYLNCQVIHHLFPSMPQFRQPEVSRRFVAFAKKWNLNYKVMTYAGAWKATLGNLDNVGKHYYVHGQHSGKTA

SEQ ID NO: 3 - DNA, 873 bp, Physcomitrella patens
atggaagttgttgagaggttctacggagagttggatggaaaggtttcccaaggagtgaacgctttgttgggatctttcggagttgagttgactgataccccaactactaagggattgccactcgttgattctccaactccaattgtgttgggagtgtctgtttacttgaccatcgtgatcggaggattgctttggatcaaggctagagatctcaagccaagagcttctgagccattcttgttgcaagctttggtgttggtgcacaacttgttctgcttcgctttgtctctttacatgtgcgtgggtatcgcttaccaagctatcacctggagatattccttgtggggaaacgcttataacccaaagcacaaggagatggctatcctcgtttacctcttctacatgtccaagtacgtggagttcatggataccgtgatcatgatcctcaagagatccaccagacagatttctttcctccacgtgtaccaccattcttctatctcccttatctggtgggctattgctcatcatgctccaggaggagaggcttattggagtgctgctctcaactctggagtgcatgtgttgatgtacgcttactacttcttggctgcttgcttgagatcttccccaaagctcaagaacaagtacctcttctggggaagatacctcacccaattccagatgttccagttcatgctcaacttggtgcaagcttactacgatatgaaaaccaacgctccatatccacaatggctcatcaagatcctcttctactacatgatctccctcttgttcctcttcggaaacttctacgtgcaaaagtacatcaagccatccgatggaaagcaaaagggagctaagaccgagtga

SEQ ID NO: 4 - protein, 290 aa, Physcomitrella patens
MEVVERFYGELDGKVSQGVNALLGSFGVELTDTPTTKGLPLVDSPTPIVLGVSVYLTIVIGGLLWIKARDLKPRASEPFLLQALVLVHNLFCFALSLYMCVGIAYQAITWRYSLWGNAYNPKHKEMAILVYLFYMSKYVEFMDTVIMILKRSTRQISFLHVYHHSSISLIWWAIAHHAPGGEAYWSAALNSGVHVLMYAYYFLAACLRSSPKLKNKYLFWGRYLTQFQMFQFMLNLVQAYYDMKTNAPYPQWLIKILFYYMISLLFLFGNFYVQKYIKPSDGKQKGAKTE

SEQ ID NO: 5 - DNA, 1320 bp, Thraustochytrium sp.
atgggaaaaggatctgagggaagatctgctgctagagagatgactgctgaggctaacggagataagagaaagaccatcctcattgagggagtgttgtacgatgctaccaacttcaaacacccaggaggttccattattaacttcctcaccgagggagaagctggagttgatgctacccaagcttacagagagttccatcagagatccggaaaggctgataagtacctcaagtccctcccaaagttggatgcttctaaggtggagtctaggttctctgctaaggagcaggctagaagggacgctatgaccagggattacgctgctttcagagaggagttggttgctgagggatacttcgatccatctatcccacacatgatctacagagtggtggagattgtggctttgttcgctttgtctttctggttgatgtctaaggcttctccaacctctttggttttgggagtggtgatgaacggaatcgctcaaggaagatgcggatgggttatgcatgagatgggacacggatctttcactggagttatctggctcgatgataggatgtgcgagttcttctacggagttggatgtggaatgtctggacactactggaagaaccagcattctaagcaccatgctgctccaaacagattggagcacgatgtggatttgaacaccttgccactcgttgctttcaacgagagagttgtgaggaaggttaagccaggatctttgttggctttgtggctcagagttcaggcttatttgttcgctccagtgtcttgcttgttgatcggattgggatggaccttgtacttgcacccaagatatatgctcaggaccaagagacatatggagtttgtgtggatcttcgctagatatatcggatggttctccttgatgggagctttgggatattctcctggaacttctgtgggaatgtacctctgctctttcggacttggatgcatctacatcttcctccaattcgctgtgtctcatacccatttgccagttaccaacccagaggatcaattgcattggcttgagtacgctgctgatcataccgtgaacatctctaccaagtcttggttggttacctggtggatgtctaacctcaacttccaaatcgagcatcatttgttcccaaccgctccacaattcaggttcaaggagatctctccaagagttgaggctctcttcaagagacataacctcccttactacgatttgccatacacctctgctgtttctactaccttcgctaacctctactctgttggacattctgttggagctgataccaagaagcaggattga

SEQ ID NO: 6 - protein, 439 aa, Thraustochytrium sp.
MGKGSEGRSAAREMTAEANGDKRKTILIEGVLYDATNFKHPGGSIINFLTEGEAGVDATQAYREFHQRSGKADKYLKSLPKLDASKVESRFSAKEQARRDAMTRDYAAFREELVAEGYFDPSIPHMIYRVVEIVALFALSFWLMSKASPTSLVLGVVMNGIAQGRCGWVMHEMGHGSFTGVIWLDDRMCEFFYGVGCGMSGHYWKNQHSKHHAAPNRLEHDVDLNTLPLVAFNERVVRKVKPGSLLALWLRVQAYLFAPVSCLLIGLGWTLYLHPRYMLRTKRHMEFVWIFARYIGWFSLMGALGYSPGTSVGMYLCSFGLGCIYIFLQFAVSHTHLPVTNPEDQLHWLEYAADHTVNISTKSWLVTWWMSNLNFQIEHHLFPTAPQFRFKEISPRVEALFKRHNLPYYDLPYTSAVSTTFANLYSVGHSVGADTKKQD

SEQ ID NO: 7 - DNA, 903 bp, Ostreococcus tauri
atgtctgcttctggagctttgttgcctgctattgctttcgctgcttacgcttacgctacctacgcttatgctttcgagtggtctcatgctaacggaatcgataacgtggatgctagagagtggattggagctttgtctttgagactccctgcaattgctaccaccatgtacctcttgttctgccttgtgggacctagattgatggctaagagggaggcttttgatcctaagggattcatgctcgcttacaacgcttaccaaaccgctttcaacgttgtggtgctcggaatgttcgctagagagatctctggattgggacaacctgtttggggatctactatgccttggagcgataggaagtccttcaagattttgttgggagtgtggctccattacaacaataagtacctcgagttgttggatactgtgttcatggtggctaggaaaaagaccaagcagctctctttcttgcatgtgtaccatcatgctttgttgatttgggcttggtggcttgtttgtcatctcatggctaccaacgattgcatcgatgcttatttcggagctgcttgcaactctttcatccacatcgtgatgtactcctactacctcatgtctgctttgggaattagatgcccttggaagagatatatcacccaggctcagatgttgcaattcgtgatcgtgttcgctcatgctgttttcgtgctcagacaaaagcactgccctgttactttgccttgggcacaaatgttcgtgatgacaaatatgttggtgctcttcggaaacttctacctcaaggcttactctaacaagtctaggggagatggagcttcttctgttaagcctgctgagactactagagcaccttctgtgagaagaaccaggtccaggaagatcgattga

SEQ ID NO: 8 - protein, 300 aa, Ostreococcus tauri
MSASGALLPAIAFAAYAYATYAYAFEWSHANGIDNVDAREWIGALSLRLPAIATTMYLLFCLVGPRLMAKREAFDPKGFMLAYNAYQTAFNVVVLGMFAREISGLGQPVWGSTMPWSDRKSFKILLGVWLHYNNKYLELLDTVFMVARKKTKQLSFLHVYHHALLIWAWWLVCHLMATNDCIDAYFGAACNSFIHIVMYSYYLMSALGIRCPWKRYITQAQMLQFVIVFAHAVFVLRQKHCPVTLPWAQMFVMTNMLVLFGNFYLKAYSNKSRGDGASSVKPAETTRAPSVRRTRSRKID

SEQ ID NO: 9 - DNA, 1197 bp, Phytophthora sojae
atggctattttgaaccctgaggctgattctgctgctaacctcgctactgattctgaggctaagcaaagacaattggctgaggctggatacactcatgttgagggtgctcctgctcctttgcctttggagttgcctcatttctctctcagagatctcagagctgctattcctaagcactgcttcgagagatctttcgtgacctccacctactacatgatcaagaacgtgttgacttgcgctgctttgttctacgctgctaccttcattgatagagctggagctgctgcttatgttttgtggcctgtgtactggttcttccagggatcttacttgactggagtgtgggttatcgctcatgagtgtggacatcaggcttattgctcttctgaggtggtgaacaacttgattggactcgtgttgcattctgctttgttggtgccttaccactcttggagaatctctcacagaaagcaccattccaacactggatcttgcgagaacgatgaggttttcgttcctgtgaccagatctgtgttggcttcttcttggaacgagaccttggaggattctcctctctaccaactctaccgtatcgtgtacatgttggttgttggatggatgcctggatacctcttcttcaacgctactggacctactaagtactggggaaagtctaggtctcacttcaacccttactccgctatctatgctgatagggagagatggatgatcgtgctctccgatattttcttggtggctatgttggctgttttggctgctttggtgcacactttctccttcaacaccatggtgaagttctacgtggtgccttacttcattgtgaacgcttacttggtgttgattacctacctccaacacaccgatacctacatccctcatttcagagagggagagtggaattggttgagaggagctttgtgcactgtggatagatcatttggtccattcctcgattctgtggtgcatagaatcgtggatacccatgtttgccaccacatcttctccaagatgcctttctatcattgcgaggaggctaccaacgctattaagcctctcctcggaaagttctacttgaaggataccactcctgttcctgttgctctctggagatcttacacccattgcaagttcgttgaggatgatggaaaggtggtgttctacaagaacaagctctag

SEQ ID NO: 10 - protein, 398 aa, Phytophthora sojae
MAILNPEADSAANLATDSEAKQRQLAEAGYTHVEGAPAPLPLELPHFSLRDLRAAIPKHCFERSFVTSTYYMIKNVLTCAALFYAATFIDRAGAAAYVLWPVYWFFQGSYLTGVWVIAHECGHQAYCSSEVVNNLIGLVLHSALLVPYHSWRISHRKHHSNTGSCENDEVFVPVTRSVLASSWNETLEDSPLYQLYRIVYMLVVGWMPGYLFFNATGPTKYWGKSRSHFNPYSAIYADRERWMIVLSDIFLVAMLAVLAALVHTFSFNTMVKFYVVPYFIVNAYLVLITYLQHTDTYIPHFREGEWNWLRGALCTVDRSFGPFLDSVVHRIVDTHVCHHIFSKMPFYHCEEATNAIKPLLGKFYLKDTTPVPVALWRSYTHCKFVEDDGKVVFYKNKL

SEQ ID NO: 11 - DNA, 1086 bp, Phytophthora infestans
atggctacaaaggaggcttacgttttcccaactctcaccgagatcaagagatctctcccaaaggattgcttcgaggcttctgtgcctttgtctctctactacactgtgagatgcttggttattgctgtggctttgaccttcggattgaactacgctagagctttgccagaggttgagtctttctgggctttggatgctgctttgtgcactggatatatcctcctccagggaattgtgttctggggattcttcactgttggacacgatgctggacacggagctttctctagataccacctcttgaacttcgttgtgggaaccttcatgcactctctcatcttgaccccattcgagtcttggaagttgacccacagacaccaccacaagaacaccggaaacatcgatagagatgaggtgttctacccacagagaaaggctgatgatcacccattgtccaggaacttgatcttggctttgggagctgcttggcttgcttatttggtggagggattcccaccaagaaaggtgaaccacttcaacccattcgagccactttttgtgagacaagtgtccgctgtggttatctctttgctcgctcacttcttcgttgctggactctctatctacttgtctctccagttgggacttaagaccatggctatctactactacggaccagttttcgtgttcggatctatgttggtgattaccaccttcttgcaccacaacgatgaggagactccatggtatgctgattctgagtggacttacgtgaagggaaacttgtcctctgtggatagatcttacggtgctctcatcgataacctctcccacaacatcggaactcaccagatccaccacctcttcccaattatcccacactacaagctcaagaaggctactgctgctttccaccaagctttcccagagcttgtgagaaagtccgatgagccaatcatcaaggctttcttcagagtgggaaggttgtatgctaactacggagtggttgatcaagaggctaagctcttcactttgaaggaggctaaggctgctactgaagctgctgctaagaccaagtctacctga

SEQ ID NO: 12 - protein, 361 aa, Phytophthora infestans
MATKEAYVFPTLTEIKRSLPKDCFEASVPLSLYYTVRCLVIAVALTFGLNYARALPEVESFWALDAALCTGYILLQGIVFWGFFTVGHDAGHGAFSRYHLLNFVVGTFMHSLILTPFESWKLTHRHHHKNTGNIDRDEVFYPQRKADDHPLSRNLILALGAAWLAYLVEGFPPRKVNHFNPFEPLFVRQVSAVVISLLAHFFVAGLSIYLSLQLGLKTMAIYYYGPVFVFGSMLVITTFLHHNDEETPWYADSEWTYVKGNLSSVDRSYGALIDNLSHNIGTHQIHHLFPIIPHYKLKKATAAFHQAFPELVRKSDEPIIKAFFRVGRLYANYGVVDQEAKLFTLKEAKAATEAAAKTKST

SEQ ID NO: 13 - DNA, 1086 bp, Hyaloperonospora parasitica
atggctactaaacaatcagttgcttttcctactttgactgatcttaaaagatctcttccttctgagtgttttgaatcttctttgcctctttctctttactatacacttagatctttggtttttgctggttctcttgctgtttctctttcttacgctcttgctcaacctttggttcaaaacttttaccctcttagagttgctcttattgctggatacactgtttttcaaggagttattttctggggatttttcactattggtcatgatgctggtcatggtgctttttctagatatcctgttcttaacttcactgttggaacacttatgcattctcttattttgactccttttgaatcttggaagttgactcatagacatcatcataaaaacactggaaatatcgatagagatgagatcttctaccctcaaagagaatctgatgatcatcctgtttctagacatcttactttcactcttggagctgcttggttcgcttaccttgttgagggttttccacctagaaaattgaatcattacaatcctttcgagccattgttcgagagaagagtttctgctgttgttatctctatcttggctcagtttttcgttgcaggattgtctatttacttgtgtttccaggttggagttcaggctgttgctctttactattacggtcctatcttcgtttttggtactatgcttgttattactacttttcttcatcataacgatgaagagactccttggtacggtgatgaggattggtcttacgttaagggtaacttgtcttctgttgatagatcttacggtcctcttatcgataacttgtctcataacatcggtactcatcaagttcatcatcttttcccaatcatccctcattacaaattaaagcctgctacagctgctttcagaagagctttcccacatcttgttagaaagtctgatgaaagaattttgcaggctttttacagaattggtagattgtatgctaaatatggtgttgctgattcttctgctaaattgtttacattgaaggaagctcaacttacttctaaagctgcttctgatgctaaagctgcttga

SEQ ID NO: 14 - protein, 361 aa, Hyaloperonospora parasitica
MATKQSVAFPTLTDLKRSLPSECFESSLPLSLYYTLRSLVFAGSLAVSLSYALAQPLVQNFYPLRVALIAGYTVFQGVIFWGFFTIGHDAGHGAFSRYPVLNFTVGTLMHSLILTPFESWKLTHRHHHKNTGNIDRDEIFYPQRESDDHPVSRHLTFTLGAAWFAYLVEGFPPRKLNHYNPFEPLFERRVSAVVISILAQFFVAGLSIYLCFQVGVQAVALYYYGPIFVFGTMLVITTFLHHNDEETPWYGDEDWSYVKGNLSSVDRSYGPLIDNLSHNIGTHQVHHLFPIIPHYKLKPATAAFRRAFPHLVRKSDERILQAFYRIGRLYAKYGVADSSAKLFTLKEAQLTSKAASDAKAA

SEQ ID NO: 15 - DNA, 1653 bp, Thalassiosira pseudonana
atgggtaatggtaatcttccagcatctacagcacaactcaagtcaacaagtaaacctcaacagcaacacgagcacagaacaatcagtaaatctgaattggcacaacataacactcctaagtctgcttggtgtgcagttcattcaactcctgctacagatccaagtcactctaataacaaacagcatgcacaccttgttttggatattacagatttcgcttctagacatccaggaggagatttgattcttttggcttcaggaaaagatgcaagtgtgctcttcgagacctaccaccctaggggagttccaacttcattaattcaaaagcttcagatcggtgttatggaagaggaagcttttagagatagtttctactcttggacagattctgatttctacaccgttcttaagagaagggttgtggaaagattagaggaaaggggacttgatagaaggggttcaaaagagatttggatcaaggctttatttctcttagttggattctggtactgtctttacaagatgtacactacatcagatatagatcaatacggaatagctattgcatatagtatcggaatgggtacttttgctgcattcatcggtacatgcatacaacatgatggaaaccacggtgctttcgcacagaacaagcttttgaacaagttggctggatggacactcgatatgatcggtgcttctgcattcacctgggaattgcagcatatgctcggtcatcacccttacactaatgttcttgatggagtggaggaagagagaaaagaaaggggagaggatgtggctttggaagagaaggatcaagagtcagatccagatgttttctcttcattccctctcatgagaatgcatccacatcacaccactagttggtaccataaatatcagcacctttatgctcctccactctttgcattaatgacccttgctaaggtgtttcaacaggatttcgaagttgcaacatctggaagattgtaccatattgatgctaacgttagatatggttcagtttggaatgtgatgagattctgggctatgaaagttatcacaatgggatacatgatgggtttgcctatttactttcatggagttctcaggggagtgggtcttttcgttatcggacaccttgcatgtggtgaactcttagctactatgttcatagttaaccatgtgattgagggagtgagttatggtacaaaagatcttgttggaggtgcatctcacggagatgaaaagaaaattgtgaagcctacaaccgttttaggtgataccccaatggagaaaactagagaagaggctctcaagtcaaacagtaacaacaacaagaaaaagggagaaaagaactcagttcctagtgtgccatttaatgattgggctgcagtgcaatgccagacttctgttaactggtctcctggttcatggttttggaatcatttcagtggaggtttgtctcaccaaatcgagcatcacctcttcccaagtatatgtcatactaactactgccacattcaagatgttgtggaatctacatgtgctgagtacggtgtgccatatcagtctgaatcaaacttgttcgttgcatacggaaagatgatctcacatttgaagttcctcggtaaggctaagtgcgagtga

SEQ ID NO: 16 - protein, 360 aa, Thalassiosira pseudonana
MGNGNLPASTAQLKSTSKPQQQHEHRTISKSELAQHNTPKSAWCAVHSTPATDPSHSNNKQHAHLVLDITDFASRHPGGDLILLASGKDASVLFETYHPRGVPTSLIQKLQIGVMEEEAFRDSFYSWTDSDFYTVLKRRVVERLEERGLDRRGSKEIWIKALFLLVGFWYCLYKMYTTSDIDQYGIAIAYSIGMGTFAAFIGTCIQHDGNHGAFAQNKLLNKLAGWTLDMIGASAFTWELQHMLGHHPYTNVLDGVEEERKERGEDVALEEKDQESDPDVFSSFPLMRMHPHHTTSWYHKYQHLYAPPLFALMTLAKVFQQDFEVATSGRLYHIDANVRYGSVWNVMRFWAMKVITMGYM

SEQ ID NO: 17 - DNA, 1377 bp, Ostreococcus RCC809
atgccgacgactcgatcgcgcgcgcgcgtgacgacgccccctcgcgagacgccgacgagagcgaacaccgtcgccgcgctcgatcccgagcgcaagtacacgcgcattcgcggcgtcgtgtacgacgtcacggatttcgccagccgtcatccgggtggcgcgcaattgttatcgctgtgcgtggggagagacgccaccatcctggtggagagtcatcaccttcgtccggaggtggtgcaaaagtacctgaagacgcttcccgtggtggagggcgcggcgggggcgttcgggcccgaggagacgtttccgaaaccgctcgactcggatttgtaccgaaagattcaggggcgcgttcgtaaagagatcgtcgaaccgttgaagatgacgcgcggacgcgagccgcacgggcgaggctggtgcgtgttggacgccggggtggtgttggctttcttcgcgttcgcgttgggagtctattggaagacgccgacggtggcgacggggtgcctgttggggctcgccgggtactggagcggcaccggattgcaacacacggcgaaccacggtggattggcgaagagtgggttttggaatcagttttggggatggctcgggaacgacgtcgccatcgggaagagctcggtggagtggagatatcatcacatggtgagccaccactcgtattgcaacgacgcggacctcgatcaagacgtgtacaccgcgctgccgcttcttcgtttggacccgtcccaggagttgaagtggttccaccgctaccaagcgttctacgcgccgctgatgtggccgatgttgtggctcgccgcgcagtttggcgacgcgcaaaatattttagtggataaggcgtctccgggcgtcgagtacaagggcctcatgaagctcgaagtcgcgctgtacgttctcggaaagtttttgcattttagcttgttgctcggcgtaccggcctacttgcacgggtttgcgaacgccatcgtgccgttcatcgcgtacggtgcgttcggttcgttcgtcctgtgctggtttttcatcgtcagtcacaacttggaggcgttgaccccaatcaatctgagcaaatccacgaagaatgactggggcgcgtggcaaatcgaaacttccgcgtcctggggcaacggcttctggagctttttctccggcgggttgaatttgcaaatcgagcaccacttgttcccgggttgcgcgcacaacttgtacccgaagatggttcccatcatcaaggaagagtgcgaaaaggctggcgtcacgtacaccggttacggtgggtactttggtctccttcccatcactcgggacatgttcgcgtacttgtacaaaatgggccgacaaagcaaaaagtcggcgtaa

SEQ ID NO: 18 - protein, 458 aa, Ostreococcus RCC809
MPTTRSRARVTTPPRETPTRANTVAALDPERKYTRIRGVVYDVTDFASRHPGGAQLLSLCVGRDATILVESHHLRPEVVQKYLKTLPVVEGAAGAFGPEETFPKPLDSDLYRKIQGRVRKEIVEPLKMTRGREPHGRGWCVLDAGVVLAFFAFALGVYWKTPTVATGCLLGLAGYWSGTGLQHTANHGGLAKSGFWNQFWGWLGNDVAIGKSSVEWRYHHMVSHHSYCNDADLDQDVYTALPLLRLDPSQELKWFHRYQAFYAPLMWPMLWLAAQFGDAQNILVDKASPGVEYKGLMKLEVALYVLGKFLHFSLLLGVPAYLHGFANAIVPFIAYGAFGSFVLCWFFIVSHNLEALTPINLSKSTKNDWGAWQIETSASWGNGFWSFFSGGLNLQIEHHLFPGCAHNLYPKMVPIIKEECEKAGVTYTGYGGYFGLLPITRDMFAYLYKMGRQSKKSA

SEQ ID NO: 19 - DNA, 1186 bp, Perilla frutescens
ccatggctgtttcttctggagctaggctttctaagtctggagctgatggagaagttttcgatggacagcaacagtacgagggaattggaaagagagctgctgataagtttgatcctgctgctcctcctcctttcaagatcgctgatatcagggctgctattcctgctcattgctgggttaagaacccttggaggagtctttcttacgttgtgtgggatgttgctgctgttttcgctcttcttgctgctgctgtgtacattaactcttgggctttctggcctgtttactggattgctcagggaactatgttctgggctcttttcgttcttggacacgattgtggacacggatctttctctgataacactactcttaacaacgttgtgggacacgttcttcactcttctatccttgtgccttaccacggatggagaatctctcataggactcaccatcagaaccatggacacgttgagaaggatgagtcttgggttccacttcctgagaacctttacaagaagcttgatttctctactaagttccttaggtacaagatccctttccctatgttcgcttaccctctttacctttggtacagatctcctggaaagactggatctcacttcaacccttactctgatcttttcaagcctaacgagaggggacttatcgtgacttctactatgtgttgggctgctatgggagtgtttcttctttacgcttctactatcgtgggtcctaacatgatgttcaagctttacggagtgccttaccttattttcgtgatgtggcttgatactgtgacttaccttcaccaccacggatacgataagaagcttccttggtacaggtcaaaggagtggtcttaccttagaggaggacttactactgtggatcaggattacggattcttcaacaagatccaccacgatattggaactcacgtgatccatcaccttttccctcagattcctcactaccaccttgttgaggctactagagaggctaagagggtgttgggaaactactaccgtgagcctagaaagtctggacctgtgcctcttcatcttatccctgctcttttgaagtctcttggaagggatcactacgtgtctgataacggagatatcgtgtactaccagactgatgatgagcttttcccttctaagaagatctgattaattaa

SEQ ID NO: 20 - protein, 391 aa, Perilla frutescens
MAVSSGARLSKSGADGEVFDGQQQYEGIGKRAADKFDPAAPPPFKIADIRAAIPAHCWVKNPWRSLSYVVWDVAAVFALLAAAVYINSWAFWPVYWIAQGTMFWALFVLGHDCGHGSFSDNTTLNNVVGHVLHSSILVPYHGWRISHRTHHQNHGHVEKDESWVPLPENLYKKLDFSTKFLRYKIPFPMFAYPLYLWYRSPGKTGSHFNPYSDLFKPNERGLIVTSTMCWAAMGVFLLYASTIVGPNMMFKLYGVPYLIFVMWLDTVTYLHHHGYDKKLPWYRSKEWSYLRGGLTTVDQDYGFFNKIHHDIGTHVIHHLFPQIPHYHLVEATREAKRVLGNYYREPRKSGPVPLHLIPALLKSLGRDHYVSDNGDIVYYQTDDELFPSKKI

SEQ ID NO: 21 - DNA, 1360 bp, Mantoniella squamata
ccatgggacctcctaaggaatctactcgtaagaacgctggcggacctctcacccgtggcaagctctctgctgacctcgctaagctcgaacctcacaagctcgctcagacattcgacactcgttgggtgagggttggagacgttgagtacgacgtcaccaacttcaagcatcctggtggatccgtgatcttctacatgctcagtaacactggtgctgacgccaccgaggctttcaacgagttccacatgcgttcccctaaggcttggaagatgctcaaggccctccctaaccgtcctgctgagacacctaggagtcaggaccctgacggtcctatgctcgaagacttcgctaagtggcgtgcccagctcgaaaaggaaggattcttcaagccttctatcgctcacgtcgcctacaggatcgccgaactcgctgccatgttcgctctcggctgctacatcatgtctctcggttaccctgtcgtggcttccatcgtgttcggcgctttcttcggtgcccgttgcggatgggtccagcacgaaggcggtcataactctctcaccggaaacatctggctcgacaagcgtatccaggctgccacctgcggtttcggactctctacatccggcgacatgtggaaccagatgcacaacaagcaccatgctacacctcagaaggtgcgtcatgacatggacctcgacaccacacctgctgttgccttcttcaagactgccgtcgaggacaaccgtcctaggggcttctcccgtgcttggagtagggctcaggcctggacattcgtccctgtgacttctggtctcctcgtccagatgttctggatctacgtcctccaccctcgtcaggtggccaggaagaagaactacgaggaggcttcttggatgatcctctcccatgtgctccgtactgctaccatcaagtacgccggaggctacagttggcctgtcgcctacctctggttctctttcggtaactggatcgcctacatgtacctcttcgctcacttcagtacctctcacacacacctcgaagttgtccctagtgacaagcacatctcttgggtgaactacgctgttgaccataccgtcgacatcgacccttctaagggatacgtgaactggctcatgggctacctcaactgccaggttatccaccatctcttccctgacatgcctcagttccgtcagcctgaagtgtcccgtaggttcgttgctttcgccaagaagtggaacctcaactacaaggtcctcacttactacggtgcttggaaggccaccttcacaaacctcgacacagttggtcagcactactacaagcacggaaaggcccacgcccactaattaattaa

SEQ ID NO: 22 - protein, 449 aa, Mantoniella squamata
MGPPKESTRKNAGGPLTRGKLSADLAKLEPHKLAQTFDTRWVRVGDVEYDVTNFKHPGGSVIFYMLSNTGADATEAFNEFHMRSPKAWKMLKALPNRPAETPRSQDPDGPMLEDFAKWRAQLEKEGFFKPSIAHVAYRIAELAAMFALGCYIMSLGYPVVASIVFGAFFGARCGWVQHEGGHNSLTGNIWLDKRIQAATCGFGLSTSGDMWNQMHNKHHATPQKVRHDMDLDTTPAVAFFKTAVEDNRPRGFSRAWSRAQAWTFVPVTSGLLVQMFWIYVLHPRQVARKKNYEEASWMILSHVLRTATIKYAGGYSWPVAYLWFSFGNWIAYMYLFAHFSTSHTHLEVVPSDKHISWVNYAVDHTVDIDPSKGYVNWLMGYLNCQVIHHLFPDMPQFRQPEVSRRFVAFAKKWNLNYKVLTYYGAWKATFTNLDTVGQHYYKHGKAHAH

SEQ ID NO: 23 - DNA, 1320 bp, Ostreococcus RCC809
atgggaaagggagcaaggaacccaggagcaagggcatggaagtcaacattggagcctcacgcagtggcaaagtcattcgataggagatgggttaaggtggatggagttgaatacgatgtgactgatttcaagcatcctggaggtagtgttatatactacatgctttctaacacaggtgctgatgcaaccgaagcttttaaggagttccattacaggagtaagaaagctaggaaagcacttgctgcattgcctcaaagagaaccagaggatgcttcaccagttgaagatgcaaacatgctcaaggatttcgctaagtggagaaaggatctcgaaagggagggatttttcaaaccttctccagctcatgtggcatatagatttgctgagcttgctgcaatgttcgctctcggtacagcattaatgtacgctagatggcacgcaacttctgttttcgtgacagcttgtttctttggagcaagatgcggttgggttcaacatgagggaggtcactcttcattgactggatcaatctggtgggataagagaatacaggcttttacagcaggattcggtctcgctagttctggtgatatgtggaatttaatgcataacaagcatcacgcaacccctcaaaaagttaggcacgatatggatttggatactacaccagctgttgcatttttcaatactgctgtggaagagaacagacctaggaagttttctaaactttggttgagagttcaggcttggaccttcgttcctgtgacttcaggactcgtgcttttggcttggatgtatctcttacatccaagacacattgcaagaaggaagaattacgaagaggctgcatggatcgttgctgcacatgtgataaggacatcagttattaaagctgtgacaggatatagttggataacctgttacggtctctttttaagtaccatgtgggtttctggatgctatctttttgctcatttctcaaccagtcatactcaccttgatgttgtgccttcagataagcatttgagttgggttagatatgctgtggatcacactattgatatcgatccatctaaatcagttgtgaattggcttatgggttacttgaactgtcaggttatccatcacttgtttcctgatatgccacaattcagacagccagaagtttctagaaggtttgtgtcattcgctaagaaatggaatctcaactacaaggttatgtcttattacggagcttggaaagcaacattcggtaaccttaacgaagttggaaagcactactatattcagggttctcaaatcacaaaaaagaccgtgtaa

SEQ ID NO: 24 - protein, 439 aa, Ostreococcus RCC809
MGKGARNPGARAWKSTLEPHAVAKSFDRRWVKVDGVEYDVTDFKHPGGSVIYYMLSNTGADATEAFKEFHYRSKKARKALAALPQREPEDASPVEDANMLKDFAKWRKDLEREGFFKPSPAHVAYRFAELAAMFALGTALMYARWHATSVFVTACFFGARCGWVQHEGGHSSLTGSIWWDKRIQAFTAGFGLASSGDMWNLMHNKHHATPQKVRHDMDLDTTPAVAFFNTAVEENRPRKFSKLWLRVQAWTFVPVTSGLVLLAWMYLLHPRHIARRKNYEEAAWIVAAHVIRTSVIKAVTGYSWITCYGLFLSTMWVSGCYLFAHFSTSHTHLDVVPSDKHLSWVRYAVDHTIDIDPSKSVVNWLMGYLNCQVIHHLFPDMPQFRQPEVSRRFVSFAKKWNLNYKVMSYYGAWKATFGNLNEVGKHYYIQGSQITKKTV

SEQ ID NO: 25 - DNA, 1430 bp, Ostreococcus RCC809
ggatccggtaccaagcttgatatcaccaaaatgccaactactcgttctcgtgctcgtgttactactccacctcgtgaaactcctactcgtgctaatactgttgctgctttagatccagaacgtaaatatacacgtattcgaggtgttgtatatgatgttactgattttgctagtcgacatccaggtggtgcacaattattatctttatgtgttggtcgtgatgctacaattttagtagaatcacatcatttacgaccagaagttgtacaaaaatatttaaaaacattacctgttgtagaaggtgctgctggtgcatttggtccagaagaaacttttccaaaacctttagatagtgatttatatcgtaaaattcaaggtcgtgttcgaaaagaaattgtagaaccattaaaaatgacacgtggtcgagaacctcatggtcgtggttggtgtgttttagatgctggtgttgtattagctttctttgcttttgcattaggtgtttattggaaaacaccaactgtagctactggttgtttattaggtttagcaggttattggtctggtacaggtttacaacatactgctaatcatggtggtttagcaaaatcaggttttggaatcaattttggggttggttaggaaatgatgttgctattggtaaatcaagtgtagaatggcgttatcatcatatggtttcacatcatagttattgtaatgatgctgatttagatcaagatgtttatacagcattaccattattacgtttagatccttcacaagaattaaaatggtttcatcgttatcaagcattttatgcacctttaatgtggcctatgttatggttagctgcacaatttggtgatgctcaaaatattttagttgataaagcaagtccaggtgtagaatataaaggtttaatgaaattagaagttgctttatatgtattaggaaaatttttacatttttctttattattaggtgttcctgcatatttacatggttttgctaatgcaattgtaccatttattgcttatggtgcatttggttcatttgttttatgttggtttttcattgtaagtcataatttagaagcattaacaccaattaatttatctaaatcaactaaaaatgattggggtgcttggcaaattgaaactagtgcatcttggggtaatggtttttggtcatttttctcaggtggtttaaatttacaaattgaacatcatttatttcctggttgtgctcataatttatatccaaaaatggttcctattattaaagaagaatgtgaaaaagcaggtgttacatatactggttatggtggttattttggtttattaccaattactcgtgatatgtttgcttatttatataaaatgggtcgtcaatctaaaaaatctgcttaagagctcggtaccctcgagtctaga

SEQ ID NO: 26 - DNA, 23 bp, Artificial Sequence
  sgRNA g-RNA-F inserted into pHEE401E vector
attgttggagatgggaatagaag

SEQ ID NO: 27 - DNA, 23 bp, Artificial Sequence
  sgRNA g-RNA-R inserted into pHEE401E vector
aaaccttctattcccatctccaa

SEQ ID NO: 28 - DNA, 6474 bp, Camelina sativa
  misc_feature (2508)..(2508): n is a, c, g, or t
atttcgaaatatttggccttaactaaacttccaccatgatttatttactgatctagttcggggacagactttgcgaataaaactcattaccgagaaacattcatcccataattgctatttagtcagaggctaatcgactatggcctttcagccaatcaaagctacgaacacgaatctccctaaaacatcctcaagtattttatttaatacacatgtatcgtattgagcaccactcataaactaatttcatacatttatcatactctttatttgtaataataaaagcatcaacatattgtaggcaattagaatcaaaacaaaacattttttttttctttccaaattttcaaaattggtaaacgaaacttggacctttaatacttatattggcaatataataatattgcagagtggactatttcccttattttggcaactttcagtggactagtaatttatttcaatgtggatgcttgcatgagtgtgaatatacacatgtctatatgcatgcctgcaaatcgtaacggaccacaaaaaaggatccatacaaatacctcttaacggctcctctctatcatactctccgacacaaactgagcaatgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggacctaattcatctatctgaatctgaatgttaaccatttagtgtttttttttttcctttccttctttcttttattggttaagatttgataatgtttatgatgttctctcgttttcgttataaataaagaaacacatgggtgtttccattcttctatttaagttgtggaccgtttaataacctaacttagaattcaatcatctttttttgttcttctgtttttaaatcttccaaccaatcaacgtgttttcatactaacataattattaaaacgtgtttaaaattaaaaatctctttggtgtttctagaggttttagattgtttgctctgtttgccacctgtttttttttccccccgtcagctcctgtatttgagatttagatatataaatgcatatnaaactaaaaaaaactagaaaaagaaataaagaaagcaatatacctctgcttgtgatatggaagtggaagaaatacaagatttgatcgattctcatcatcttgagatgactgaatagtcggaaccatggaaacaccaaacacacaggatcatgactggttctctactctcactcgtcctcgttcctgcattctcgcatccttgagccgtcgttgatatatccaccgtgtgaaaacgtttcggtgatcgtagagaaaattttgacctaatttaaatcgatgacgatcggatgaagatgatgtagtcacgatttaacaagagagattaatcacgaaaagagatgctctccgctccgatcaaatatcaaaaagagatcatctctttttgtgattgtgtaagaagttagagagaagacttgacgtcctgtaatcgtgatctcgatcgaatgaagatatgtcgtcacgattaacatgaggatggcgatcaatcaatcacgaacagagtcagagatgcttcccctaatcttgacagagatcaccaacgcaaagctttggatttggtttctcaaagaagaagaagaagagagatttggaaaactttgacaaagaagaaaatgagagaatgaatgaatgatgtgatgttggagattttttaacctaatgactaaatgagccgtcttttatatacgcaacagctatattaatatttttttattttttattttctaatttcagcaaatttaaataacgaaattgtgattgtttcctttgttttcttttccccacgagttctcaagttgtttgttttcttattttggttgcaaccaaaaaaaacaagacagaaaaaacaatttatctcaagagtaacaaaaaggagattcgagatcctttggagttactcaattacattcatatgtcacgagataaaaaggttaaacaatcactggatcagtgtgctcatggtgttccaggtccaaaacatgtgtcacgagataaaaacaaagagaaacacaactgaaaatgattaccaagatcacaactatatataaaggacttataaaaaatgaatttgaaagtggttaaactaagtgattataagtgttattgcagttacccccttataggtttggtgaatcttattagagataacttattcttaagatagttgcaattaaccaaaaaaaaaaattgtccggatagtttgatgcaattaaatgattaatgagtgttctatagggtctgattcttaatatttcgaaatatttggccttaactaaacttccaccatgatttatttactgatctagttcggggacagactttgcgaataaaactcattaccgagaaacattcatcccataattgctatttagtcagaggctaatcgactatggcctttcagccaatcaaagctacgaacacgaatctccctaaaacatcctcaagtattttatttaatacacatgtatcgtattgagcaccactcataaactaatttcatacatttatcatactctttatttgtaataataaaagcatcaacatattgtaggcaattagaatcaaaacaaaacattttttttttctttccaaattttcaaaattggtaaacgaaacttggacctttaatacttatattggcaatataataatattgcagagtggactatttcccttattttggcaactttcagtggactagtaatttatttcaatgtggatgcttgcatgagtgtgaatatacacatgtctatatgcatgcctgcaaatcgtaacggaccacaaaaaaggatccatacaaatacctcttaacggctcctctctatcatactctccgacacaaactgagcaatgacgtccgttaacgcaaagctcctttaccattacgttctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggacctaattcatctatctgaatctgaatgttaaccatttagtgtttttttttttcctttccttctttcttttattggttaagatttgataatgtttatgatgttctctcgttttcgttataaataaagaaacacatgggtgtttccattcttctatttaagttgtggaccgtttaataacctaacttagaattcaatcatctttttttgttcttctgtttttaaatcttccaaccaatcaacgtgttttcatactaacataattattaaaacgtgtttaaaattaaaaatctctttggtgtttctagaggttttagattgtttgctctgtttgccacctgtttttttttccccccgtcagctcctgtatttgagatttagatatataaatgcatatttgaattgtttaggtattaaaaaaaaaaaaaaaaaaaagcaaggaagccctaagcttccttttaaattaggtgtgtttttttactttaacttatgacgcatttttttgcaacatgtaaattattttggtattattatagctaatactaataactatgacagtaattaaattataagatatatatttgcatagaaaatagaaatatataaac

SEQ ID NO: 29 - DNA, 2916 bp, Camelina sativa
atttggccttaactaaacttccacctgatttatttactgatctagttcggggacagactttgcgaataaaactcgttcccgagaaacattcatcccataactgctatttagtcagaggctaatcgactatggcctttcagccaatcaaatctacgaacacgaatccccctaaaacatcctcaagtatttatttaatacacatgtatcgtattgagcaccactcataaactattttttttttgtttttaacaaaaaaaatttatcatactcttttgtaataatagatgcatcaacatattgtaggcaacgttgaagaaccagtacattctttttttttttgctccaaattttcaaaattggaaaatgaaacttggacgaaataaatttaacactctgtatatattggcaatataatattgcagagtggactatttaccttattttggcaactttcagtggactagtaatttatttcaatgtgtatgcttgcatgagtgtgaatatacacatgtctatatgcatgcttgcaaatcgtaacggaccacaaaaaaggatccatacaaatacctcttaacggctcctctctatcatactctccgacacaaactgagcaatgacgtccgttaacgcaaagctcctttaccattacgttctaaccaactttttcaacctttgcttgtttccgttaacggcgttacttgccggaaaagcctctaagcttacagcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacgtacagtccccagggactcattaacgtgcccccacaaaagacctttgcagcttcacgtgaagagacagagcaggtaatcatcggtgcgctagaaaagctattcgagaacaccaaagtaaaccctagagagattggtatacttgtggtgaactcaagcatgtttaatccaactccttcgctatctgcgatggtcgttaacactttcaagctccgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaacagatccatgatggttagcaattgcttgtttcgtgttggtggggcagcgattttgctctccaacaaactgggagatcggagacggtccaagtacaagctatgtcatactgttcgaacgcataccggagctgatgacaagtcttttcgatgtgtgcaacaaggagacgatgagggcggtaaaatcggagtttgtctgtcaaaggacataaccgttgttgcggggacagcgcttaagaaaaacatagcaacgttgggtccgttgattcttcctttaagcgaaaagtttctgtttttagttaccttcatcgccaagaaacttttgaaggacaagatcaagcactgttacgtcccggatttcaagcttgctatcgaccatttctgtattcatgcgggaggcagagccgtgatcgatgtgcttgagaagagcttaggactatcgccaatcgatgtggaggcatctagatcaacgttacatagatttgggaatacttcgtctagctcaatttggtatgaattggcatacatagaagcaaaaggaaggatgaagaaagggaatagagcttggcagattgctttagggtcagggtttaagtgtaacagtgcggtttgggtggctctatgcaatgtcaaggcttcggcgaatagtccttgggaacattgcatcgatagatatccggttcaaattgattctgattcatcaaaatcagagactcatgtcaaaaacggtcggacctaattcatctatctgagtgttaacaatttagtgtttttttcttctttccttctttcttttaattattggttatgatttgataatgtttaatgtgttctttctttttcgttataaataaagaaacacatgggtgtttccattcttctatttaagttgtggaccgtttaataacctaacttagaattcaatcatctttttattttccttctgtttttaaatcttccaaccaatcaacgtgttttccatactaacataattatttaaaacgtgtttaaaattaaacatctctttggtgtttctagaggttttagattgtttgctctgtttgccacctgttttttttccccgtcagctctgtttgccacctgttattgttggaaaagttacattcctgtatttgagatttagatatataaatgcatatttgaattgtttaggtatcaaataaaaaaagaagcaaggaagcccatatagttcaattgatagttgaataatgaaaaatgatatgtgtcagagagttggagcagtccccgatgagcctacatcagaaaacaaaaaaaaatcatactgctaatatattttttggctgaggctcaccttatctattaattatgacattagttacattgcacatgtaattttcaaaaaatgcgggctttagagcctgaccgtctagagaaagaagtgggtggtttagaagagcttcttctacaaatgtaactaatgtcattagttaattgtggcggcgatctattaattatgacattagttacattgcacatgtaatt

SEQ ID NO: 30 - DNA, 7046 bp, Camelina sativa
  misc_feature (2062)..(2855): n is a, c, g, or t
agcataaaattaacaaagagcaacagtcaagctacagaatcataatatatttttagtttatagggaaagccaacaatcataagtgaaaggtaagaacgttattggtatagtactaatacacagtactgtaattcttagtataaatacagatccagtgatttttactaaattgactttaggtccactggagaaactaaaaaacaattgggccctagcccattcttatttttacctccatatcctctcaattaaaataccttttcctataaatttagaaaaacagaaaccaattcaaaaaaaatctactcttctctatttctccataaaaaccaaaaaaaacttattcactcttgctcattacgtctcccattagttggccaatgcacatgatgccaaaatacattaatggcgacttgtttctgttttttagttatatcaaactccacccaaccaggagaaaagttaatggtgtttctgtccctataaacacaatacattccccttacgatatttcttcctcttcatctctctctcagtgtctatatatacgcgcacacacacccagcttcagtttcttacaagcatcagcaacaagtttccatgaactcccgaaactacgtcaagcttggttatcactacctgattactcacttcttgaagctcatgctcgtccctcctgtaatggcggttttgttcacgaacgtctccttattaactctaaaccagctccagctccagctccaatacaacctcgtcggagtcatctccctcatcacattcgccatattcggatgcacagtcttcttcatgtcacgacctagatctgtttaccttgtcgattactcctgctacttaccaccgtctgatcaaaaagttagctaccaaaaattcatgaacaactcaagtttgattcaagatttcagcgagtcgtcgcttgagttccaacgaaagatcttggttcgatccggtctcggagaagagacttatttaccggagtctattcactgcaccccgccacgtccaactatggccgctgcgcgtgaggaagcagagcaggtaatcttcggtgcactcgataacctcttcgagaataccaaaatcaatcctagagagattggtgttcttgttgtgaactgtagtttgtttaaccctacgccttctttatcggccatgattgttaacaagtataaacttagaggaaacgtcaagagctttaacctaggtgggatgggatgtagtgctggtgttatcgccgttgatctcgctagtgacatgttacagatccttaggaacacttttgctcttgtggttagtactgagaacattactcagaattggtattttggtaacaagaaagcaatgttgatccctaattgcttgtttagagttggtggctccgcggttctgctttcgaacaagcctttggatcgaaaacggtccaagtataagcttgttcatacggtaaggactcataaaggatctgatgagaaagcattcagttgtgtgtaccaagaacaagacgagtgtttaaaaaccggagtttctttgtcgaaagatcttatggctatagccggagaagctttaaagacgaacatcaccactttgggtcctctggttcttcctataagcgagcagatcctcttctttgcgacttttgttgctaagagattgttcaatgccaagaagcagaagccttacataccagatttcaaacttgcgtttgatcatttctgtattcacgcgggaggtagagccgtgatcgatgaactagagaagagcttaaagctatcgccaatacatgtcgaggcgtctagaatgacattgcatagatttggaaacacttcctcgagctcgatatggtatgaattggcttacacggaagctaaaggaagaatgaggaaagggaacagagtttggcagattgcttttggaagcgggtttaagtgtaacagcgcggtttggatggctctccgcgatgtcgagccctcgtttaaaaatccttgggaacattgcatcgatagatatccggttaagatcgatctttgaactcgtaagaacggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacatgtcgaggcgtctagaatgacattgcatagatttggaaacacttcctcgagctcgatatggtatgaattggcttacacggaagctaaaggaagaatgaggaaagggaacagagtttggcagattgcttttggaagcgggtttaagtgtaacagcgcggtttggatggctctccgcgatgtcgagccctcgtttaaaaatccttgggaacattgcatcgatagatatccggttaagatcgatctttgaactcgtaagaacggtggattggtccggaaacatggttagtcctccatgtaccaaaaaagaaagtgaacttatatctctagttctttactaaggggtcaagaaatcagttgaaggttaatgtatgtttatatatgttgtgtataaaggtaacccttaaatgaccaagactgttgacctattaaaatttaaaatttaagttctctcgaaaaattaaatcgtttgtaaaataaaaattgtgagttagaaaaatcaaaacaatttacttacctacaagaaccttaaaattaaacttaagtattaaattttgaatacatgaaaaaatgttaaacttatgacaccaaaatttacgaactctgaggacggaaatgcttaattcagtttgtttgtttttttgttttgtaaatcaatctggtttgttttcaatgtatctaaactaaagtcgggttatgacaccaacaccaagcttatggagacaggatgttaagtcgggttagtcatcaaggtattcaaataagtaataacttttaagttttagaacttttttgtcagtaaaatattttgtataataaaaaagtttagttaaaacattaaaacagtttttactgaaaatattatttgtgaaaaaacttcttttaagtgttaaatttagaacacgtaaaaacatgttagacttatgacaccaagcttatagacccgaaaaggaaactgtgattgtttccttttccccacgagttctaagttgtttcctttacttatttgggttgcaaccaatataaaaagaaagaaaaatggaaatttatcacaaaaaggagagtcgagatctctagagttacattcatatgtgctagtgtctgaccttttgccttaatttataggcactaataaaaaaactaccaaaaaaaaaattgaagaagaacaaaaggttaaacaatcactggatcagtgtgctcatggtgttccaggtccaaaacatgtgtcacgagattaaaaaagagagaaacacaactgaaactattcaccaagatcacaactatatatataaaggacttataaaaaatcaatttgaaagtggttaaactaaacgattataagtgtgattgcacttacccccttataggtttggtgaatcttattagagataacttatttttaagatagttgcaattaaaaaaaaaaaaaaattgtccggatagtttgatgcaattaatgagtgttctatatggtctgattcttaatatttatgcaaatattatagtatttcaaagtatttggccttaactgacagactttgcaaataaaactcattcccgagaaacattcatcccataattggtatttagtcagaggctaatcgactatggcctttcagccaatcaaagctacgaacacgaatccccctaaaacatcctcaagtatttatttaatacatcgtattgagcaccactcataaactaattccatacatttatcatactgtttatttgtaataataaaagcagcaacatattgtagtttgtaggcaataagaaacaaaacaaaacatttttttttctctccaaattttcaaaattggaaaacgaaacttggaccttcaatacttatatattatatttgcaatataaaattgcagagtggactatttcccttattttggcaactttcagtggactagtaatttatttcaatgtgtatgcttgcatgagtgtgaatatacacatgtctatatgcatgcctgcaaatcgtaacggaccacaaaaaaggatccatacaaatatacctctcaacggctcctctctattatgctctccgacacaaactgagaaatgacgtccgttaacgcaaagctcctttaccattacgtcctaaccaactttttcaacctttgcttgtttccgttaacggcgttacttgccggaaaagcctctacgcttaccacaaacgatctctaccacttctattcccatctccaacacaaccttgtaaccgtaattttactctttgctttctcctctttcggtttggttctctacgttgtaacccggcgcagaccggtttacctcgttgactactcgtgctaccttccaccaccgcatctcaaagttagtgtttctaaggtcatggatattttctaccaaataagaaaagctgatacctcacgaaacgtggcatgcgatgatccatcctcgcttgatttcctgaggaagattcaagaacgttcaggtctaggtgatgaaacctacagtcccccgggactcattcacgtgcccccacaaaagacttttgcagcttcacgtgaagagacagagcaggtaatcatcggtgcgttagaaaagttattcgagaacaccaaagttaaccctagagagattggtatacttgtggtcaactcaagcatgtttaatccaactccttcgctatctgcgatggtcgttaacactttcaagctccgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgtttcgtgttggtggggcagcgattttgctatccaacaagccgggagatcggagacggtccaagtacaagctatgtcacacggttcggactcataccggagctgatgacaagtcttttcgatgtgtgcaacaaggagacgatgagagcggtaaaatcggagtttgtctgtcaaaggacataacagttgttgcggggacagcgcttaagaaaaacatagcaacgttaggtccgttgattcttcctttaagcgaaaagtttctgttcttagttaccttcatcgccaagaaacttttgaaggacaagatcaagcactattacgtcccggatttcaagcttgctattgaccatttctgtattcatgccggaggcagagccgtaatcgatgtgcttgagaagagcttaggactatcgccaatcgatgtggaggcatctagatcaacgttacatagatttgggaatacttcgtctagctcaatttggtatgaattggcatacatagaagcaaaaggaaggatgaagaaagggaatagagcttggcagattgctttagggtcagggtttaagtgtaacagtgcggtttgggtggctctatgcaatgtcaaggcttccgcgaatagtccttgggaacattgcatcgatagatatccggttcaaattgattctgattcatcaaaatcagagactcatgtcaaaaacggtcggacctaattcatctatctgagtgttaaccatttactgttttttatttttctttccttctttcttttattggttatgatttgataatgtttaatgatgttctttctctctttttcgttataaacttatataaataaagaaacacatgggtgttttcattcttctatttaagtagtggaccattttataacctaacttataactcaatcatcatttttttttttttctgttttcaactcttccaaccaatcaacgtttttttcatactaacataattattaaaatgtgtttaaaattaaacatctctttggtgtttctagaggttttagattgtttgctctgtttgccaaatgttttatttttttccccttcagctctgtttgccacctgttattgttggaaaagttacattcctgtttttgagatttagatatataaatgcatatttgaattgtttaggtatcaatattaaaaaaaaaaaaaagcaaggaagcccatatagttcaattgatagttgaataaatgaaaaatgatatgtgtcacagtgtcagagagttggagcagtcccccatgagcctacatcagaaaacaaaaaaaaaatcatactac

SEQ ID NO: 31 - protein, 975 aa, Camelina sativa
MTSVNAKLLYHYVLTNFFNLCLFPLTALLAGKASRLTSNDLYHFYSHLQHNLITVILLFAFTAFGLVLYIVTRPKPVYLVDYSCYLPPPHLKVSVSKAMDIFYQIRKADTSRNVACDDPSSLDFLRKIQERSGLGDETYSPQGLINVPPRKTFAASREETEQVIIGALDKLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKPGDRRRSKYKLCHTVRTHTGADDMSFRCVQQGDDESGKIGVCLSKDITVVAGIALKKNIATLGPLILPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSGSSKSDTHVKNASRLTSNDLYHFYSHLQHNLITVILLFAFTAFGLVLYIVTRPKPVYLVDYSCYLPPPHLKVSVSKAMDIFYQIRKADTSRNVACDDPSSLDFLRKIQERSGLGDETYSPQGLINVPPRKTFAASREETEQVIIGALDKLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKPGDRRRSKYKLCHTVRTHTGADDMSFRCVQQGDDESGKIGVCLSKDITVVAGIALKKNIATLGPLILPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSGSSKSDTHVKNGRT

SEQ ID NO: 32 - protein, 505 aa, Camelina sativa
MTSVNAKLLYHYVLTNFFNLCLFPLTALLAGKASKLTANDLYHFYSHLQHNLITVILLFAFTAFGLVLYIVTRPKPVYLVDYSCYLPPPHLKVSVSKAMDIFYQIRKADTSRNVACDDPSSLDFLRKIQERSGLGDETYSPQGLINVPPQKTFAASREETEQVIIGALEKLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKLGDRRRSKYKLCHTVRTHTGADDKSFRCVQQGDDEGGKIGVCLSKDITVVAGTALKKNIATLGPLILPLSEKFLFLVTFIAKKLLKDKIKHCYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSDSSKSETHVKNGRT

SEQ ID NO: 33 - protein, 1048 aa, Camelina sativa
MNSRNYVKLGYHYLITHFLKLMLVPPVMAVLFTNVSLLTLNQLQLQLQYNLVGVISLITFAIFGCTVFFMSRPRSVYLVDYSCYLPPSDQKVSYQKFMNNSSLIQDFSESSLEFQRKILVRSGLGEETYLPESIHCTPPRPTMAAAREEAEQVIFGALDNLFENTKINPREIGVLVVNCSLFNPTPSLSAMIVNKYKLRGNVKSFNLGGMGCSAGVIAVDLASDMLQILRNTFALVVSTENITQNWYFGNKKAMLIPNCLFRVGGSAVLLSNKPLDRKRSKYKLVHTVRTHKGSDEKAFSCVYQEQDECLKTGVSLSKDLMAIAGEALKTNITTLGPLVLPISEQILFFATFVAKRLFNAKKQKPYIPDFKLAFDHFCIHAGGRAVIDELEKSLKLSPIHVEASRMTLHRFGNTSSSSIWYELAYTEAKGRMRKGNRVWQIAFGSGFKCNSAVWMALRDVEPSFKNPWEHCIDRYPASRMTLHRFGNTSSSSIWYELAYTEAKGRMRKGNRVWQIAFGSGFKCNSAVWMALRDVEPSFKNPWEHCIDRYPLLYHYVLTNFFNLCLFPLTALLAGKASTLTTNDLYHFYSHLQHNLVTVILLFAFSSFGLVLYVVTRRRPVYLVDYSCYLPPPHLKVSVSKVMDIFYQIRKADTSRNVACDDPSSLDFLRKIQERSGLGDETYSPPGLIHVPPQKTFAASREETEQVIIGALEKLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKPGDRRRSKYKLCHTVRTHTGADDKSFRCVQQGDDESGKIGVCLSKDITVVAGTALKKNIATLGPLILPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSDSSKSETHVKNGRT

SEQ ID NO: 34 - protein, 407 aa, Adonis aestivalis
MAPVLLGLKPTLSTGSVVKETNVGSTLASPLNKTQNSRVLVLGGTGKVGGSTALALSKFSPDLRLVIGGRNREKGDAVVSKLGENSEFVEVNVDSVRSLESALEDVDLVVHAAGPFQQAEKCTVLEAAISTRTAYVDVCDNTSYSMQAKSFHDKAVAANVPAITTAGIFPGVSNVIAAELVRSARDENTEPQRLRFSYFTAGSGGAGPTSLVTSFLLLGEEVVAYSEGEKVELKPYTGKLNIDFGKGVGKRDVYLWNLPEVRSGHEILGVPTVSARFGTAPFFWNWAMVAMTTLLPPGILRDRNKIGMLANFVYPSVQIFDGIAGECLAMRVDLECANGRNTFGILSHERLSVLVGTSTAVFAMAILEGSTQPGVWFPEEPGGIAISDRELLLQRASQGAINFIMKQ

SEQ ID NO: 35 - protein, 309 aa, Adonis aestivalis
MAAAISVFSSGYSFYKNLLLDSKPNILKPPCLLFSPVVIMSPMRKKKKHGDPCICSVAGRTRNLDIPQIEEEEENVEELIEQTDSDIVHIKKTLGGKQSKRPTGSIVAPVSCLGILSMIGPAVYFKFSRLMEGGDIPVAEMGITFATFVAAAVGTEFLSAWVHKELWHESLWYIHKSHHRSRKGRFEFNDVFAIINALPAIALINYGFSNEGLLPGACFGVGLGTTVCGMAYIFLHNGLSHRRFPVWLIANVPYFHKLAAAHQIHHSGKFQGVPFGLFLGPKELEEVRGGTEELERVISRTTKRTQPST

SEQ ID NO: 36 - protein, 410 aa, Zea mays
MAIILVRAASPGLSAADSISHQGTLQCSTLLKTKRPAARRWMPCSLLGLHPWEAGRPSPAVYSSLAVNPAGEAVVSSEQKVYDVVLKQAALLKRQLRTPVLDARPQDMDMPRNGLKEAYDRCGEICEEYAKTFYLGTMLMTEERRRAIWAIYVWCRRTDELVDGPNANYITPTALDRWEKRLEDLFTGRPYDMLDAALSDTISRFPIDIQPFRDMIEGMRSDLRKTRYNNFDELYMYCYYVAGTVGLMSVPVMGIATESKATTESVYSAALALGIANQLTNILRDVGEDARRGRIYLPQDELAQAGLSDEDIFKGVVTNRWRNFMKRQIKRARMFFEEAERGVTELSQASRWPVWASLLLYRQILDEIEANDYNNFTKRAYVGKGKKLLALPVAYGKSLLLPCSLRNGQT

SEQ ID NO: 37 - DNA, 1224 bp, Adonis aestivalis
atggctccagtgcttttgggtttgaagcctactttgtcaactggttctgtggtgaaagaaacaaacgttggatcaacacttgcttcacctttgaacaaaactcagaactcaagggtgcttgtgttgggtggtacaggaaaagtgggtggatctacagcacttgctctttcaaagttctctccagaccttaggttggtgattggaggaaggaacagggagaagggtgacgcagtggtgtcaaagttgggagaaaattctgagttcgttgaggttaatgtggattctgtgaggtcacttgagtctgcacttgaggacgttgaccttgtggttcacgctgcaggtcctttccagcaagcagagaagtgcacagtgttggaggctgctatctctactaggactgcttacgtggacgtgtgcgacaacacttcttactctatgcaggctaagtcattccacgacaaggcagtggctgcaaacgtgcctgctatcacaactgcaggaatcttccctggagtgtctaacgtgatcgcagcagagttggtgaggtctgcaagggatgagaacacagagcctcaaaggttgaggttctcttacttcacagctggatctggtggagctggtccaacatcattggtgacttcattcttgttgttgggtgaggaggtggtggcttactctgagggagagaaggttgagttgaagccatatacaggaaagttgaatattgactttggaaaaggtgtgggaaagagagacgtgtacttgtggaacttgcctgaggtgaggtctggacacgagatccttggagtgcctactgtgtctgctaggttcggtacagctccattcttctggaactgggctatggtggcaatgactacattgcttcctcctggtatcttgagagacaggaataagatcggaatgttggctaattttgtttacccttctgtgcagatcttcgacggtatcgcaggagagtgccttgctatgagggttgacttggagtgcgcaaacggaagaaatactttcggtatcttgtcacacgaaaggttgtctgtgcttgtgggtacatctacagctgtgttcgctatggcaatccttgagggttctactcagcctggtgtttggttccctgaggagcctggaggtatcgcaatctctgacagggagcttcttttgcagagggcatcacagggagcaatcaacttcattatgaagcagtga

SEQ ID NO: 38 - DNA, 930 bp, Adonis aestivalis
atggcagctgctatttcagtgttctcttcaggatactcattctacaagaatttgttgttggactcaaagccaaacatccttaagcctccttgccttcttttctctcctgtggtgatcatgtctcctatgaggaagaagaagaagcacggagatccatgtatctgctctgtggcaggtaggacaaggaacttggacatcccacagatcgaagaggaggaagagaacgtggaggagttgatcgagcagactgactctgacatcgttcacatcaagaagactttgggaggtaagcagtcaaagaggccaactggatctatcgtggctcctgtgtcatgcttgggtatcttgtcaatgatcggaccagctgtgtatttcaagttctctaggcttatggagggaggagacatccctgtggctgagatgggaatcacattcgcaactttcgttgctgctgctgtgggaacagagttcttgtcagcatgggtgcacaaggagttgtggcacgagtctttgtggtacatccacaagtcacatcacaggtctaggaagggtaggtttgagttcaacgacgtgttcgctatcattaacgcccttccagctatcgcattgatcaattacggtttctcaaacgagggattgttgcctggtgcttgcttcggagtgggattgggaactacagtgtgcggtatggcttacatcttccttcacaacggattgtctcacaggagattcccagtgtggcttattgcaaacgtgccatacttccacaagttggcagctgctcaccagatccatcactcaggaaagtttcaaggagtgcctttcggattgttcttgggtccaaaggagcttgaggaggtgagaggaggaactgaggagttggagagagtgatttctaggactacaaagaggactcaaccatctacttga

SEQ ID NO: 39 - DNA, 1233 bp, Zea mays
atggctattattttggttagagctgcttcacctggactttctgctgctgattctatttctcatcaaggtactttgcaatgttcaacacttttgaaaactaaaagaccagcagctagaagatggatgccttgttctttgttgggacttcatccttgggaagctggtagaccttctccagctgtttattcttctcttgctgttaacccagctggagaagctgttgtttcttcagaacagaaggtttacgatgttgttcttaagcaggctgctttgttgaagagacaacttagaactccagttttggatgcaagacctcaagatatggatatgcctagaaatggacttaaggaggcatatgatagatgtggagaaatctgtgaagaatacgctaaaacattttatttgggtactatgcttatgactgaggagagaagaagggctatttgggctatctacgtttggtgtagaagaactgatgagcttgttgatggaccaaacgctaactacatcactcctactgctcttgatagatgggagaaaagattggaggatctttttactggaagaccttacgatatgttggatgctgctctttcagatactatctcaagattccctatcgatatccaacctttcagagatatgatcgagggtatgagatctgatcttagaaagactagatacaacaacttcgatgaattgtacatgtactgttactatgttgctggtacagttggtttgatgtctgttcctgttatgggtatcgctactgagtctaaagctactacagaatctgtttattctgctgctcttgctttgggtattgctaatcaacttacaaacatccttagagatgttggagaagatgctagaaggggaagaatctaccttccacaggatgaacttgctcaagctgggttgtcagatgaggatatcttcaagggtgttgttactaacagatggagaaacttcatgaagagacagatcaagagagctagaatgttcttcgaggaagctgaaagaggtgttactgaattgtctcaagcatcaagatggcctgtttgggcttctcttcttttgtacagacaaatccttgatgagatcgaggctaacgattataacaacttcacaaagagagcttatgttggaaagggaaagaaactcttggcccttcctgttgcttatggaaaaagccttttgcttccttgttcattgagaaatggtcagacataa